(19) United States
(12) Patent Application Publication
(10) Pub. No.: US 2025/0064032 A1
(43) Pub. Date: Feb. 27, 2025

(54) EDITING TMPRSS2/4 FOR DISEASE RESISTANCE IN LIVESTOCK

(71) Applicant: Pig Improvement Company UK Limited, Basingstoke, Hampshire (GB)

(72) Inventors: Andrew Mark Cigan, Madison, WI (US); Benjamin Beaton, DeForest, WI (US); Brian Timothy Burger, Madison, WI (US); Stephen White, Marshall, WI (US)

(73) Assignee: Pig Improvement Company UK Limited, Basingstoke, Hampshire (GB)

(21) Appl. No.: 18/718,117

(22) PCT Filed: Dec. 9, 2022
(86) PCT No.: PCT/GB2022/053152

§ 371 Date: Jun. 10, 2024

Related U.S. Application Data
(60) Provisional application No. 63/265,208, filed on Dec. 10, 2021.

(51) Int. Cl. A01K 67/0275; C12N 9/22; C12N 9/64; C12N 15/11; C12N 15/90

(52) U.S. Cl. CPC A01K 67/0275; C12N 9/22; C12N 9/6424; C12N 15/11; C12N 15/907; A01K 2217/072; A01K 2227/108; C12N 2310/20

(57) ABSTRACT

Livestock animals and progeny thereof comprising at least one modified chromosomal sequence that reduces expression or activity of a Transmembrane Serine Protease 2 (TMPRSS2) protein and/or a Transmembrane Serine Protease 4 (TMPRSS4) protein are provided. Livestock animal cells that contain such edited chromosomal sequences are also provided. The livestock animals, progeny, and cells have increased resistance to influenza viruses and coronaviruses. Methods for producing influenza resistant and porcine epidemic diarrhea resistant livestock animals are also provided.

### CROSS REFERENCE TO RELATED APPLICATION

This application claims the benefit of and priority to U.S. Provisional Application 63/265,208 filed on Dec. 10, 2021. 63/265,208 is hereby incorporated by reference in its entirety.

### SEQUENCE LISTING

The instant application contains a Sequence Listing which has been submitted in ST 26 xml format via EFS-Web and is hereby incorporated by reference in its entirety. Said xml copy, created on Dec. 8, 2022, is named TD-15-2021-WO1-SEQ.xml.

### TECHNICAL FIELD

The present technology relates to gene edited livestock animals and the modification of type II transmembrane serine protease genes to provide disease resistance.

### BACKGROUND

Influenza A viruses (IAVs) are enveloped, single stranded RNA viruses that cause an acute respiratory disease leading to substantial economic losses to the swine industry each year. To date, three major subtypes of IAVs (H1N1, H1N2, and H3N2) have been identified as being endemic in U.S. swine herds.

IAVs are considered to be one of the most important infectious disease agents affecting North American Swine (Sandbulte, M. R., et al., Vaccines, 2015, 3, 22-73). IAVs cause substantial health problems in swine, including high fever, lethargy, anorexia, weight loss, nasal and ocular discharge, cough, sneezing, conjunctivitis, and breathing difficulties (Rajao, D. S., et al., Current Topics in Microbiology and Immunology, 2014, 385, 307-326; CDC.gov “What People Who Raise Pigs Need to Know About Influenza”. 2022; www.cfsph.iastate.edu, “Swine Influenza Technical Factsheet”, 2022). The disease progresses rapidly and may be complicated when associated with other respiratory pathogens, leading to pneumonia and severe clinical signs (Rajao, D. S., et al., Current Topics in Microbiology and Immunology, 2014, 385, 307-326). Swine influenza also causes substantial economic losses as a result of weight loss, reduced weight gain, and reproductive failure in infected sows due to high fevers (Rajao, D. S., et al., Current Topics in Microbiology and Immunology, 2014, 385, 307-326).

Moreover, swine IAVs pose a significant zoonotic threat to humans. Variants of the IAVs that normally infect pigs can emerge and cause disease in humans. For example, in spring of 2009, a new swine-origin H1N1 influenza A virus emerged in Mexico and the United States and spread worldwide by human-to-human transmission (Smith, G. J. D., et al., Nature 2009, 459, 1122-1126). The Centers for Disease Control and Prevention (CDC) estimated that over a one-year period from April 2009 through March 2010, approximately 60 million people were infected, resulting in approximately 12,000 deaths (CDC, CDC Estimates of 2009 H1N1 Cases and Related Hospitalizations and Deaths from April 2009 through Mar. 13, 2010. By Age Group, www(dot)cdc(dot)gov/h1n1flu/pdf/graph_March % 202010.pdf, 2010).

While vaccination of swine represents one strategy for controlling IAV infection, swine IAV strains are very diverse and prone to mutation and vaccines therefore often have disappointing efficacy in the field (Sandbulte, M. R., et al., Vaccines, 2015, 3, 22-73). Furthermore, there has been a dramatic evolutionary expansion in IAV diversity in U.S. swine since 1998, resulting in co-circulation of many antigenically and genetically distinct IAV strains and complicating the control of swine influenza (Rajao, D. S., et al., Current Topics in Microbiology and Immunology, 2014, 385, 307-326; see also www.cfsph.iastate.edu, “Swine Influenza Technical Factsheet”, 2022). Vaccine efficacy has been compromised by this rapid evolution of influenza viruses, resulting in suboptimal protection against distantly related strains (Rajao, D. S., et al., Current Topics in Microbiology and Immunology, 2014, 385, 307-326). Moreover, vaccines eliminate or reduce clinical signs, but do not always prevent infections or virus shedding, though the amount of virus shed may be reduced (CFSPH, 2016). In addition, vaccine-associated enhanced respiratory disease (VAERD), which is characterized by severe respiratory disease, can occur with traditional inactivated vaccines when vaccine virus strains are mismatched with the infecting strain (Rajao, D. S., et al., Current Topics in Microbiology and Immunology, 2014, 385, 307-326).

There is therefore a need in the art for development of additional strategies for the control of IAVs in swine.

Porcine epidemic diarrhea virus (PEDV) is a very important coronavirus-based disease of swine with estimated impact of $900 million-$1.8 billion per year in the U.S. alone (Paarlberg, P., 2014, No. 1240-2016-101641), and there are currently no effective vaccines (Jung, K., et al., Virus Research, 2020, 286, 198045). PEDV is an alpha coronavirus with a noteworthy dependence on proteases for escaping the endosome and achieving entry (Shirato, K., et al., Journal of Virology, 2011, 85, 7872-7880; Liu, C., et al., Journal of Biological Chemistry, 2016, 291, 24779-24786; Oh, C., et al., Virus Research, 2019, 272, 197730). In particular, culture of PEDV field isolates requires addition of protease (Shirato, K., et al., Journal of Virology, 2011, 85, 7872-7880). There is further need in the art for additional strategies for the control of PEDV.

### SUMMARY

Livestock animals and progeny thereof with reduced susceptibility to infection by both an influenza virus and PEDV are provided. The animals and progeny comprise reduced expression or activity of a Transmembrane Serine Protease 2 (TMPRSS2) protein and/or a Transmembrane Serine Protease 4 (TMPRSS4) protein. The livestock animals may comprise at least one modified chromosomal sequence that reduces expression or activity of the TMPRSS2 or TMPRSS4 protein. In some embodiments, the modified chromosomal sequence reduces expression or activity of both the TMPRSS2 protein and the TMPRSS4 protein. In some configurations, the modified chromosomal sequence introduces an exogenous stop codon to TMPRSS2 or TMPRSS4. In some configurations, the modified chromosomal sequence comprises SEQ ID NO: 445 or SEQ ID NO: 446.

Livestock animal cells with reduced susceptibility to infection by an influenza virus are provided. The animal cells comprise reduced expression or activity of a TMPRSS2 and/or TMPRSS4 protein. In some embodiments, the cell is an egg cell or a sperm cell. Fresh or cryopreserved semen inseminate comprising a plurality of sperm cells are provided. A frozen vial, a cell culture, a tissue, a zygote, or an embryo comprising a plurality of the livestock animal cells described herein are also provided.

Methods of generating a livestock animal with reduced susceptibility to infection by an influenza virus are provided. The methods comprise modifying at least one chromosomal sequence in a livestock animal cell so that TMPRSS2 and/or TMPRSS4 protein production or activity is reduced and generating a livestock animal from the cell. Methods of increasing a livestock animal's resistance to infection with an influenza virus are provided. The methods comprise modifying at least one chromosomal sequence so that TMPRSS2 and/or TMPRSS4 protein production or activity is reduced, as compared to TMPRSS2 and/or TMPRSS4 protein production or activity in a livestock animal that does not comprise the modified chromosomal sequence.

In certain embodiments, the present teachings can include a livestock animal comprising a gene edit in a TMPRSS4 gene. In some configurations, the gene edit in the TMPRSS4 gene can encode a TMPRSS4 protein that exhibits reduced cleavage of a viral hemagglutinin (HA) protein and/or a coronavirus spike protein. In various configurations, the gene edit can introduce a premature stop codon. In some configurations, the premature stop codon can be in Exon 5, Exon 9, or Exon 10. In various configurations, the gene edited TMPRSS4 gene can comprise SEQ ID NO: 446. In various configurations, the livestock animal can show increased resistance to a virus relative to a wild type livestock animal. In some configurations, the virus can be an influenza virus or a coronavirus. In various configurations, the virus can be influenza A, influenza D, porcine epidemic diarrhea virus (PEDV), transmissible gastroenteritis coronavirus (TGEV), porcine respiratory coronavirus (PRCV), swine acute diarrhea syndrome coronavirus (SADS-CoV), porcine hemagglutinating encephalomyelitis virus (PHEV), or porcine deltacoronavirus (PDCoV). In various configurations, the virus can be influenza A. In various configurations, the virus can be porcine epidemic diarrhea virus. In various configurations, the livestock animal can be a pig. In various configurations, the livestock animal can be a bovine.

In various configurations, the livestock animal can further comprise a gene edit in a TMPRSS2 gene. In various configurations, the gene edit in the TMPRSS4 gene and the gene edit in the TMPRSS2 gene can each comprise a premature stop codon. In some configurations, the gene edit in the TMPRSS4 gene can comprise a premature stop codon in Exon 5, Exon 9, or Exon 10 and the gene edit in the TMPRSS2 gene can in Exon 3, Exon 5, or Exon 9. In various configurations, the gene edited TMPRSS4 gene can comprise SEQ ID NO: 446 and the gene edit in the TMPRSS2 gene can comprise SEQ ID NO: 445. In various configurations, the livestock animal can show increased resistance to a virus upon infection relative to a wild type livestock animal. In some configurations, the virus can be an influenza virus or a coronavirus. In various configurations, the virus can be influenza A, influenza D, porcine epidemic diarrhea virus (PEDV), transmissible gastroenteritis coronavirus (TGEV), porcine respiratory coronavirus (PRCV), swine acute diarrhea syndrome coronavirus (SADS-COV), porcine hemagglutinating encephalomyelitis virus (PHEV), or porcine deltacoronavirus (PDCOV). In various configurations, the virus can be influenza A. In various configurations, the virus can be porcine epidemic diarrhea virus. In various configurations, the edited TMPRSS2 gene encodes a TMPRSS2 protein that exhibits reduced cleavage of a viral HA protein and/or a viral coronavirus spike protein. In various configurations, the livestock animal can be a pig.

In various embodiments, a livestock animal according to the present teachings can comprise a gene edit in a TMPRSS2 gene. In some configurations, the livestock animal can have increased resistance or decreased susceptibility to a PEDV virus. In various configurations, the gene edit can comprise an edit in the promoter region. In some configurations, the gene edit can be in the TATA box.

In various embodiments, a livestock animal of the present teachings can comprise a gene edit in a TMPRSS2 gene wherein the gene edit can be located downstream of nucleotide 14,361 as compared to SEQ ID NO 437.

In various embodiments, a livestock animal of the present teachings can comprise a gene edit in a TMPRSS2 gene wherein the gene edit affects the sequence of any one of exons 3-16.

In various configurations the livestock animal according to the present teachings can comprise the gene edit in the TMPRSS2 gene in exon 3. In various configurations, the gene edit can comprise a premature stop codon. In various configurations, the gene edited TMPRSS2 gene can encode a TMPRRS2 protein that exhibits reduced cleavage of a viral HA protein and/or a coronavirus spike protein. In various configurations, the premature stop codon can be located in Exon 3, Exon 5, or Exon 9. In various configurations, the gene edit in the TMPRSS2 gene can comprise SEQ ID NO: 445. In various configurations, the livestock animal can be a pig. In various configurations, the livestock animal can show increased resistance to a virus upon infection relative to a wild type livestock animal. In some configurations, the virus can be an influenza virus or a coronavirus. In various configurations, the virus can be influenza A, influenza D, porcine epidemic diarrhea virus (PEDV), transmissible gastroenteritis coronavirus (TGEV), porcine respiratory coronavirus (PRCV), swine acute diarrhea syndrome coronavirus (SADS-COV), porcine hemagglutinating encephalomyelitis virus (PHEV), or porcine deltacoronavirus (PDCoV). In various configurations, the virus can be influenza A. In various configurations, the virus can be porcine epidemic diarrhea virus.

In various embodiments, a method of producing a gene edited livestock animal can comprise: i) introducing a) a Cas9 protein or a nucleic acid encoding a Cas9 protein and b) at least one guide RNA (gRNA) configured to create an edited TMPRSS4 sequence into an isolated livestock animal cell: ii) producing a livestock animal from the isolated livestock animal cell. In some configurations, the edited TMPRSS4 sequence can comprise a premature stop codon. In various configurations, the cell can be a fibroblast cell or a single cell zygote. In some configurations, the edited TMPRSS4 sequence can encode a TMPRSS4 protein that exhibits reduced cleavage of a viral HA protein and/or a coronavirus spike protein. In various configurations, the at least one gRNA can be a pair of gRNAs comprising SEQ ID NOs: 196 and 203, 197 and 202, 208 and 213, 297 and 307, 297 and 312, 328 and 340, 328 and 341, 328 and 343, 319 and 342, 331 and 342, 334 and 342, or 336 and 342. In various configurations, the at least one gRNA can be a pair of gRNAs comprising SEQ ID NOs: 328 and 338. In various configurations, the edited TMPRSS4 sequence can comprise SEQ ID NO: 446. In various configurations, the gene edited livestock animal can be a pig. In various configurations, the method can further comprise introducing at least one gRNA configured to create an edited TMPRSS2 sequence into the livestock animal cell. In various configurations, the method can further comprise mating the gene edited livestock animal to a livestock animal comprising a gene edited TMPRSS2 gene. In various configurations, the method can further comprise making a cell line from the isolated livestock animal cell. In some configurations, the cell line can be a fibroblast cell line.

In various embodiments, a method of producing a gene edited livestock animal can comprise: i) introducing a) a Cas9 protein or a nucleic acid encoding a Cas9 protein and b) at least one guide RNA (gRNA) configured to create an edited TMPRSS2 sequence wherein the edited TMPRSS2 sequence can be downstream of nucleotide 14,361 as compared to SEQ ID NO: 437: ii) producing a livestock animal from the isolated livestock animal cell. In some configurations, the edited TMPRSS2 sequence can encode a TMPRSS2 protein that that exhibits reduced cleavage of a viral HA protein and/or a coronavirus spike protein. In various embodiments, the at least one gRNA can comprise a pair of gRNAs comprising SEQ ID NOs: 10 and 30. In various configurations, the gene edited TMPRSS2 sequence can comprise SEQ ID NO: 445. In various configurations, the gene edited livestock animal can be a pig. In various configurations, the method can further comprise mating the gene edited livestock animal to a livestock animal comprising a gene edited TMPRSS4 gene. In various configurations, the cell can be a fibroblast cell or a single cell zygote. In various configurations, the method can further comprise making a cell line from the isolated livestock animal cell. In some configurations, the cell line can be a fibroblast cell line.

A population of livestock animals that have reduced susceptibility to infection by an influenza virus is provided. The population comprises two or more of any of the livestock animals and/or progeny thereof described herein. Methods of breeding of the livestock animals that have reduced susceptibility to infection by an influenza virus are also provided.

The animals, progeny, cells, populations of animals, and methods are further described herein below.

While multiple embodiments are disclosed, still other embodiments of the inventions will become apparent to those skilled in the art from the following detailed description, which shows and describes illustrative embodiments of the invention. Accordingly, the figures and detailed description are to be regarded as illustrative in nature and not restrictive.

### DETAILED DESCRIPTION

The present disclosure relates to livestock animals and progeny thereof comprising at least one modified chromosomal sequence that reduces expression or activity of a Transmembrane Serine Protease 2 (TMPRSS2) protein and/or a Transmembrane Serine Protease 4 (TMPRSS4) protein. The disclosure further relates to animal cells comprising at least one gene edited chromosomal sequence that reduces expression or activity of a TMPRSS2 and/or a TMPRSS4 protein. The animals and cells have chromosomal modifications or edits (e.g., insertions, deletions, or substitutions) that inactivate or otherwise modulate TMPRSS2 and/or TMPRSS4 expression or activity. In particular, the animals and cells have gene edits that remove a portion of the chromosome such that when the ends are joined during non-homologous end joining (NHEJ), a premature stop codon is introduced within the gene. TMPRSS2 and TMPRSS4 play important roles in viral replication, for example viral replication of influenza viruses or coronaviruses. Without being limited by theory, it is known that viruses such as influenza, SARS-CoV-2 and MERS-CoV utilize TMPRSS2 for viral entry of host cells (Thunders M. and Delahunt B., J. Clin. Pathol., 2020, 73, 773-776). TMPRSS4 has been shown to be involved in viral replication of influenza virus (Ohler, A. and Becker-Pauly, C., Bchm., 2012, 393, 907-914) and enterocyte entry of SARS-CoV-2 (Zang, R., et al., Sci. Immunol., 2020, 5, eabc3582). Porcine epidemic diarrhea virus is also a coronavirus, and may be related to SARS-CoV2. Thus, it is expected that the animals and cells will have increased resistance to viruses, including influenza viruses such as influenza A viruses (IAV) or coronaviruses such as porcine epidemic diarrhea virus (PEDV). The animals and cells can be created using any number of protocols, including those that make use of gene editing.

### Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a”, “an”, and “the” can include plural referents unless the content clearly indicates otherwise. Similarly, the word “or” is intended to include “and” unless the context clearly indicate otherwise. The word “or” means any one member of a particular list and also includes any combination of members of that list.

Units, prefixes, and symbols may be denoted in their SI accepted form. Unless otherwise indicated, nucleic acids are written left to right in 5′ to 3′ orientation: amino acid sequences are written left to right in amino to carboxy orientation, respectively. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 11/2, and 43/4 This applies regardless of the breadth of the range.

The term “about” as used herein, refers to variation in the numerical quantity that can occur, for example, through typical measuring techniques and equipment, with respect to any quantifiable variable, including, but not limited to, mass, volume, time, and temperature. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

A “binding protein” is a protein that is able to bind to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

As used herein “blastocyst” means an early developmental stage of an embryo comprising an inner cell mass (from which the embryo proper arises) and a fluid filled cavity typically surrounded by a single layer of trophoblast cells. “Developmental Biology”, sixth edition, ed. by Scott F. Gilbert, Sinauer Associates, Inc., Publishers, Sunderland, Mass. (2000).

The term “Cas” refers to a “CRISPR associated” protein. Non-limiting examples of Cas proteins include Cas1, Cas1B, Cas2, Cas3, Cas4, Cas5, Cash, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

“Cas9” (formerly referred to as Cas5, Csn1, or Csx12) herein refers to a Cas endonuclease of a type II CRISPR system that forms a complex with a crRNA and a tracrRNA, or with a single guide polynucleotide, for specifically recognizing and cleaving all or part of a DNA target sequence.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains:” “+ and − cleavage half-domains” and “right and left cleavage half-domains” are used interchangeably to refer to pairs of cleavage half-domains that dimerize.

The term “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus.

An “engineered cleavage half-domain” is a cleavage half-domain that has been modified so as to form obligate heterodimers with another cleavage half-domain (e.g., another engineered cleavage half-domain). See, also, U.S. Patent Publication Nos. 2005/0064474, 2007/0218528, 2008/0131962 and 2011/0201055, incorporated herein by reference in their entireties.

As used herein “conditional knock-out” or “conditional mutation” means when the knock-out or mutation is achieved when certain conditions are met. These conditions include, but are not limited to, the presence of certain inducing agents, recombinases, antibiotics, and certain temperature or salt levels.

The term “conservatively modified variants” applies to both amino acid and nucleic acid sequences. With respect to particular nucleic acid sequences, “conservatively modified variants” refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid.

One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to can be so altered. Thus, for example, 1, 2, 3, 4, 5, 7, or 10 alterations can be made.

Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art. The following six groups each contain amino acids that are conservative substitutions for one another: [1] Alanine (A), Serine(S), Threonine (T): [2] Aspartic acid (D), Glutamic acid (E): [3] Asparagine (N), Glutamine (Q): [4] Arginine (R), Lysine (K): [5] Isoleucine (I), Leucine (L), Methionine (M), Valine (V); and [6] Phenylalanine (F), Tyrosine (Y), Tryptophan (W). See also, Creighton (1984) Proteins W. H. Freeman and Company.

The term “early stage embryo” means any embryo at embryonic stages between fertilized ovum and blastocyst. Typically, eight cell stage and morula stage embryos are referred to as early stage embryos.

By “encoding” or “encoded”, with respect to a specified nucleic acid, is meant comprising the information for translation into the specified protein. A nucleic acid encoding a protein may comprise intervening sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. When the nucleic acid is prepared or altered synthetically, advantage can be taken of known codon preferences of the intended host where the nucleic acid is to be expressed.

“Embryonic stem cells” or “ES cells” means cultured cells derived from inner cell mass of early stage embryo, which are amenable to genetic modification and which retain their totipotency and can contribute to all organs of resulting chimeric animal if injected into host embryo. “Developmental Biology”, sixth edition, ed. by Scott F. Gilbert, Sinauer Associates, Inc., Publishers, Sunderland, Mass. (2000).

As used herein, “fertilization” means the union of male and female gametes during reproduction resulting into formation of zygote, the earliest developmental stage of an embryo.

“Exogenous” refers to a nucleic acid sequence originating outside an organism that has been introduced into the organism. This can refer to sequences which naturally occur in a sexually compatible species, sequences which are synthetic, or sequences from another species.

As used herein “full-length sequence” in reference to a specified polynucleotide or its encoded protein means having the entire amino acid sequence of a native (nonsynthetic), endogenous, biologically active form of the specified protein. Methods to determine whether a sequence is full-length are well known in the art including such exemplary techniques as northern or western blots, primer extension, S1 protection, and ribonuclease protection. Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

As used herein, “gene editing” and “gene editing effectors” refer to the use of naturally occurring or artificially engineered nucleases, also referred to as “molecular scissors.” The nucleases create specific double-stranded break (DSBs) at desired locations in the genome, which in some cases harnesses the cell's endogenous mechanisms to repair the induced break by natural processes of homologous recombination (HR) and/or nonhomologous end-joining (NHEJ). Gene editing effectors include Zinc Finger Nucleases (ZFNs), Transcription Activator-Like Effector Nucleases (TALENs), the Clustered Regularly Interspaced Short Palindromic Repeats/CAS (CRISPR/Cas) system, and meganuclease re-engineered as homing endonucleases. The terms also include the use of genetic editing procedures and techniques, including, for example, where the change is relatively small and/or does not introduce DNA from a foreign species.

As used herein, the term “gene edit”, “gene edited”, or “genetically edited” refers to an organism where human intervention, such as but without limitation by using a gene editing effector, has created a genetic difference in its genome when compared to a wild type genome of the same organism. These differences can include but are not limited to nucleotide substitutions, excision of a start codon, or small deletions that do not introduce frame shift mutations into the genome but may excise an exon or form a premature stop codon when the ends of the deletion are ligated together. A gene edit does not introduce DNA from another species into an organism.

A “gene edited animal” refers to an animal with one or more cells comprising a gene edit.

The terms “genome engineering.” “genetic engineering,” “genetically engineered,” “genetically altered,” “genetic alteration,” “genome modification,” “genome modification,” and “genomically modified” can refer to altering the genome by deleting, inserting, mutating, or substituting specific nucleic acid sequences. The altering can be gene or location specific. Genome engineering can use nucleases to cut a nucleic acid thereby generating a site for the alteration. Engineering of non-genomic nucleic acid is also contemplated. A protein containing a nuclease domain can bind and cleave a target nucleic acid by forming a complex with a nucleic acid-targeting nucleic acid. In one example, the cleavage can introduce double-stranded breaks in the target nucleic acid. A nucleic acid can be repaired e.g., by endogenous non-homologous end joining (NHEJ) machinery. In a further example, a piece of nucleic acid can be inserted. Modifications of nucleic acid-targeting nucleic acids and site-directed polypeptides can introduce new functions to be used for genome engineering.

As used herein, the phrase “gene edit affects the sequence of” may refer to an edit that affects direct change in a sequence, or that affects a change in a nearby sequence that affects the structure of the sequence, for example but without limitation, a change in an intron sequence that leads to a splice variant or other change in the protein encoded by the edited DNA.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

As used herein “homing DNA technology” or “homing technology” covers any mechanisms that allow a specified molecule to be targeted to a specified DNA sequence including Zinc Finger (ZF) proteins, Transcription Activator-Like Effectors (TALEs) meganucleases, and CRISPR systems (e.g., CRISPR/Cas9 systems).

The terms “increased resistance” and “reduced susceptibility” herein mean, but are not limited to, a statistically significant reduction of the incidence and/or severity of clinical signs or clinical symptoms which are associated with infection by pathogen. For example, “increased resistance” or “reduced susceptibility” can refer to a statistically significant reduction of the incidence and/or severity of clinical signs or clinical symptoms which are associated with infection by an influenza virus or a coronavirus such as porcine epidemic diarrhea virus in an animal comprising a modified chromosomal sequence as compared to a control animal having an unmodified chromosomal sequence. The terms “increased resistance” and “reduced susceptibility” can also denote lower pathogen replication upon infection, such as for an influenza virus or a coronavirus such as porcine epidemic diarrhea virus. The term “statistically significant reduction of clinical symptoms” means, but is not limited to, the frequency in the incidence of at least one clinical symptom in the modified group of subjects is at least 10%, preferably at least 20%, more preferably at least 30%, even more preferably at least 50%, and even more preferably at least 70% lower than in the non-modified control group after the challenge with the infectious agent. As used herein, clinical signs can include, but are not limited to, measures of viral load in an animal or organ thereof. As used herein, clinical symptoms can include, but are not limited to, symptoms that cannot be observed without invasive tests or procedures, such as, but without limitation, lesions in the lungs.

The term “isolated” refers to material, such as a nucleic acid or a protein, which is: (1) substantially or essentially free from components that normally accompany or interact with it as found in its naturally occurring environment—the isolated material optionally comprises material not found with the material in its natural environment: or (2) if the material is in its natural environment, the material has been synthetically altered by deliberate human intervention to a composition and/or placed at a location in the cell (e.g., genome or subcellular organelle) not native that material. The alteration to yield the synthetic material can be performed on the material either within or removed from its natural state. For example, a naturally occurring nucleic acid becomes an isolated nucleic acid if it is altered, or if it is transcribed from DNA which has been altered, by means of human intervention performed within the cell from which it originates. See. e.g., Compounds and Methods for Site Directed Mutagenesis in Eukaryotic Cells, Kmiec, U. S. U.S. Pat. No. 5,565,350: In Vivo Homologous Sequence Targeting in Eukaryotic Cells: Zarling et al., PCT/US93/03868. Likewise, a naturally occurring nucleic acid (e.g., a promoter) becomes isolated if it is introduced by non-naturally occurring means to a locus of the genome not native to that nucleic acid. Nucleic acids which are “isolated” as defined herein, may also be referred to as “heterologous” nucleic acids.

“Knock-out” means disruption of the structure or regulatory mechanism of a gene. Knock-outs may be generated through homologous recombination of targeting vectors, replacement vectors or hit-and-run vectors or random insertion of a gene trap vector resulting into complete, partial or conditional loss of gene function.

The term “livestock animal” includes any animals traditionally raised in livestock farming, for example an ungulate (e.g., an artiodactyl), an avian animal (e.g., chickens, turkeys, ducks, geese, guinea fowl, or squabs), an equine animal (e.g., horses or donkeys). Ungulates include, but are not limited to porcine animals (e.g., pigs), bovine animals (e.g., beef or dairy cattle, buffalo), ovine animals, caprine animals, camels, llamas, alpacas, and deer. The term does not include rats, mice, or other rodents.

As used herein, “nucleic acid” includes reference to a deoxyribonucleotide or ribonucleotide polymer in either single- or double-stranded form, and unless otherwise limited, encompasses conservatively modified variants and known analogues having the essential nature of natural nucleotides in that they hybridize to single-stranded nucleic acids in a manner similar to naturally occurring nucleotides (e.g., peptide nucleic acids).

The term “mutation” includes alterations in the nucleotide sequence of a polynucleotide, such as for example a gene or coding DNA sequence (CDS), compared to the wild-type sequence. The term includes, without limitation, substitutions, insertions, frameshifts, deletions, inversions, translocations, duplications, splice-donor site mutations, point-mutations and the like.

The terms “polypeptide”, “peptide” and “protein” are used interchangeably herein to refer to a polymer of amino acid residues. The terms also may apply to conservatively modified variants and to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, the protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not always entirely linear. For instance, polypeptides may be branched as a result of ubiquitization, and they may be circular, with or without branching, generally as a result of post-translation events, including natural processing events and events brought about by human manipulation which do not occur naturally. Circular, branched, and branched circular polypeptides may be synthesized by non-translation natural process and by entirely synthetic methods. Further, this invention contemplates the use of both the methionine-containing and the methionine-less amino terminal variants of the protein of the invention.

As used herein “promoter” includes reference to a region of DNA upstream from the start of transcription and involved in recognition and binding of RNA polymerase and other proteins to initiate transcription. Examples of promoters under developmental control include promoters that preferentially initiate transcription in certain tissues, such as testes, ovaries, or placenta. Such promoters are referred to as “tissue-preferred”. Promoters which initiate transcription only in certain tissue are referred to as “tissue-specific”. A “cell type” specific promoter primarily drives expression in certain cell types in one or more organs, for example, germ cells in testes or ovaries. An “inducible” or “repressible” promoter is a promoter which is under environmental control. Examples of environmental conditions that may affect transcription by inducible promoters include stress, and temperature. Tissue-specific, tissue-preferred, cell-type specific and inducible promoters constitute the class of “non-constitutive” promoters. A “constitutive” promoter is a promoter which is active under most environmental conditions.

The terms “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

“Resistance” of an animal to a disease is a characteristic of an animal, wherein the animal avoids the disease symptoms that are the outcome of animal-pathogen interactions, such as interactions between a porcine animal and an influenza virus or a coronavirus. That is, pathogens are prevented from causing animal diseases and the associated disease symptoms, or alternatively, a reduction of the incidence and/or severity of clinical signs or reduction of clinical symptoms. One of skill in the art will appreciate that the methods disclosed herein can be used with other compositions and methods available in the art for protecting animals from pathogens.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Zinc finger and TALE binding domains can be “engineered” to bind to a predetermined nucleotide sequence, for example via engineering (altering one or more amino acids) of the recognition helix region of naturally occurring zinc finger or TALE proteins. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058: WO 98/53059: WO 98/53060: WO 02/016536 and WO 03/016496 and U.S. Publication No. 20110301073.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “selected” zinc finger protein or TALE is a protein not found in nature whose production results primarily from an empirical process such as phage display, interaction trap or hybrid selection. See e.g., U.S. Pat. Nos. 5,789,538; 5,925,523; 6,007,988; 6,013,453; 6,200,759; WO 95/19431: WO 96/06166: WO 98/53057: WO 98/54311: WO 00/27878: WO 01/60970 WO 01/88197, WO 02/099084 and U.S. Publication No. 20110301073.

“Wild type” means those animals and blastocysts, embryos or cells derived therefrom, which have not been genetically modified and are usually inbred and outbred strains developed from naturally occurring strains.

The following terms are used to describe the sequence relationships between a polynucleotide/polypeptide of the present invention with a reference polynucleotide/polypeptide: (a) “reference sequence”, (b) “comparison window”, (c) “sequence identity”, and (d) “percentage of sequence identity”.
(a) As used herein, “reference sequence” is a defined sequence used as a basis for sequence comparison with a polynucleotide/polypeptide of the present invention. A reference sequence may be a subset or the entirety of a specified sequence: for example, as a segment of a full-length cDNA or gene sequence, or the complete cDNA or gene sequence.
(b) As used herein, “comparison window” includes reference to a contiguous and specified segment of a polynucleotide/polypeptide sequence, wherein the polynucleotide/polypeptide sequence may be compared to a reference sequence and wherein the portion of the polynucleotide/polypeptide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides/amino acids residues in length, and optionally can be 30, 40, 50, 100, or longer. Those of skill in the art understand that to avoid a high similarity to a reference sequence due to inclusion of gaps in the polynucleotide/polypeptide sequence, a gap penalty is typically introduced and is subtracted from the number of matches.

Methods of alignment of sequences for comparison are well-known in the art. Optimal alignment of sequences for comparison may be conducted by the local homology algorithm of Smith, T. F. and Waterman, M. S., Adv. Appl. Math., 1981, 2, 482-489; by the homology alignment algorithm of Needleman, S. B. and Wunsch, C. D., J. Mol. Biol., 48, 1970, 443-453; by the search for similarity method of Pearson, W. R. and Lipman, D. J., Proc. Natl. Acad. Sci., 1988, 85, 2444-2448; and by computerized implementations of these algorithms, including, but not limited to: CLUSTAL in the PC/Gene program by Intelligenetics, Mountain View, California: GAP, BESTFIT, BLAST, FASTA, and TFASTA, and related programs in the GCG Wisconsin Genetics Software Package, Version (available from Accelrys Inc., 9685 Scranton Road, San Diego, California, USA). The CLUSTAL program is well described by Higgins, D. G. and Sharp, P. M., Gene, 1988, 73:237-244: Higgins, D. G. and Sharp, P. M., CABIOS, 1989, 5, 151-153: Corpet, F., Nucleic Acids Research, 1988, 16, 10881-10890; Huang, X., et al., Computer Applications in the Biosciences, 1992, 8, 155-65, and Pearson, W. R., Methods in Molecular Biology, 1994, 24, 307-331.

The BLAST family of programs that can be used for database similarity searches includes: BLASTN for nucleotide query sequences against nucleotide database sequences: BLASTX for nucleotide query sequences against protein database sequences: BLASTP for protein query sequences against protein database sequences: TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995): Altschul, S. F., et al., J. Mol. Biol., 1990, 215, 403-410; and, Altschul, S. F., et al., Nucleic Acids Res., 1997, 25, 3389-3402. Software for performing BLAST analyses is publicly available, for example through the National Center for Biotechnology Information (ncbi.nlm.nih.gov/). This algorithm has been thoroughly described in a number of publications. See. e.g., Altschul, S. F., et al., Nucleic Acids Res., 1997, 25, 3389-3402: National Center for Biotechnology Information, THE NCBI HANDBOOK [INTERNET], Chapter 16: The BLAST Sequence Analysis Tool (McEntyre J, Ostell J, eds., 2002), available at www(dot)ncbi(dot)nlm(dot)nih(dot)gov/books/NBK21097/pdf/ch16.pdf. The BLASTP program for amino acid sequences has also been thoroughly described (see Henikoff, S. & Henikoff, J. G., Proc. Natl. Acad. Sci. USA, 1989, 89, 10915-10919). In addition to calculating percent sequence identity, the BLAST algorithm also performs a statistical analysis of the similarity between two sequences (see. e.g., Karlin, S. & Altschul. S. F., Proc. Nat'l. Acad. Sci. USA, 1993, 90:5873-5877). A number of low-complexity filter programs can be employed to reduce such low-complexity alignments. For example, the SEG (Wooten, J. C. and Federhen, S., Comput. Chem., 1993, 17, 149-163) and XNU (Claverie, J. M. and States, D. J., Comput. Chem., 1993, 17:191-201) low-complexity filters can be employed alone or in combination.

Unless otherwise stated, nucleotide and protein identity/similarity values provided herein are calculated using GAP (GCG Version 10) under default values. GAP (Global Alignment Program) can also be used to compare a polynucleotide or polypeptide of the present invention with a reference sequence. GAP uses the algorithm of Needleman, S. B. and Wunsch, C. D., J. Mol. Biol., 48, 1970, 443-453 to find the alignment of two complete sequences that maximizes the number of matches and minimizes the number of gaps. GAP represents one member of the family of best alignments. There may be many members of this family, but no other member has a better quality. GAP displays four figures of merit for alignments: Quality; Ratio, Identity, and Similarity: The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff, S. & Henikoff, J. G., Proc. Natl. Acad. Sci. USA, 1989, 89, 10915-10919).

Multiple alignment of the sequences can be performed using the CLUSTAL method of alignment (Higgins, D. G. and Sharp, P. M., CABIOS, 1989, 5, 151-153) with the default parameters (GAPPENALTY=10, GAP LENGTH PENALTY=10). Default parameters for pairwise alignments using the CLUSTAL method include KTUPLE 1, GAP PENALTY=3, WINDOW=5 and DIAGONALS SAVED=5.
(c) As used herein, “sequence identity” or “identity” in the context of two nucleic acid or polypeptide sequences includes reference to the residues in the two sequences which are the same when aligned for maximum correspondence over a specified comparison window: When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions may be calculated according to the algorithm of Meyers, E. W., and Miller, W., Computer Applic. Biol. Sci., 1988, 4, 11-17, for example as implemented in the program PC/GENE (Intelligenetics, Mountain View; California, USA).
(d) As used herein, “percentage of sequence identity” means the value determined by comparing two optimally aligned sequences over a comparison window; wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

### TMPRSS2 and TMPRSS4 Genes

The type II transmembrane serine proteases (TTSPs), TMPRSS2 and TMPRSS4, play important roles in viral replication, supporting the spread of virus in the absence of trypsin. Viral replication supported by TMPRSS2 and TMPRSS4 include influenza replication and coronavirus replication. TMPRSS2 and TMPRSS4 have been evaluated for their role in hemagglutinin (HA) cleavage, which is important for the infectivity of IAVs. The same activity may be important for a range of other influenza viruses, including but not limited to influenza D (Yu, J., et al., Journal of General Virology, 2021, 102, jgv001529). Additionally, TMPRSS proteins are known to cleave spike proteins of coronaviruses such as, but without limitation PEDV transmissible gastroenteritis coronavirus (TGEV), porcine respiratory coronavirus (PRCV), swine acute diarrhea syndrome coronavirus (SADS-COV), porcine hemagglutinating encephalomyelitis virus (PHEV), or porcine deltacoronavirus (PDCOV) (Turlewicz-Podbielska, H and Pomorska-Mól, M, Virologica Sinica, 2021, 36, 833-851), as well as bovine coronavirus (Vlasova A. N. and Saif, L. J., Frontiers in Veterinary Science, 2021, 8, 643220). These and additional newly emerging coronaviruses are of concern. TMPRSS2 activates HA in the mouse and human respiratory tract, and it is hypothesized that TMPRSS2 activates HA in the porcine respiratory tract. Tmprss2−/−, Tmprss4−/−double-knockout mice demonstrated reduced mortality after infection with influenza A virus.

The full-length porcine TMPRSS2 gene (Chromosome 13:204,876,561-204,902,561 reverse strand) is about 26,000 base pairs long. A nucleotide sequence for full-length wild-type porcine TMPRSS2 (SEQ ID NO: 437) is provided. SEQ ID NO: 437 includes 2000 base pairs of sequence upstream of the TMPRSS2 gene which is believed to contain upstream regulatory sequences including the promoter: this 2000 base pair domain is referred to herein as the promoter region an is set forth in SEQ ID NO:437, base pairs 1-2000. Predicted transcripts for full length TMPRSS proteins vary by the database used by the skilled artisan. The NCBI predicted amino acid sequence is set forth in SEQ ID NO: 438 (NCBI protein NP_001373060.1, NCBI transcript NM_001386131.1, 496 amino acids). The Genbank predicted amino acid sequence is set forth in SEQ ID NO: 439 (Genbank BAF76737.1, 495 amino acids). The predicted Ensembl transcript is set forth in SEQ ID NO: 440 (Ensembl transcript ENSSSCT00000026685.4, 495 amino acids). The 5′ gene structure is set forth in Table 1.

TABLE 1
TMPRSS2 Coding Sequence

| Feature | Coordinates in SEQ ID NO: 437 |
|---|---|
| Promoter Region | 1-2000 |
| Exon 1 | 2001-2089 |
| Intron 1 | 2090-11,344 |
| Exon 2 | 11,345-11,415 |
| Intron 2 | 11,416-14,360 |
| Exon 3 | 14,361-14,589 |
| Intron 3 | 14,590-20,954 |
| Exon 4 | 20,955-21,041 |
| Intron 4 | 21,042-21,948 |
| Exon 5 | 21,949-22,071 |
| Intron 5 | 22,072-26,767 |
| Exon 6 | 26,768-26,894 |
| Intron 6 | 26,895-28,541 |
| Exon 7 | 28,542-28,652 |
| Intron 7 | 28,653-32,071 |
| Exon 8 | 32,072-32,115 |
| Intron 8 | 32,116-33,102 |
| Exon 9 | 33,103-33,277 |
| Intron 9 | 33,278-34,542 |
| Exon 10 | 24,543-34,570 |
| Intron 10 | 34,571-34,572 |
| Exon 11 | 34,573-34,717 |
| Intron 11 | 34,718-35,388 |
| Exon 12 | 35,389-35,484 |
| Intron 12 | 35,485-37,966 |
| Exon 13 | 37,967-38,109 |
| Intron 13 | 38,110-38,526 |
| Exon 14 | 38,527-38,679 |
| Intron 14 | 38,680-40,256 |
| Exon 15 | 40,257-41,292 |
| Intron 15 | 41,293-41,325 |
| Exon 16 | 41,326-41,727 |

The coding regions for all splice variants start with 5 amino acids in Exon 2. Exon 1 contains upstream non-coding region. There are 16 total exons in the Ensemble transcript.

The full-length porcine TMPRSS4 gene is about 35,400 base pairs long and encodes at least three splice variants. A nucleotide sequence for full-length wild-type porcine TMPRSS4 (SEQ ID NO: 441) is provided, as are predicted amino acid sequences for the presumptive full-length wild-type porcine TMPRSS4 protein encoded by splice variant 201 (435 amino acids, 13 exons; SEQ ID NO: 442), splice variant 202 (397 amino acids, 12 exons; SEQ ID NO: 443), and splice variant 203 (435 amino acids; SEQ ID NO: 444, 13 exons). Splice variant is 203 is associated with Gene ID NO: 100514419 in NCBI and Ensembl ID ENSSSCG00000015086.4 and transcript ID ENSSSCT00000040038.2. The gene structure for this transcript is listed in Table 2.

TABLE 2
TMPRSS4 Coding Sequence

| | Variant 201 | Variant 202 | Variant 203 |
|---|---|---|---|
| Exon | Nucleotides in | Nucleotides in | Nucleotides in |
| Number | SEQ ID NO: 441 | SEQ ID NO: 441 | SEQ ID NO: 441 |
| 1 | 367 . . . 369 | 367 . . . 369 | 137 . . . 369 |
| 2 | 13,759 . . . 13,792 | 13,759 . . . 13,792 | 13,759 . . . 13,792 |
| 3 | 18,135 . . . 18,248 | 21,081 . . . 21,233 | 18,135 . . . 18,248 |
| 4 | 21,081 . . . 21,233 | 22,826 . . . 22,955 | 21,081 . . . 21,233 |
| 5 | 22,826 . . . 22,955 | 25,640 . . . 25,741 | 22,826 . . . 22,955 |
| 6 | 25,640 . . . 25,741 | 26,688 . . . 26,728 | 25,640 . . . 25,741 |
| 7 | 26,688 . . . 26,728 | 29,191 . . . 29,350 | 26,688 . . . 26,728 |
| 8 | 29,191 . . . 29,350 | 30,381 . . . 30,547 | 29,191 . . . 29,350 |
| 9 | 30,381 . . . 30,547 | 31,264 . . . 31,362 | 30,381 . . . 30,547 |
| 10 | 31,264 . . . 31,362 | 31,549 . . . 31,691 | 31,264 . . . 31,362 |
| 11 | 31,549 . . . 31,691 | 33,044 . . . 33,193 | 31,549 . . . 31,691 |
| 12 | 33,044 . . . 33,193 | 33,793 . . . 33,801 | 33,044 . . . 33,193 |
| 13 | 33,793 . . . 33,801 | — | 34,067 . . . 34,078 |

### TMPRSS2 and TMPRSS4 Gene Editing

The present disclosure provides a livestock animal or animal cell, including sperm or egg cells, with improved resistance to viral infections such as influenza virus infections or coronavirus infections. The livestock animals or cells comprise altered expression of a TMPRSS2 and/or TMPRSS4 protein or other TTSPs associated with the infectivity of influenza virus and/or coronavirus. The livestock animals or cells can comprise at least one modified chromosomal sequence that reduces expression or activity of the TMPRSS2 and/or TMPRSS4 protein. The chromosomal sequence may be (1) inactivated, (2) modified, or (3) comprise an integrated sequence. An inactivated chromosomal sequence is altered such that TMPRSS2 and/or TMPRSS4 protein function is impaired, reduced or eliminated. As used herein, reduced TMPRSS2 or TMPRSS4 protein function or activity (e.g., serine protease activity) refers to a reduction in protein function or activity relative to the function of a wild type TMPRSS2 or TMPRSS4 protein. Thus, a genetically modified animal comprising an inactivated chromosomal sequence may be termed a “knock-out” or a “conditional knock-out”. Similarly, a genetically modified animal comprising an integrated sequence may be termed a “knock in” or a “conditional knock in”. Furthermore, a genetically modified animal comprising a modified chromosomal sequence may comprise a targeted point edit(s) or other modification such that an altered protein product is produced. Briefly, the process can comprise using a CRISPR system (e.g., a CRISPR/Cas9 system) to modify the genomic sequence. To use Cas9 to modify genomic sequences, the protein can be delivered directly to a cell. Alternatively, an mRNA that encodes Cas9 can be delivered to a cell, or a gene that provides for expression of an mRNA that encodes Cas9 can be delivered to a cell. In addition, either target specific crRNA and a tracrRNA can be delivered directly to a cell or target specific sgRNA(s) can be delivered to a cell (these RNAs can alternatively be produced by a gene constructed to express these RNAs). The process of editing chromosomal sequences using a CRISPR system is rapid, precise, and highly efficient.

In some embodiments, a TMPRSS2 and/or TMPRSS4 locus is used as a target site for the site-specific editing. This can include insertion of an exogenous nucleic acid (e.g., a nucleic acid comprising a nucleotide sequence encoding a polypeptide of interest) or deletions of nucleic acids from the locus. In particular embodiments, insertions and/or deletions result in a modified locus. For example, integration of the exogenous nucleic acid and/or deletion of part of the genomic nucleic acid may modify the locus so as to produce a disrupted (i.e., inactivated) TMPRSS2 and/or TMPRSS4 gene.

Any of the animals or cells can be an animal or cell that has been genetically modified using a Clustered Regularly Interspaced Short Palindromic Repeats (CRISPR)/Cas system. The CRISPR/Cas system can suitably comprise any of the guide RNAs (gRNAs) described herein.

For any of the animals, progeny, or cells, the modified chromosomal sequence can be a modified chromosomal sequence that has been produced via homology directed repair (HDR). Alternatively, the modified chromosomal sequence can be a modified chromosomal sequence that has been produced via non-homologous end-joining (NEHJ).

The modified chromosomal sequence reduces the susceptibility of the animal, progeny, or cell to infection by an influenza virus or a coronavirus, as compared to the susceptibility of a livestock animal, progeny, or cell that does not comprise the modified chromosomal sequence. The modified chromosomal sequence preferably substantially eliminates susceptibility of the animal, progeny, or cell to the influenza virus or coronavirus. The modified chromosomal sequence more preferably completely eliminates susceptibility of the animal, progeny, or cell to the influenza virus or coronavirus, such that animals do not show any clinical signs of disease following exposure to the influenza virus or coronavirus. For example, porcine animals having the modified chromosomal sequence do not show any clinical signs of influenza (e.g., fever, lethargy, anorexia, weight loss, nasal and ocular discharge, cough, sneezing, conjunctivitis, breathing difficulties, and/or lung lesions) following exposure to the influenza virus. In addition, in porcine animals having the modified chromosomal sequence, influenza or coronavirus nucleic acid cannot be detected in the nasal secretions, feces, or serum: nor can influenza or coronavirus antigen be detected in the tissues of the animal (e.g., in lung tissue). Further, serum is negative for influenza-specific antibody or coronavirus-specific antibody. Similarly, cells having the modified chromosomal sequence that are exposed to the pathogen do not become infected with the pathogen.

Alternatively, porcine animals having the modified chromosomal sequence may show reduced clinical signs of influenza (e.g., fever, lethargy, anorexia, weight loss, nasal and ocular discharge, cough, sneezing, conjunctivitis, breathing difficulties, and/or lung lesions) and reduced viral titers in the lungs.

The influenza virus preferably comprises a type A influenza virus. The type A influenza virus can be an H1N1, H1N2 or H3N2 type A influenza virus.

Alternatively or in addition, porcine animals having the modified chromosome may have reduced clinical signs or symptoms of PEDV (e.g. diarrhea, excessive scouring, vomiting, dehydration, or increased infant mortality). Preferably, porcine animals would show resistance to PEDV infection.

The coronavirus can be any coronavirus that infects pigs: PEDV is of particular concern.

The modified chromosomal sequence can comprise an insertion, a deletion, a substitution, or a combination of any thereof. In certain embodiments, the edit can disrupt the promoter region of the TMPRSS2 and/or TMPRSS4 gene so as to reduce or eliminate the expression of the TMPRSS2 and/or TMPRSS4 template. Edits that disrupt the promoter region can include edits that remove or otherwise destroy the TATA box from the promoter region. Alternatively or in addition, the insertion, the deletion, the substitution, or the combination of any thereof can result in a miscoding in the allele of the gene encoding the TMPRSS2 and/or TMPRSS4 protein. Where the insertion, the deletion, the substitution, or the combination of any thereof results in a miscoding in the allele of the gene encoding the TMPRSS2 and/or TMPRSS4 protein, the miscoding can result in a premature stop codon in the allele of the gene encoding the TMPRSS2 and/or TMPRSS4 protein. In some embodiments, editing strategies are designed to introduce premature stop codons in conserved exonic sequences of TMPRSS2 and TMPRSS4. In certain embodiments, premature stop codons are introduced either early in the coding sequence or in coding regions containing protease active site residues. In some configurations, these editing strategies include the use of two guides to cut the chromosome and then, through non-homologous end joining (NHEJ) create an exogenous stop codon when the cut ends are ligated.

TABLE 3
Exemplary gRNAs

| gRNA | Target | Sequence |
|---|---|---|
| SEQ ID NO: 10 | TMPRSS2 | cacccgccgtcgtcgtcagcagg |
| SEQ ID NO: 30 | TMPRSS2 | caggccctatggggcgtatccgg |
| SEQ ID NO: 196 | TMPRSS4 | acaaatactacttcctctgctgg |
| SEQ ID NO: 197 | TMPRSS4 | agtggaggggctgccagcagagg |
| SEQ ID NO: 202 | TMPRSS4 | cgtcacacagctgctctctcggg |
| SEQ ID NO: 203 | TMPRSS4 | ccgagagagcagctgtgtgacgg |
| SEQ ID NO: 208 | TMPRSS4 | ccagcaggactgtgcctcagggg |
| SEQ ID NO: 213 | TMPRSS4 | gtgtcaagaaattccccgatggg |
| SEQ ID NO: 297 | TMPRSS4 | agatgtgctgtttgttgtactgg |
| SEQ ID NO: 307 | TMPRSS4 | ggctgccgtgaggatccagtggg |
| SEQ ID NO: 312 | TMPRSS4 | tgggccttaccagaagcagtggg |
| SEQ ID NO: 319 | TMPRSS4 | agcatctcgatgtgcccaactgg |
| SEQ ID NO: 328 | TMPRSS4 | gttgcccagtttgtctgagccgg |
| SEQ ID NO: 334 | TMPRSS4 | gtgatgaagatcttggccacagg |
| SEQ ID NO: 336 | TMPRSS4 | tctttgggggacgtgctgttggg |
| SEQ ID NO: 340 | TMPRSS4 | agggcaatgtctttctctttggg |
| SEQ ID NO: 341 | TMPRSS4 | aagggcaatgtctttctctttgg |
| SEQ ID NO: 342 | TMPRSS4 | cgggagctgcagcttcacaaggg |
| SEQ ID NO: 343 | TMPRSS4 | gcgggagctgcagcttcacaagg |

Guides that use this strategy include SEQ ID NOs: 10 and 30 (for TMPRSS2) and SEQ ID NOs: 196 and 203, 197 and 202, 208 and 213, 297 and 307, 297 and 312, 328 and 340, 328 and 341, 328 and 343, 319 and 342, 331 and 342, 334 and 342, and 336 and 342 (for TMPRSS4). The guide sequences cacccgccgtcgtcgtcagcagg (SEQ ID NO: 10) and caggccctatggggcgtatccgg (SEQ ID NO: 30) create an exogenous stop codon in TMPRSS2, wherein the edited sequence comprises aagagcgaaggcagacccggaactcgggttagagagagaaacacaaaaaggtcctacttgaagagcacatcgtcctggatcgg ggtttgggctgcctgctacgccccatagggcctgtgggccccggggggctgcggggggtagacactttcaggttggtatccgtgg ttttcatagtaaggcccgacacctggccgcga (SEQ ID NO: 445). The guide sequences gttgcccagtttgtctgagccgg (SEQ ID NO: 328) and ggcaatgtctttctctttggggg (SEQ ID NO: 338) create an exogenous stop codon in TMPRSS4, wherein the edited sequence comprises ctgcgcacccagcctcctcatcttgcctcagacctgaaccccactgtctctgcccccaggaagcatctcgatgtgcccaactggaa ggtgagggccggctaagagaaagacattgcccttgtgaagctgcagctcccgctcacgttctccggtgagagacggcctccctgc cgtcagggagggctcaggc cctgggcata (SEQ ID NO: 446).

In some configurations, guides directed to both TMPRSS2 and TMPRSS4 can be introduced into the same cell to edit both genes in the same animal. In various configurations, the traits can be combined by mating a TMPRSS2 gene edited animal to a TMPRSS4 gene edited animal using routine husbandry practices (including AI, IFV, or natural service).

In any of the animals, progeny, or cells described herein, the modified chromosomal sequence preferably causes TMPRSS2 and/or TMPRSS4 protein production or activity to be reduced, as compared to TMPRSS2 and/or TMPRSS4 protein production or activity in an animal, progeny, or cell that lacks the modified chromosomal sequence.

Preferably, the modified chromosomal sequence results in production of substantially no functional TMPRSS2 and/or TMPRSS4 protein by the animal, progeny, or cell. By “substantially no functional TMPRSS2 and/or TMPRSS4 protein,” it is meant that the level of TMPRSS2 and/or TMPRSS4 protein in the animal, progeny, or cell is undetectable, or if detectable, is at least about 90% lower, preferably at least about 95% lower, more preferably at least about 98%, lower, and even more preferably at least about 99% lower than the level observed in an animal, progeny, or cell that does not comprise the modified chromosomal sequences. For any of the animals, progeny, or cells described herein, the animal, progeny, or cell preferably does not produce TMPRSS2 and/or TMPRSS4 protein.

The TMPRSS2 and/or TMPRSS4 gene in the animal, progeny, or cell can comprise any combination of any of the edited chromosomal sequences described herein. Additionally, the edited sequences may be combined or “stacked” with other traits, including other gene edits to create animals comprising multiple desirable traits. Potential stackable edits include edits in ANP32, ANPEP, TMPRSS1, CD163, Melanocortin-4 receptor (MC4R), HMGA, IGF2, E. coli F4ab/ac, HAL, RN, Mx1, BAT2, EHMT2, AMPK, PKM2, PDH, LDHA, LDHC, and ESR. Exemplary CD163 edits are disclosed in WO/2021/224599.

The edited chromosomal sequence can comprise an edit in Exon 1 of TMPRSS2. The edited chromosomal sequence can comprise an edit in Exon 3 of TMPRSS2. The edited chromosomal sequence can comprise an edit in Exon 4 of TMPRSS2. The edited chromosomal sequence can comprise an edit in Exon 5 of TMPRSS2. The edited chromosomal sequence can comprise an edit in Exon 6 of TMPRSS2. The edited chromosomal sequence can comprise an edit in Exon 7 of TMPRSS2. The edited chromosomal sequence can comprise an edit in Exon 8 of TMPRSS2. The edited chromosomal sequence can comprise an edit in Exon 9 of TMPRSS2. The edited chromosomal sequence can comprise an edit in Exon 10 of TMPRSS2. The edited chromosomal sequence can comprise an edit in Exon 11 of TMPRSS2. The edited chromosomal sequence can comprise an edit in Exon 12 of TMPRSS2. The edited chromosomal sequence can comprise an edit in Exon 13 of TMPRSS2. The edited chromosomal sequence can comprise an edit in Exon 14 of TMPRSS2. The edited chromosomal sequence can comprise an edit in Exon 15 of TMPRSS2. The edited chromosomal sequence can comprise an edit in Exon 116 of TMPRSS2.

The edited chromosomal sequence can comprise an edit in Exon 1 of TMPRSS4. The edited chromosomal sequence can comprise an edit in Exon 2 of TMPRSS4. The edited chromosomal sequence can comprise an edit in Exon 3 of TMPRSS4. The edited chromosomal sequence can comprise an edit in Exon 4 of TMPRSS4. The edited chromosomal sequence can comprise an edit within the region comprising nucleotides 22,826 through 22,955 of reference sequence SEQ ID NO: 441. The edited chromosomal sequence can comprise an edit in Exon 5 of TMPRSS4. The edited chromosomal sequence can comprise an edit in Exon 6 of TMPRSS4. The edited chromosomal sequence can comprise an edit in Exon 7 of TMPRSS4. The edited chromosomal sequence can comprise an edit within the region comprising nucleotides 30,381 through 30,547 of reference sequence SEQ ID NO: 441. The edited chromosomal sequence can comprise an edit in Exon 8 of TMPRSS4. The edited chromosomal sequence can comprise an edit in Exon 9 of TMPRSS4. The edited chromosomal sequence can comprise an edit in Exon 10 of TMPRSS4. The edited chromosomal sequence can comprise an edit in Exon 11 of TMPRSS4. The edited chromosomal sequence can comprise an edit in Exon 12 of TMPRSS4. The edited chromosomal sequence can comprise an edit in Exon 13 of TMPRSS4.

In any of the animals, progeny, or cells described herein, the animal, progeny, or cell can comprise a chromosomal sequence in the gene encoding the TMPRSS2 protein having at least 85%, at least 90%, at least 95%, at least 98%, at least 99%, at least 99.9%, or 100% sequence identity to SEQ ID NO: 437 in the regions of the chromosomal sequence outside of the insertion, the deletion, or the substitution.

In any of the animals, progeny, or cells described herein, the animal, progeny, or cell can comprise a chromosomal sequence in the gene encoding the TMPRSS4 protein having at least 85%, at least 90%, at least 95%, at least 98%, at least 99%, at least 99.9%, or 100% sequence identity to SEQ ID NO: 441 in the regions of the chromosomal sequence outside of the insertion, the deletion, or the substitution.

### Guide RNAs

Guide RNAs (gRNAs) are provided. The gRNAs have a nucleic acid sequence that is complementary to a sequence of a gene encoding a TMPRSS2 or TMPRSS4 protein and can be used to introduce a chromosomal modification into a gene encoding a TMPRSS2 and/or TMPRSS4 protein.

Illustrative gRNA sequences complementary to a sequence of a gene encoding a TMPRSS2 protein or a gene encoding a TMPRSS4 protein are provided in SEQ ID NOS: 1-436. Although the sequences are listed with DNA nucleotides, a person of ordinary skill would understand that the sequences are in fact RNA sequences and would be readily able to convert the DNA sequences into RNA sequences. In some embodiments, the gRNA sequences target regions early in the TMPRSS2 or TMPRSS4 coding sequence or in coding regions containing protease active site residues.

The gRNA can comprise a nucleotide sequence comprising one or more of SEQ ID NOs: 1-436. The gRNA can have a length of 100 nucleotides or fewer, 90 nucleotides or fewer, 80 nucleotides or fewer, 70 nucleotides or fewer, 60 nucleotides or fewer, 50 nucleotides or fewer, 40 nucleotides or fewer, 30 nucleotides or fewer, or 20 nucleotides or fewer. For example, the gRNA can have a length of 20 nucleotides.

In certain embodiments, guide RNAs within TMPRSS2 and TMPRSS4 are selected for their ability to generate in-frame stop codons when paired.

### DNA-Binding Polypeptides

In some embodiments, site-specific integration may be accomplished by utilizing factors that are capable of recognizing and binding to particular nucleotide sequences, for example, in the genome of a host organism. For instance, many proteins comprise polypeptide domains that are capable of recognizing and binding to DNA in a site-specific manner. A DNA sequence that is recognized by a DNA-binding polypeptide may be referred to as a “target” sequence. Polypeptide domains that are capable of recognizing and binding to DNA in a site-specific manner generally fold correctly and function independently to bind DNA in a site-specific manner, even when expressed in a polypeptide other than the protein from which the domain was originally isolated. Similarly, target sequences for recognition and binding by DNA-binding polypeptides are generally able to be recognized and bound by such polypeptides, even when present in large DNA structures (e.g., a chromosome), particularly when the site where the target sequence is located is one known to be accessible to soluble cellular proteins (e.g., a gene).

While DNA-binding polypeptides identified from proteins that exist in nature typically bind to a discrete nucleotide sequence or motif (e.g., a consensus recognition sequence), methods exist and are known in the art for modifying many such DNA-binding polypeptides to recognize a different nucleotide sequence or motif. DNA-binding polypeptides include, for example and without limitation: zinc finger DNA-binding domains; leucine zippers; UPA DNA-binding domains; GAL4; TAL; LexA; a Tet repressor; LacR; and a steroid hormone receptor.

In some embodiments, a DNA-binding polypeptide is a zinc finger. Individual zinc finger motifs can be designed to target and bind specifically to any of a large range of DNA sites. Canonical Cys₂His₂ (as well as non-canonical Cys₃His) zinc finger polypeptides bind DNA by inserting an α-helix into the major groove of the target DNA double helix. Recognition of DNA by a zinc finger is modular; each finger contacts primarily three consecutive base pairs in the target, and a few key residues in the polypeptide mediate recognition. By including multiple zinc finger DNA-binding domains in a targeting endonuclease, the DNA-binding specificity of the targeting endonuclease may be further increased (and hence the specificity of any gene regulatory effects conferred thereby may also be increased). See, e.g., Urnov, F. D., et al., Nature, 2005, 435, 646-651. Thus, one or more zinc finger DNA-binding polypeptides may be engineered and utilized such that a targeting endonuclease introduced into a host cell interacts with a DNA sequence that is unique within the genome of the host cell.

Preferably, the zinc finger protein is non-naturally occurring in that it is engineered to bind to a target site of choice. See, for example, Beerli, R. R., et al., Nature Biotechnol., 2002, 20, 135-141: Pabo, C. O., et al., Ann. Rev. Biochem., 2001, 70, 313-340; Isalan, M., et al., Nature Biotechnol., 2001, 19, 656-660; Segal, D. J., et al., Curr. Opin. Biotechnol., 2001, 12, 632-637; Choo, Y., et al., 2000, Curr. Opin. Struct. Biol., 10, 411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered zinc finger binding domain can have a novel binding specificity, compared to a naturally occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

Selection of target sites: ZFPs and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; 6,200,759; and published PCT applications WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536 and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

In some examples, a DNA-binding polypeptide is a DNA-binding domain from GAL4. GAL4 is a modular transactivator in Saccharomyces cerevisiae, but it also operates as a transactivator in many other organisms. See, e.g., Sadowski, I., et al., Nature, 1988, 335, 563-564. In this regulatory system, the expression of genes encoding enzymes of the galactose metabolic pathway in S. cerevisiae is stringently regulated by the available carbon source. Johnston, M., Microbiol. Rev., 1987, 51, 458-476. Transcriptional control of these metabolic enzymes is mediated by the interaction between the positive regulatory protein, GAL4, and a 17 bp symmetrical DNA sequence to which GAL4 specifically binds (the UAS).

Native GAL4 consists of 881 amino acid residues, with a molecular weight of 99 kDa. GAL4 comprises functionally autonomous domains, the combined activities of which account for activity of GAL4 in vivo (Ma, J. and Ptashne, M., Cell, 1987, 48, 847-853; Brent, R. and Ptashne, M., Cell, 1985 43, 729-736). The N-terminal 65 amino acids of GAL4 comprise the GAL4 DNA-binding domain (Keegan, L., et al., Science, 1986, 231, 699-704: Johnston, M., Nature, 1987, 328, 353-355). Sequence-specific binding requires the presence of a divalent cation coordinated by 6 Cys residues present in the DNA binding domain. The coordinated cation-containing domain interacts with and recognizes a conserved CCG triplet at each end of the 17 bp UAS via direct contacts with the major groove of the DNA helix (Marmorstein, M., et al., Nature, 1992, 356, 408-414). The DNA-binding function of the protein positions C-terminal transcriptional activating domains in the vicinity of the promoter, such that the activating domains can direct transcription.

Additional DNA-binding polypeptides that may be utilized in certain embodiments include, for example and without limitation, a binding sequence from a AVRBS3-inducible gene: a consensus binding sequence from a AVRBS3-inducible gene or synthetic binding sequence engineered therefrom (e.g., UPA DNA-binding domain); TAL; LexA (see, e.g., Brent, R. and Ptashne, M., Cell, 1985 43, 729-736); LacR (see, e.g., Labow, M. A., et al., Mol. Cell. Biol., 1990, 10, 3343-3356; Baim, S. B., et al., Proc. Natl. Acad. Sci. USA 1991, 88, 5072-5076); a steroid hormone receptor (Elliston, J. F., et al., J. Biol. Chem. 1990, 265, 11517-11521); the Tet repressor (U.S. Pat. No. 6,271,341) and a mutated Tet repressor that binds to a tet operator sequence in the presence, but not the absence, of tetracycline (Tc); the DNA-binding domain of NF-κB; and components of the regulatory system described in Wang, Y., et al., Proc. Natl. Acad. Sci. USA, 1994, 91, 8180-8184, which utilizes a fusion of GAL4, a hormone receptor, and VP16.

In certain embodiments, the DNA-binding domain of one or more of the nucleases used in the methods and compositions described herein comprises a naturally occurring or engineered (non-naturally occurring) TAL effector DNA binding domain. See, e.g., U.S. Patent Publication No. 20110301073, incorporated by reference in its entirety herein.

In other embodiments, the nuclease comprises a CRISPR system (e.g., CRISPR/Cas9 system). The CRISPR (clustered regularly interspaced short palindromic repeats) locus, which encodes RNA components of the system, and the Cas (CRISPR-associated) locus, which encodes proteins (Jansen, R., et al., Mol. Microbiol., 2002, 43, 1565-1575: Makarova, K. S., et al., Nucleic Acids Res. 2002, 30:482-496; Makarova, K. S., et al., Biol. Direct, 2006, 1, 7: Haft, D. H., et al., PLOS Comput. Biol., 2005, 1, e60) make up the gene sequences of the CRISPR/Cas nuclease system. CRISPR loci in microbial hosts contain a combination of Cas genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNAs, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the foreign nucleic acid. Thus, in the bacterial cell, several Cas proteins are involved with the natural function of the CRISPR/Cas system and serve roles in functions such as insertion of the foreign DNA etc.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

Cas9 protein comprises a RuvC nuclease domain and an HNH (H—N—H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and Ill, where domain I is located near the N-terminus of Cas9 and subdomains II and Ill are located in the middle of the protein, flanking the HNH domain (Hsu, P. D., et al., Cell, 2014, 157, 1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA that combines a crRNA and a tracrRNA into a single molecule. The amino acid sequence of a Cas9 protein described herein, as well as certain other Cas proteins herein, may be derived from a Streptococcus (e.g., S. pyogenes, S. pneumoniae, S. thermophilus, S. agalactiae, S. parasanguinis, S. oralis, S. salivarius, S. macacae, S. dysgalactiae, S. anginosus, S. constellatus, S. pseudoporcinus, S. mutans), Listeria (e.g., L. innocua), Spiroplasma (e.g., S. apis, S. syrphidicola), Peptostreptococcaceae, Atopobium, Porphyromonas (e.g., P. catoniae), Prevotella (e.g., P. intermedia), Veillonella, Treponema (e.g., T. socranskii, T. denticola), Capnocytophaga, Finegoldia (e.g., F. magna), Coriobacteriaceae (e.g., C. bacterium), Olsenella (e.g., O. profusa), Haemophilus (e.g., H. sputorum, H. pittmaniae), Pasteurella (e.g., P. bettyae), Olivibacter (e.g., O. sitiensis), Epilithonimonas (e.g., E. tenax), Mesonia (e.g., M. mobilis), Lactobacillus (e.g., L. plantarum), Bacillus (e.g., B. cereus), Aquimarina (e.g., A. muelleri), Chryseobacterium (e.g., C. palustre), Bacteroides (e.g., B. graminisolvens), Neisseria (e.g., N. meningitidis), Francisella (e.g., F. novicida), or Flavobacterium (e.g., F. frigidarium, F. soli) species, for example. As another example, a Cas9 protein can be any of the Cas9 proteins disclosed in Chylinski et al. (Chylinski, K., et al., RNA Biology, 2013, 10, 726-737 and U.S. patent application 62/162,377, filed May 15, 2015), which are incorporated herein by reference.

Accordingly, the sequence of a Cas9 protein herein can comprise, for example, any of the Cas9 amino acid sequences disclosed in GenBank Accession Nos. G3ECR1 (S. thermophilus), WP_026709422, WP_027202655, WP_027318179, WP_027347504, WP_027376815, WP_027414302, WP_027821588, WP_027886314, WP_027963583, WP_028123848, WP_028298935, Q03J16 (S. thermophilus), EGP66723, EGS38969, EGV05092, EH165578 (S. pseudoporcinus), EIC75614 (S. oralis), EID22027 (S. constellatus), EIJ69711, EJP22331 (S. oralis), EJP26004 (S. anginosus), EJP30321, EPZ44001 (S. pyogenes), EPZ46028 (S. pyogenes), EQL78043 (S. pyogenes), EQL78548 (S. pyogenes), ERL10511, ERL12345, ERL19088 (S. pyogenes), ESA57807 (S. pyogenes), ESA59254 (S. pyogenes), ESU85303 (S. pyogenes), ETS96804, UC75522, EGR87316 (S. dysgalactiae), EGS33732, EGV01468 (S. oralis), EHJ52063 (S. macacae), EID26207 (S. oralis), EID33364, EIG27013 (S. parasanguinis), EJF37476, EJO19166 (Streptococcus sp. BS35b), EJU16049, EJU32481, YP_006298249, ERF61304, ERK04546, ETJ95568 (S. agalactiae), TS89875, ETS90967 (Streptococcus sp. SR4), ETS92439, EUB27844, (Streptococcus sp. BS21), AFJ08616, EUC82735 (Streptococcus sp. CM6), EWC92088, EWC94390, EJP25691, YP_008027038, YP_008868573, AGM26527, AHK22391, AHB36273, Q927P4, G3ECR1, or Q99ZW2 (S. pyogenes), which are incorporated by reference. A variant of any of these Cas9 protein sequences may be used, but should have specific binding activity, and optionally endonucleolytic activity, toward DNA when associated with an RNA component herein. Such a variant may comprise an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of the reference Cas9. Alternatively, a Cas9 protein may comprise an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to any of the foregoing amino acid sequences, for example. Such a variant Cas9 protein should have specific binding activity, and optionally cleavage or nicking activity, toward DNA when associated with an RNA component herein. A Cas protein herein such as a Cas9 can comprise a heterologous nuclear localization sequence (NLS). A heterologous NLS amino acid sequence herein may be of sufficient strength to drive accumulation of a Cas protein in a detectable amount in the nucleus of a yeast cell herein, for example. An NLS may comprise one (monopartite) or more (e.g., bipartite) short sequences (e.g., 2 to 20 residues) of basic, positively charged residues (e.g., lysine and/or arginine), and can be located anywhere in a Cas amino acid sequence but such that it is exposed on the protein surface. An NLS may be operably linked to the N-terminus or C-terminus of a Cas protein herein, for example. Two or more NLS sequences can be linked to a Cas protein, for example, such as on both the N- and C-termini of a Cas protein. Non-limiting examples of suitable NLS sequences herein include those disclosed in U.S. Pat. No. 7,309,576, which is incorporated herein by reference.

The Cas endonuclease can comprise a modified form of the Cas9 polypeptide. The modified form of the Cas9 polypeptide can include an amino acid change (e.g., deletion, insertion, or substitution) that reduces the naturally occurring nuclease activity of the Cas9 protein. For example, in some instances, the modified form of the Cas9 protein has less than 50%, less than 40%, less than 30%, less than 20%, less than 10%, less than 5%, or less than 1% of the nuclease activity of the corresponding wild-type Cas9 polypeptide (US patent application US20140068797 A1, published on Mar. 6, 2014). In some cases, the modified form of the Cas9 polypeptide has no substantial nuclease activity and is referred to as catalytically “inactivated Cas9” or “deactivated cas9 (dCas9).” Catalytically inactivated Cas9 variants include Cas9 variants that contain mutations in the HNH and RuvC nuclease domains. These catalytically inactivated Cas9 variants are capable of interacting with sgRNA and binding to the target site in vivo but cannot cleave either strand of the target DNA.

A catalytically inactive Cas9 can be fused to a heterologous sequence (US patent application US20140068797 A1, published on Mar. 6, 2014). Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A catalytically inactive Cas9 can also be fused to a FokI nuclease to generate double-strand breaks (Guilinger, J. P., et al., Nature Biotechnology, 2014, 32, 577-582).

In particular embodiments, a DNA-binding polypeptide specifically recognizes and binds to a target nucleotide sequence comprised within a genomic nucleic acid of a host organism. Any number of discrete instances of the target nucleotide sequence may be found in the host genome in some examples. The target nucleotide sequence may be rare within the genome of the organism (e.g., fewer than about 10, about 9, about 8, about 7, about 6, about 5, about 4, about 3, about 2, or about 1 copy(ies) of the target sequence may exist in the genome). For example, the target nucleotide sequence may be located at a unique site within the genome of the organism. Target nucleotide sequences may be, for example and without limitation, randomly dispersed throughout the genome with respect to one another: located in different linkage groups in the genome: located in the same linkage group; located on different chromosomes; located on the same chromosome; located in the genome at sites that are expressed under similar conditions in the organism (e.g., under the control of the same, or substantially functionally identical, regulatory factors); and located closely to one another in the genome (e.g., target sequences may be comprised within nucleic acids integrated as concatemers at genomic loci).

### Targeting Endonucleases

In particular embodiments, a DNA-binding polypeptide that specifically recognizes and binds to a target nucleotide sequence may be comprised within a chimeric polypeptide, so as to confer specific binding to the target sequence upon the chimeric polypeptide. In examples, such a chimeric polypeptide may comprise, for example and without limitation, nuclease, recombinase, and/or ligase polypeptides, as these polypeptides are described above. Chimeric polypeptides comprising a DNA-binding polypeptide and a nuclease, recombinase, and/or ligase polypeptide may also comprise other functional polypeptide motifs and/or domains, such as for example and without limitation: a spacer sequence positioned between the functional polypeptides in the chimeric protein: a leader peptide: a peptide that targets the fusion protein to an organelle (e.g., the nucleus): polypeptides that are cleaved by a cellular enzyme: peptide tags (e.g., Myc, His, etc.); and other amino acid sequences that do not interfere with the function of the chimeric polypeptide.

Functional polypeptides (e.g., DNA-binding polypeptides and nuclease polypeptides) in a chimeric polypeptide may be operatively linked. In some embodiments, functional polypeptides of a chimeric polypeptide may be operatively linked by their expression from a single polynucleotide encoding at least the functional polypeptides ligated to each other in-frame, so as to create a chimeric gene encoding a chimeric protein. In alternative embodiments, the functional polypeptides of a chimeric polypeptide may be operatively linked by other means, such as by cross-linkage of independently expressed polypeptides.

In some embodiments, a DNA-binding polypeptide, or guide RNA that specifically recognizes and binds to a target nucleotide sequence may be comprised within a natural isolated protein (or mutant thereof), wherein the natural isolated protein or mutant thereof also comprises a nuclease polypeptide (and may also comprise a recombinase and/or ligase polypeptide). Examples of such isolated proteins include TALENs, recombinases (e.g., Cre, Hin, Tre, and FLP recombinase), CRISPR systems (e.g., CRISPR/Cas9 systems), and meganucleases.

As used herein, the term “targeting endonuclease” refers to natural or engineered isolated proteins and mutants thereof that comprise a DNA-binding polypeptide or guide RNA and a nuclease polypeptide, as well as to chimeric polypeptides comprising a DNA-binding polypeptide or guide RNA and a nuclease. Any targeting endonuclease comprising a DNA-binding polypeptide or guide RNA that specifically recognizes and binds to a target nucleotide sequence comprised within a TMPRSS2 or TMPRSS4 locus (e.g., either because the target sequence is comprised within the native sequence at the locus, or because the target sequence has been introduced into the locus, for example, by recombination) may be utilized in certain embodiments.

Some examples of chimeric polypeptides that may be useful in particular embodiments of the invention include, without limitation, combinations of the following polypeptides: zinc finger DNA-binding polypeptides; a FokI nuclease polypeptide; TALE domains; leucine zippers; transcription factor DNA-binding motifs; and DNA recognition and/or cleavage domains isolated from, for example and without limitation, a TALEN, a recombinase (e.g., Cre, Hin, RecA, Tre, and FLP recombinases), a CRISPR system (e.g., CRISPR/Cas9 system), a meganuclease; and others known to those in the art. Particular examples include a chimeric protein comprising a site-specific DNA binding polypeptide and a nuclease polypeptide. Chimeric polypeptides may be engineered by methods known to those of skill in the art to alter the recognition sequence of a DNA-binding polypeptide comprised within the chimeric polypeptide, so as to target the chimeric polypeptide to a particular nucleotide sequence of interest.

In certain embodiments, the chimeric polypeptide comprises a DNA-binding domain (e.g., zinc finger, TAL-effector domain, etc.) and a nuclease (cleavage) domain. The cleavage domain may be heterologous to the DNA-binding domain, for example a zinc finger DNA-binding domain and a cleavage domain from a nuclease or a TALEN DNA-binding domain and a cleavage domain, or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort, M. and Roberts, J., Nucleic Acids Res., 1997, 25, 3379-3388. Additional enzymes which cleave DNA are known (e.g., 51 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However, any integral number of nucleotides, or nucleotide pairs, can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding, for example, such that one or more exogenous sequences (donors/transgenes) are integrated at or near the binding (target) sites. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li, L., et al., Proc. Natl. Acad. Sci. USA, 1992, 89, 4275-4279; Li, L., et al., Proc. Natl. Acad. Sci. USA, 1993, 90, 2764-2768; Kim, Y-G., et al., Proc. Natl. Acad. Sci. USA, 1994, 91, 883-887; Kim, Y-G., et al., J. Biol. Chem., 1994, 269, 31978-31982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer (Bitinaite, J., et al., Proc. Natl. Acad. Sci. USA, 1998, 95, 10570-10575). Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a DNA binding domain and two Fok I cleavage half-domains can also be used.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in U.S. Patent Publication No. 20070134796, incorporated herein in its entirety. Additional restriction enzymes also contain separable binding and cleavage domains, and these are contemplated by the present disclosure. See, for example, Roberts, R. J., et al., Nucleic Acids Res., 2003, 31, 418-420.

In certain embodiments, the cleavage domain comprises one or more engineered cleavage half-domain (also referred to as dimerization domain mutants) that minimize or prevent homodimerization, as described, for example, in U.S. Patent Publication Nos. 20050064474; 20060188987 and 20080131962, the disclosures of all of which are incorporated by reference in their entireties herein.

Alternatively, nucleases may be assembled in vivo at the nucleic acid target site using so-called “split-enzyme” technology (see e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

### Zinc Finger Nucleases

In specific embodiments, a chimeric polypeptide is a custom-designed zinc finger nuclease (ZFN) that may be designed to deliver a targeted site-specific double-strand DNA break into which an exogenous nucleic acid, or donor DNA, may be integrated (See US Patent publication 20100257638, incorporated by reference herein). ZFNs are chimeric polypeptides containing a non-specific cleavage domain from a restriction endonuclease (for example, FokI) and a zinc finger DNA-binding domain polypeptide. See, e.g., Huang, B., et al., J. Protein Chem., 1996, 15, 481-489; Kim, J-S., et al. Proc. Natl. Acad. Sci. USA, 1997, 94, 3616-3620; Kim, Y-G., et al., Proc. Natl. Acad. Sci. USA, 1996, 93, 1156-1160; Kim, Y-G., et al., Proc Natl. Acad. Sci. USA, 1994, 91, 883-887; Kim, Y-G., et al., Proc. Natl. Acad. Sci. USA, 1997, 94, 12875-12879; Kim, Y-G., et al., Gene, 1997, 203, 43-49; Kim, Y-G., et al., Biol. Chem., 1998, 379, 489-495; Nahon, E. and Raveh, D., Nucleic Acids Res., 1998, 26, 1233-1239; Smith, J., et al., Nucleic Acids Res., 1999, 27, 674-681. In some embodiments, the ZFNs comprise non-canonical zinc finger DNA binding domains (see US Patent publication 20080182332, incorporated by reference herein). The FokI restriction endonuclease must dimerize via the nuclease domain in order to cleave DNA and introduce a double-strand break. Consequently, ZFNs containing a nuclease domain from such an endonuclease also require dimerization of the nuclease domain in order to cleave target DNA (Mani, M. et al., Biochem. Biophys. Res. Commun., 2005, 334, 1191-1197; Smith, J., et al., Nucleic Acids Res. 2000, 28, 3361-3369). Dimerization of the ZFN can be facilitated by two adjacent, oppositely oriented DNA-binding sites (Id.).

In particular examples, a method for the site-specific integration of an exogenous nucleic acid into the TMPRSS2 or TMPRSS4 locus of a host comprises introducing into a cell of the host a ZFN, wherein the ZFN recognizes and binds to a target nucleotide sequence, wherein the target nucleotide sequence is comprised within the TMPRSS2 or TMPRSS4 locus of the host. In certain examples, the target nucleotide sequence is not comprised within the genome of the host at any other position than the TMPRSS2 or TMPRSS4 locus. For example, a DNA-binding polypeptide of the ZFN may be engineered to recognize and bind to a target nucleotide sequence identified within the TMPRSS2 or TMPRSS4 locus (e.g., by sequencing the TMPRSS2 or TMPRSS4 locus). A method for the site-specific integration of an exogenous nucleic acid into the TMPRSS2 or TMPRSS4 locus of a host that comprises introducing into a cell of the host a ZFN may also comprise introducing into the cell an exogenous nucleic acid, wherein recombination of the exogenous nucleic acid into a nucleic acid of the host comprising the TMPRSS2 or TMPRSS4 locus is facilitated by site-specific recognition and binding of the ZFN to the target sequence (and subsequent cleavage of the nucleic acid comprising the TMPRSS2 or TMPRSS4 locus).

### Optional Exogenous Nucleic Acids for Integration at a TMPRSS2 or TMPRSS4 Locus

Embodiments of the invention may include one or more nucleic acids selected from the group consisting of: an exogenous nucleic acid for site-specific integration in a TMPRSS2 or TMPRSS4 locus, for example and without limitation, an ORF: a nucleic acid comprising a nucleotide sequence encoding a targeting endonuclease; and a vector comprising at least one of either or both of the foregoing. Thus, particular nucleic acids for use in some embodiments include nucleotide sequences encoding a polypeptide, structural nucleotide sequences, and/or DNA-binding polypeptide recognition and binding sites.

### Optional Exogenous Nucleic Acid Molecules for Site-Specific Integration

As noted above, insertion of an exogenous sequence (also called a “donor sequence” or “donor”) is provided, for example for expression of a polypeptide, correction of a mutant gene, or for increased or decreased expression of a wild-type gene. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded or double-stranded and can be introduced into a cell in linear or circular form. See e.g., U.S. Patent Publication Nos. 20100047805, 20110281361, 20110207221 and U.S. application Ser. No. 13/889,162. If introduced in linear form, the ends of the donor sequence can be protected (e.g. from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang, X B. and Wilson, J. H., Proc. Natl. Acad. Sci. USA, 1987, 84, 4959-4963: Nehls, M., et al., Science, 1996, 272, 886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally integrated so that its expression is driven by the endogenous promoter at the integration site, namely the promoter that drives expression of the endogenous gene into which the donor is integrated (e.g., TMPRSS2 and/or TMPRSS4). However, it will be apparent that the donor may comprise a promoter and/or enhancer, for example a constitutive promoter or an inducible or tissue-specific promoter.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

Exogenous nucleic acids that may be integrated in a site-specific manner into a TMPRSS2 and/or TMPRSS4 locus, so as to modify the TMPRSS2 and/or TMPRSS4 locus, in embodiments include, for example and without limitation, nucleic acids comprising a nucleotide sequence encoding a polypeptide of interest; nucleic acids comprising an agronomic gene; nucleic acids comprising a nucleotide sequence encoding an RNAi molecule; or nucleic acids that disrupt the TMPRSS2 and/or TMPRSS4 gene.

In some embodiments, an exogenous nucleic acid is integrated at a TMPRSS2 and/or TMPRSS4 locus, so as to modify the TMPRSS2 and/or TMPRSS4 locus, wherein the nucleic acid comprises a nucleotide sequence encoding a polypeptide of interest, such that the nucleotide sequence is expressed in the host from the TMPRSS2 and/or TMPRSS4 locus. In some examples, the polypeptide of interest (e.g., a foreign protein) is expressed from a nucleotide sequence encoding the polypeptide of interest in commercial quantities. In such examples, the polypeptide of interest may be extracted from the host cell, tissue, or biomass.

### Nucleic Acid Molecules Comprising a Nucleotide Sequence Encoding a Targeting Endonuclease

In some embodiments, a nucleotide sequence encoding a targeting endonuclease may be engineered by manipulation (e.g., ligation) of native nucleotide sequences encoding polypeptides comprised within the targeting endonuclease. For example, the nucleotide sequence of a gene encoding a protein comprising a DNA-binding polypeptide may be inspected to identify the nucleotide sequence of the gene that corresponds to the DNA-binding polypeptide, and that nucleotide sequence may be used as an element of a nucleotide sequence encoding a targeting endonuclease comprising the DNA-binding polypeptide. Alternatively, the amino acid sequence of a targeting endonuclease may be used to deduce a nucleotide sequence encoding the targeting endonuclease, for example, according to the degeneracy of the genetic code.

In exemplary nucleic acid molecules comprising a nucleotide sequence encoding a targeting endonuclease, the last codon of a first polynucleotide sequence encoding a nuclease polypeptide, and the first codon of a second polynucleotide sequence encoding a DNA-binding polypeptide, may be separated by any number of nucleotide triplets, e.g., without coding for an intron or a “STOP.” Likewise, the last codon of a nucleotide sequence encoding a first polynucleotide sequence encoding a DNA-binding polypeptide, and the first codon of a second polynucleotide sequence encoding a nuclease polypeptide, may be separated by any number of nucleotide triplets. In these and further embodiments, the last codon of the last (i.e., most 3′ in the nucleic acid sequence) of a first polynucleotide sequence encoding a nuclease polypeptide, and a second polynucleotide sequence encoding a DNA-binding polypeptide, may be fused in phase-register with the first codon of a further polynucleotide coding sequence directly contiguous thereto, or separated therefrom by no more than a short peptide sequence, such as that encoded by a synthetic nucleotide linker (e.g., a nucleotide linker that may have been used to achieve the fusion). Examples of such further polynucleotide sequences include, for example and without limitation, tags, targeting peptides, and enzymatic cleavage sites. Likewise, the first codon of the most 5′ (in the nucleic acid sequence) of the first and second polynucleotide sequences may be fused in phase-register with the last codon of a further polynucleotide coding sequence directly contiguous thereto or separated therefrom by no more than a short peptide sequence.

A sequence separating polynucleotide sequences encoding functional polypeptides in a targeting endonuclease (e.g., a DNA-binding polypeptide and a nuclease polypeptide) may, for example, consist of any sequence, such that the amino acid sequence encoded is not likely to significantly alter the translation of the targeting endonuclease. Due to the autonomous nature of known nuclease polypeptides and known DNA-binding polypeptides, intervening sequences will not in examples interfere with the respective functions of these structures.

### Other Knockout Methods

Various other techniques known in the art can be used to inactivate genes to make knock-out animals and/or to introduce nucleic acid constructs into animals to produce founder animals, in which the knockout or nucleic acid construct is integrated into the genome. Such techniques include, without limitation, pronuclear microinjection (U.S. Pat. No. 4,873,191), retrovirus mediated gene transfer into germ lines (Van der Putten H., et al., Proc. Natl. Acad. Sci. USA, 1985, 82, 6148-1652), gene targeting into embryonic stem cells (Thompson, S., et al., Cell, 1989, 56, 313-321), electroporation of embryos (Lo, W., Mol. Cell. Biol., 1983, 3, 1803-1814), sperm-mediated gene transfer (Lavitrano, M., et al., Proc. Natl. Acad. Sci. USA, 2002 99, 14230-14235: Lavitrano, M., et al., Reprod. Fert. Develop., 2006, 18, 19-23), and in vitro transformation of somatic cells, such as cumulus or mammary cells, or adult, fetal, or embryonic stem cells, followed by nuclear transplantation (Wilmut, I, et al., Nature, 1997, 385, 810-813; and Wakayama, T., et al., Nature, 1998, 394, 369-374). Pronuclear microinjection, sperm mediated gene transfer, and somatic cell nuclear transfer are particularly useful techniques. An animal that is genomically edited is an animal wherein all of its cells have the genetic edit, including its germ line cells. When methods are used that produce an animal that is mosaic in its genetic edit, the animals may be bred and progeny that are genomically edited (that is have inherited the gene edit and carry it in all their cells) may be selected. Animals that comprise an edited gene that confers reduced susceptibility to infection by an influenza virus can be homozygous or heterozygous for the edit, depending on the specific approach that is used. Animals that are edited for reduced susceptibility to infection by an influenza virus may also have multiple edits, such as both TMPRSS2 and TMPRSS4, or these edits may be stacked with edits that confer other desirable traits. If a particular gene is inactivated by a knock-out modification, homozygosity would normally be required. If a particular gene is inactivated by an RNA interference or dominant negative strategy, then heterozygosity is often adequate.

Typically, in embryo/zygote microinjection, a nucleic acid construct or mRNA is introduced into a fertilized egg: 1 or 2 cell fertilized eggs are used as the pronuclei containing the genetic material from the sperm head and the egg are visible within the protoplasm. Pronuclear staged fertilized eggs can be obtained in vitro or in vivo (i.e., surgically recovered from the oviduct of donor animals). An exemplary protocol for producing in vitro fertilized egg follows. Swine ovaries can be collected at an abattoir, and maintained at 22-28° C. during transport. Ovaries can be washed and isolated for follicular aspiration, and follicles ranging from 4-8 mm can be aspirated into 50 ml conical centrifuge tubes using 18-gauge needles and under vacuum to suck the oocyte from the follicle on the ovary. Follicular fluid and aspirated oocytes can be rinsed through pre-filters with commercial TL-HEPES (Minitube, Verona, WI). Oocytes surrounded by a compact cumulus mass can be selected and placed into TCM-199 oocyte maturation medium (Minitube, Verona, WI) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO₂. Subsequently, the oocytes can be moved to fresh TCM-199 maturation medium, which will not contain cAMP, PMSG or hCG and then the oocytes can be incubated for an additional 22 hours. Matured oocytes can be stripped of their cumulus cells by vortexing in 0.1% hyaluronidase for 1 minute.

For swine, mature oocytes can be fertilized in 500 μl Minitube PORCPRO IVF medium system (Minitube, Verona, Wis.) in Minitube 5-well fertilization dishes. In preparation for in vitro fertilization (IVF), freshly-collected or frozen boar semen can be washed and resuspended in PORCPRO IVF medium to 4×10⁵ sperm. Sperm concentrations can be analyzed by computer assisted semen analysis (SPERMVISION, Minitube, Verona, WI). Final in vitro insemination can be performed in a 10 μl volume at a final concentration of approximately 40 motile sperm/oocyte, depending on the boar. The oocytes can be incubated at 38.7° C. in 5.0% CO₂ atmosphere for 6 hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs or mRNA can be injected into one of the pronuclei or into the cytoplasm. Then the injected eggs can be transferred to a recipient female (e.g., into the oviducts of a recipient female) and allowed to develop in the recipient female to produce the genetically edited animals. In particular, in vitro fertilized embryos can be centrifuged at 15,000×g for 5 minutes to sediment lipids allowing visualization of the pronucleus. The embryos can be injected with using an EPPENDORF® FEMTOJET® injector (EPPENDORF®, Hamburg, Germany) and can be cultured until blastocyst formation. Rates of embryo cleavage and blastocyst formation and quality can be recorded.

Embryos can be surgically transferred into uteri of asynchronous recipients. Typically, 20-200 embryos can be deposited into the ampulla-isthmus junction of the oviduct using a catheter. After surgery, real-time ultrasound examination of pregnancy can be performed.

In somatic cell nuclear transfer (SCNT), a gene edited cell (e.g., a gene edited pig cell) such as an embryonic blastomere, fetal fibroblast, adult ear fibroblast, or granulosa cell that has a gene edit of the present teachings can be introduced into an enucleated oocyte to establish a combined cell. (Such somatic cells can be edited as described, for example but without limitation, as described in Example 1.) Oocytes can be enucleated by partial zona dissection near the polar body and then pressing out cytoplasm at the dissection area. Typically, an injection pipette with a sharp beveled tip is used to inject the genetically edited cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli, J. B., et al., Science, 1998, 280, 1256-1258 and U.S. Pat. No. 6,548,741. For pigs, recipient females can be checked for pregnancy approximately 20-21 days after transfer of the embryos.

Standard breeding techniques can be used to create animals that are homozygous for the desired edit from the initial heterozygous founder animals. Homozygosity may not be required, however. Gene edited animals described herein can be bred with other animals of interest. Standard breeding techniques may also be used to mate a TMPRSS2 edited pig with a TMPRSS4 edited pig, thus creating a pig comprising TMPRSS2 and TMPRSS4 edits. Edits disclosed herein may also be combined with other traits such as a health trait, a growth trait, or a production trait. Edited CD163 genes that confer resistance to PRRS virus are of particular interest.

In some embodiments, a nucleic acid of interest and a selectable marker can be provided on separate transposons and provided to either embryos or cells in unequal amount, where the amount of transposon containing the selectable marker far exceeds (5-10 fold excess) the transposon containing the nucleic acid of interest. Genetically edited cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, genetically modified animals can be produced that are not constrained to retain selectable markers in subsequent generations.

Once genetically modified animals have been generated, expression of a nucleic acid can be assessed using standard techniques. Initial screening can be accomplished by ILLUMINA® sequencing or Southern blot analysis to determine whether or not an edit has taken place.

Expression of a nucleic acid sequence encoding a polypeptide in the tissues of genetically modified animals can be assessed using techniques that include, for example, Northern blot analysis of tissue samples obtained from the animal, in situ hybridization analysis, Western analysis, immunoassays such as enzyme-linked immunosorbent assays, and reverse-transcriptase PCR (RT-PCR).

### Interfering RNAs

A variety of interfering RNA (RNAi) systems are known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double-stranded RNAse (dsRNase, e.g., Dicer) and ssRNase (e.g., Argonaut 2 or Ago2). RISC utilizes antisense strand as a guide to find a cleavable target. Both siRNAs and microRNAs (miRNAs) are known. A method of inactivating a gene in a genetically edited animal comprises inducing RNA interference against a target gene and/or nucleic acid such that expression of the target gene and/or nucleic acid is reduced. Endogenous microRNAs can be edited to target endogenous genes of the present teachings.

For example, the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. Constructs for siRNA can be produced as described, for example, in Fire, A., et al., Nature, 1998, 391, 806-811: Romano, N. and Masino, G., Mol. Microbiol., 1992, 6, 3343-3353: Cogoni, C., et al., EMBO J., 1996, 15, 3153-3161: Cogoni, C. and Masino, G., Nature, 1999, 399, 166-169: Misquitta, L. and Paterson, B. M., Proc. Natl. Acad. Sci. USA, 1999, 96, 1451-1456; and Kennerdell, K. and Carthew, C., Cell, 1998 95, 1017-1026. Constructs for shRNA can be produced as described by Mcintyre, G. J. and Fanning, G. C., BMC Biotechnology, 2006, 6, 1-8. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective.

Embodiments include an in vitro cell, an in vivo cell, and a gene edited animal such as a livestock animal that expresses an RNAi directed against a TMPRSS2 or TMPRSS4 gene. The RNAi may be, for instance, selected from the group consisting of siRNA, shRNA, dsRNA, and miRNA.

### Inducible Systems

An inducible system may be used to control expression of a TMPRSS2 or TMPRSS4 gene. Various inducible systems are known that allow spatiotemporal control of expression of a gene. Several have been proven to be functional in vivo in animal systems.

An example of an inducible system is the tetracycline (tet)-on promoter system, which can be used to regulate transcription of the nucleic acid. In this system, a mutated Tet repressor (TetR) is fused to the activation domain of herpes simplex virus VP 16 trans-activator protein to create a tetracycline-controlled transcriptional activator (TA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically edited animals. One animal line expresses the activator (tTA, rtTA, or Cre recombinase) under the control of a selected promoter. Another set of genetically modified animals express the acceptor, in which the expression of the gene of interest (or the gene to be modified) is under the control of the target sequence for the tTA/rtTA transactivators (or is flanked by loxP sequences). Mating the two animal lines provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of gene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed floxed DNA) is excised by Cre-mediated recombination. Control of Cre expression in a genetically modified animal, using either spatial control (with a tissue- or cell-specific promoter), or temporal control (with an inducible system), results in control of DNA excision between the two loxP sites. One application is for conditional gene inactivation (conditional knockout). Another approach is for protein over-expression, wherein a floxed stop codon is inserted between the promoter sequence and the DNA of interest. Gene edited animals do not express the modified gene until Cre is expressed, leading to excision of the floxed stop codon. This system has been applied to tissue-specific oncogenesis and controlled antigene receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

Embodiments include an in vitro cell, an in vivo cell, and a gene edited animal such as a porcine animal that comprises a TMPRSS2 and/or TMPRSS4 gene that is under control of an inducible system. The genetic modification of an animal may be genomic (included in all animal cells) or mosaic (included in only some cells within an animal). The inducible system may be, for instance, selected from the group consisting of Tet-On, Tet-Off, Cre-lox, and Hif1 alpha.

### Vectors and Nucleic Acids

A variety of nucleic acids may be introduced into cells for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, Summerton, J. and Weller, D., Antisense Nucleic Acid Drug Dev., 1997, 7, 187-195; and Hyrup, B and Nielsen, P. E., Bioorgan. Med. Chem., 1996, 4, 5-23. In addition, the deoxyphosphate backbone can be replaced with, for example, a phosphorothioate or phosphorodithioate backbone, a phosphoroamidite, or an alkyl phosphotriester backbone.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

Any type of promoter can be operably linked to a target nucleic acid sequence. Examples of promoters include, without limitation, tissue-specific promoters, constitutive promoters, inducible promoters, and promoters responsive or unresponsive to a particular stimulus. Suitable tissue-specific promoters can result in preferential expression of a nucleic acid transcript in beta cells and include, for example, the human insulin promoter. Other tissue-specific promoters can result in preferential expression in, for example, hepatocytes or heart tissue and can include the albumin or alpha-myosin heavy chain promoters, respectively. In other embodiments, a promoter that facilitates the expression of a nucleic acid molecule without significant tissue or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu, L., et al., Hum. Gene Ther., 2001, 12, 563-573; and Kiwaki, K., et al., Hum. Gene Ther., 1996, 7, 821-830.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphotransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban, P. C., et al., Proc. Natl. Acad. Sci., 1992, 89, 6861-6865, for a review of Cre/lox technology, and Brand, C. S. and Dymecki, S. M., Dev. Cell, 2004, 6, 7-28. A transposon containing a Cre- or Flp-activatable transgene interrupted by a selectable marker gene also can be used to obtain genetically modified animals with conditional expression of a transgene. For example, a promoter driving expression of the marker/transgene can be either ubiquitous or tissue-specific, which would result in the ubiquitous or tissue-specific expression of the marker in FO animals. Tissue-specific activation of the transgene can be accomplished, for example, by crossing an animal that ubiquitously expresses a marker-interrupted transgene to an animal expressing Cre or Flp in a tissue-specific manner, or by crossing an animal that expresses a marker-interrupted transgene in a tissue-specific manner to an animal that ubiquitously expresses Cre or Flp recombinase. Controlled expression of the transgene or controlled excision of the marker allows expression of the transgene.

In some embodiments, the exogenous nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

Nucleic acid constructs can be methylated using an SssI CpG methylase (New England Biolabs, Ipswich, Mass.). In general, the nucleic acid construct can be incubated with S-adenosylmethionine and SssI CpG-methylase in buffer at 37° C. Hypermethylation can be confirmed by incubating the construct with one unit of HinPII endonuclease for 1 hour at 37° C. and assaying by agarose gel electrophoresis.

Nucleic acid constructs can be introduced into embryonic, fetal, or adult animal cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. Publication No. 2005/0003542); Frog Prince (Miskey, C., et al., Nucleic Acids Res., 2003, 31, 6873-6881); Tol2 (Kawakami, K., Genome Biology, 2007, 8 (Suppl. 1), S7; Minos (Pavlopoulos et al., Genome Biology, 2007, 8 (Suppl. 1), S2); Hsmar1 (Miskey, C., et al., Mol. Cell. Biol., 2007, 27, 4589-4600); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Insulator elements also can be included in a nucleic acid construct to maintain expression of the exogenous nucleic acid and to inhibit the unwanted transcription of host genes. See, for example, U.S. Publication No. 2004/0203158. Typically, an insulator element flanks each side of the transcriptional unit and is internal to the inverted repeat of the transposon. Non-limiting examples of insulator elements include the matrix attachment region-(MAR) type insulator elements and border-type insulator elements. See, for example, U.S. Pat. Nos. 6,395,549, 5,731,178, 6,100,448, and 5,610,053, and U.S. Publication No. 2004/0203158.

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggy Bac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand).

### Founder Animals, Traits, and Reproduction

Founder animals may be produced by cloning and other methods described herein. The founders can be homozygous for a genetic modification, as in the case where a zygote or a primary cell undergoes a homozygous modification. Similarly, founders can also be made that are heterozygous. The founders may be genomically edited, meaning that all of the cells in their genome carry the gene edit. Founders can also be mosaic for an edit, as may happen, without limitation, when editing reagents are introduced into one of a plurality of cells in an embryo, typically at the blastocyst stage. Without being limited by theory, mosaicism may also occur because the half life of the RNPs extends beyond the first cell division of a zygote. Progeny of mosaic animals may be tested to identify progeny that carry the edited gene.

In livestock, many alleles are known to be linked to various traits such as production traits, type traits, workability traits, and other functional traits. Practitioners are accustomed to monitoring and quantifying these traits, e.g., Visscher, P. M., et al., Livestock Production Science, 1994, 40, 123-137, U.S. Pat. No. 7,709,206, US 2001/0016315, US 2011/0023140, and US 2005/0153317. An animal may include a trait chosen from a trait in the group consisting of a production trait, a type trait, a workability trait, a fertility trait, a mothering trait, and a disease resistance trait. Further traits include expression of a recombinant gene product. Animals with a desired trait or traits may be modified to increase their resistance to infection with an influenza virus or a coronavirus.

In addition to monitoring traits, artisans can look at the genetic background of the animal as a whole. Gene edits such as those of the present teachings may be made in elite genetic backgrounds. Elite PIC™ (Pig Improvement Company, Limited, Basingstoke, UK) lines 2, 3, 15, 19, 27, 62 and 65 are lines selected for superior commercial phenotypes.

Other potential porcine lines include lines can be PIC™ Line 15, PIC™ Line 17. PIC™ Line 27, PIC™ Line 65, PIC™ Line 14, PIC™ Line 62, PIC337, PIC800, PIC280, PIC327. PIC408, PIC™ 399, PIC410, PIC415, PIC359, PIC380, PIC837, PIC260, PIC265, PIC210, PIC™ Line 2, PIC™ Line 3, PIC™ Line 4, PIC™ Line 5, PIC™ Line 18, PIC™ Line 19, PIC™ Line 92, PIC95, PIC™ CAMBOROUGH® (Pig Improvement Company, Limited, Basingstoke, UK), PIC1070, PIC™ CAMBOROUGH® 40, PIC™ CAMBOROUGH® 22, PIC1050, PIC™ CAMBOROUGH® 29, PIC™ CAMBOROUGH® 48, or PIC™ CAMBOROUGH® x54.

In various aspects, PIC™ Line 65 is sold under the trade name PIC337. In various aspects, PIC™ line 62 is sold under the tradename PIC408. In various aspects, hybrid pigs made by crossing PIC™ lines 15 and 17 are sold under the tradenames PIC800 or PIC280. In various aspects, PIC™ Line 27 is sold under the tradename PIC327. In various aspects, hybrids created from crossing PIC™ Line 65 and PIC™ Line 62 are sold under the tradenames PIC399, PIC410, or PIC415. In various aspects, hybrids created from crossing PIC™ Line 65 and Pic Line 27 are sold under the tradename PIC359. In various aspects, hybrids prepared from crossing PIC™ Line 800 pigs (which is a hybrid of PIC™ Line 15 and PIC™ Line 17) to PIC™ Line 65 pigs are sold under the tradenames PIC380 or PIC837. In various aspects, PIC™ Line 14 is sold under the trade name PIC260. In various aspects, hybrids created from crossing PIC™ Line 14 and PIC™ Line 65 are sold under the tradename PIC265. In various aspects, hybrids created by crossing PIC™ Line 2 and PIC™ Line 3 are sold under the tradenames PIC210, PIC™ CAMBOROUGH® and PIC1050. In various aspects, hybrids of PIC™ Line 3 and PIC™ Line 92 are sold under the tradename PIC95. In various configurations, hybrids made from crossing PIC™ Line 19 and PIC™ Line 3 are sold under the tradename PIC1070. In various aspects, hybrids created by crossing PIC™ Line 18 and PIC™ Line 3 are sold under the tradename PIC™ CAMBOROUGH® 40. In various aspects, hybrids created from crossing PIC™ Line 19 and PIC1050 (which is itself a hybrid of PIC™ lines 2 and 3) are sold under the tradename PIC™ CAMBOROUGH® 22. In various aspects, hybrids created from crossing PIC™ Line 2 and PIC1070 (which is itself a hybrid of PIC™ lines 19 and 3) are sold under the tradename PIC™ CAMBOROUGH® 29. In various aspects, hybrids created from crossing PIC™ Line 18 and PIC1050 (which is itself a hybrid of PIC™ lines 2 and 3) are sold under the tradename PIC™ CAMBOROUGH® 48. In various aspects, hybrids created from crossing PIC™ Line 4 and PIC™ Line 5 are sold under the tradename PIC™ CAMBOROUGH® x54

### EXAMPLES

### Example 1: Use of gRNAs to Introduce Premature Stop Codons into the Coding Regions of TMPRSS2 and TMPRSS4 without the Use of HDR Templates

This example demonstrates the use of gRNAs, in combination with Cas protein activity, to generate double-stranded breaks in DNA, which upon host-cell mediated non-homologous end-joining, results in the introduction of a premature stop codon in the coding regions of TMPRSS2 and TMPRSS4. Upon protein translation of the resultant mRNA encoded by the new TMPRSS2 or TMPRSS4, premature stop codons in TMPRSS2 and TMPRSS4 mRNA would result in premature termination of protein translation and ultimately truncated and non-functional proteins, and/or initiate nonsense-mediated mRNA decay resulting in elimination of TMPRSS2 and TMPRSS4 mRNA transcripts. Premature stop codons can be introduced via the homology directed repair (HDR) pathway by inclusion of a single- or double-stranded DNA template in editing experiments. However, exogenously added DNA can integrate randomly in the genome. Therefore, it would be advantageous to identify single gRNAs or gRNA pairs that promote the formation of in-frame stop codons, without the introduction of non-wild type amino acids. To accomplish this, one or two gRNAs can be used to direct nuclease cut sites, which are then repaired by NHEJ to affect the desired edit.

Guides within TMPRSS2 and TMPRSS4 were tested computationally for their ability to generate in-frame stop codons when paired. Computational predictions were subsequently tested in porcine fetal fibroblasts (PFFs), as described below. The gRNAs listed in Table 3 below were generated by in vitro transcription and complexed with SpyCas9 in water, using 3.2 μg of Cas9 protein and 2.2 μg of gRNA in a total volume of 2.23 μl. Resulting ribonucleoprotein (RNP) complexes were then combined 1:1 in a total volume of 2.23 μl to generate gRNA pairs, as indicated in Table 3, and the mixed RNP complexes were then nucleofected into PFFs using a Lonza electroporator. In preparation for nucleofection, PFF cells were harvested using TrypLE Express™ (ThermoFisher, recombinant Trypsin): the culture medium was removed from cells, cells were washed once with Hank's Balanced Salt Solution (HBSS) or Dulbecco's Phosphate-Buffered Saline (DPBS), and incubated for 3-5 minutes at 38.5° C. in the presence of TrypLE. Cells were then harvested with complete medium. Cells were pelleted via centrifugation (300 g×5 minutes at room temperature), supernatant was discarded, and then the cells were resuspended in 10 ml PBS to obtain single cell suspensions to allow cell counting using trypan blue staining. After counting, cells were pelleted via centrifugation, the supernatant was discarded, and the cells were resuspended in nucleofection buffer P3 at a final concentration of 7.5×10⁶ cells/ml. 20 μl of the cell suspension was added to each well of a nucleofection cuvette containing the RNP mixture and then mixed gently to resuspend the cells. The RNP/cell mixture was then nucleofected with program CM138 (provided by the manufacturer). Following nucleofection, 80 μl of warm Embryonic Fibroblast Medium (EFM) (Dulbecco's Modified Eagle's Medium (DMEM) containing 2.77 mM glucose, 1.99 mM L-glutamine, and 0.5 mM sodium pyruvate, supplemented with 100 μM 2-Mercaptoethanol, 1X Eagle's minimum essential medium non-essential amino acids (MEM NEAA), 100 μg/mL Penicillin-Streptomycin, and 12% Fetal Bovine Serum) was added to each well. The suspensions were mixed gently by pipetting, and then 100 μl was transferred to a 12-well plate containing 900 μl of EFM pre-incubated at 38.5° C. The plate was then incubated at 38.5° C., 5% CO₂, 5% O₂, 90% N₂ for 48 hours. Forty-eight hours after nucleofection, genomic DNA was prepared from transfected and control PFF cells: 15 μl of QUICKEXTRACT™ DNA Extraction Solution (Lucigen, LGC Holdings, Teddington, UK) was added to pelleted cells, and the cells were then lysed by incubating for 10 minutes at 37° C. for 8 minutes at 65° C., and for 5 minutes at 95° C. Lysate was held at 4° C. until used for DNA sequencing.

To evaluate NHEJ repair outcomes at TMPRSS2 and TMPRSS4 target sites mediated by the guide RNA/Cas endonuclease system, a region of approximately 250 bp of genomic DNA surrounding the target site was amplified by PCR and then the PCR product was examined by amplicon deep sequencing for the presence and nature of repairs. After transfection in triplicate, PFF genomic DNA was extracted and the region surrounding the intended target site was PCR amplified with Q5 Polymerase (NEB) adding sequences necessary for amplicon-specific barcodes and ILLUMINA® sequencing using tailed primers through two rounds of PCR, respectively. The resulting PCR amplification products were deep sequenced on an ILLUMINA® MISEQ® Personal Sequencer (ILLUMINA®, Inc., San Diego, CA). The resulting reads were examined for the presence and nature of repairs at the expected sites of cleavage by comparison to control experiments where the Cas9 protein and guide RNA were omitted from the transfection or by comparison to the reference genome. To calculate the frequency of NHEJ mutations for a target site/Cas9 protein/guide RNA combination, the total number of mutant reads (amplicon sequences containing insertions or deletions when compared to the DNA sequences from control treatments or reference genome) was divided by total read number (wild-type plus mutant reads) of an appropriate length containing a perfect match to the barcode and forward primer. Total read counts averaged approximately 7000 per sample and NHEJ activity is expressed as the average (n=3) mutant fraction in Table 3.

A subset of gRNA pairs screened in porcine fetal fibroblasts were additionally tested for their ability to introduce a premature stop codon in the TMPRSS2 and TMPRSS4 coding regions in porcine embryos. Single guides whose repair outcomes generated in-frame stop codons in PFFs were also tested for their ability to do the same in porcine embryos. The subset of guides to be tested in porcine embryos was chosen based on their efficacy in generating premature stop codons in porcine fibroblasts. Edited porcine embryos were generated as described below. Briefly, oocytes recovered from slaughterhouse ovaries were in vitro fertilized. The sgRNP solution was injected into the cytoplasm of presumptive zygotes at 16-17 hours post-fertilization by using a single pulse from a FEMTOJET® 4i microinjector (Eppendorf; Hamburg, Germany). Microinjection was performed in TL-Hepes (ABT360, LLC) supplemented with 3 mg/ml BSA (Proliant) on the heated stage of an inverted microscope equipped with Narishige micromanipulators (Narishige International USA, Amityville, NY). Following injections, presumptive zygotes were cultured for 7 days in PZM5 (Cosmo Bio, Co LTD, Tokyo, Japan) in a 38.5° C. incubator environment of 5% CO₂, 5% O₂, 90% N₂. Mutation frequency of blastocysts was determined by ILLUMINA® sequencing as described for fetal fibroblasts above. The frequencies of the desired end-to-end NHEJ repairs resulting in premature stop codons in TMPRSS2 and TMPRSS4 are shown in Table 3.

This example demonstrates that the porcine TMPRSS2 and TMPRSS4 gene nucleotide sequences can be edited through the stimulation of double-strand breaks mediated by transfecting Cas9 protein with single or paired guide RNAs to effect a de novo in-frame stop codon.

TABLE 4
TMPRSS2 and TMPRSS4 Guides used to generate in-frame
stop codons in porcine fibroblasts and blastocysts

| | Guide 1 | Guide 2 | | |
|---|---|---|---|---|
| | SEQ ID | SEQ ID | % desired editing | % desired editing |
| Gene | No. | No. | fibroblasts | blastocysts |
| TMPRSS2 | 10 | 30 | 0 | 43 |
| TMPRSS4 | 203 | — | 7 | n.d. |
| TMPRSS4 | 274 | — | 2 | n.d. |
| TMPRSS4 | 196 | 203 | 32 | 24 |
| TMPRSS4 | 197 | 202 | 10 | n.d. |
| TMPRSS4 | 208 | 213 | 7 | n.d. |
| TMPRSS4 | 297 | 307 | 25 | 46 |
| TMPRSS4 | 297 | 308 | 13 | n.d. |
| TMPRSS4 | 297 | 312 | 7 | n.d. |
| TMPRSS4 | 328 | 336 | 0 | n.d. |
| TMPRSS4 | 328 | 338 | 32 | 31 |
| TMPRSS4 | 328 | 340 | 4 | n.d. |
| TMPRSS4 | 328 | 341 | 7 | n.d. |
| TMPRSS4 | 328 | 343 | 6 | n.d. |
| TMPRSS4 | 319 | 342 | 24 | n.d. |
| TMPRSS4 | 331 | 342 | 21 | n.d. |
| TMPRSS4 | 334 | 342 | 44 | 34 |
| TMPRSS4 | 336 | 342 | 0 | n.d. |

For TMPRSS2, the guides encoded by SEQ ID NOs 10 and 30 each cut, and then the blunt ends are joined through NHEJ to create SEQ ID NO: 445, which contains an exogenous stop codon at the ligated break-point.

For TMPRSS4, the guides encoded by SEQ ID NOs: 328 and 338 each cut, and then blunt ends are joined through NHEJ to create SEQ ID NO: 446, which contains an exogenous stop codon at the ligated break-point.

### Example 2: Generation of Pigs Having Edited TMPRSS2 and TMPRSS4 Genes

Pigs having an edit of TMPRSS2 alone, TMPRSS4 alone, or TMPRSS2 and TMPRSS4 will be generated.

Porcine oocytes will be isolated, fertilized, and then the resulting zygotes will be edited to generate gene edited pigs.

TMPRSS2 and TMPRSS4 guide RNA pairs selected from Table 3 will be used to form RNP complexes as described in Example 1 and will be microinjected into the cytoplasm of in vivo or in vitro fertilized PIC™ porcine one-cell zygotes or MII oocytes. These zygotes will then be incubated to generate edited multicellular embryos and transferred to surrogate gilts via standard methods to birth gene edited pigs. To prepare embryo donors and surrogates, PIC™ pubertal gilts will be subjected to estrus synchronization by treatment with 0.22% altrenogest solution (20-36 mg/animal) for 14 days. Follicular growth will be induced by the administration of PMSG 36 hours following the last dose of Matrix, and ovulation will be induced by the administration of hCG 82 hours after PMSG administration. To generate in vivo fertilized zygotes, females in standing heat will be artificially inseminated (AI) with PIC™ boar semen. In vivo derived zygotes will be recovered surgically 12-24 hours after AI by retrograde flushing the oviduct with sterile TL-HEPES medium supplemented with 0.3% BSA (w/v). Fertilized zygotes will be subjected to a single 2-50 picoliter (pl) cytoplasmic injection of Cas9 protein and guide RNA complex (25-50 ng/μl and 12.5-35 ng/μl) targeting TMPRSS2 and/or TMPRSS4 and cultured in PZM5 medium (Yoshioka, K., et al., Biol. Reprod., 2002, 60:112-119; Suzuki, C., et al., Reprod. Fertil. Dev., 2006 18, 789-795: Yoshioka, K., J. Reprod. Dev. 2008, 54, 208-213). Injected zygotes will be surgically implanted into the oviducts of estrus synchronized, un-mated surrogate females by a mid-line laparotomy under general anesthesia (each surrogate will receive 20-60 injected embryos).

In vitro fertilized embryos for gene editing will be derived from non-fertilized PIC™ oocytes. Immature oocytes from estrus synchronized PIC™ gilts will be collected from medium size (3-6 mm) follicles. Oocytes with evenly dark cytoplasm and intact surrounding cumulus cells will be selected for maturation. Cumulus oocyte complexes will be placed in a well containing 500 μl of maturation medium, TCM-199 (Invitrogen) with 3.05 mM glucose, 0.91 mM sodium pyruvate, 0.57 mM cysteine, 10 ng/ml EGF, 0.5 μg/ml luteinizing hormone (LH), 0.5 μg/ml FSH, 10 ng/ml gentamicin (Sigma), and 10% follicular fluid for 42-44 h at 38.5° C. and 5% CO₂, in humidified air. At the end of the maturation, the surrounding cumulus cells will be removed from the oocytes by vortexing for 3 min in the presence of 0.1% hyaluronidase. Then, in vitro matured oocytes will be placed in 100 μl droplets of IVF medium (modified Tris-buffered medium containing 113.1 mM NaCl, 3 mM KCl, 7.5 mM CaCl₂, 11 mM glucose, 20 mM Tris, 2 mM caffeine, 5 mM sodium pyruvate, and 2 mg/ml bovine serum albumin (BSA)) in groups of 25-30 oocytes and will be fertilized according to established protocol (Abeydeera, L. R. and Day, B. N., Biol. Reprod., 1997, 57, 729-734) using fresh extended boar semen. One ml of extended semen will be mixed with Dulbecco's Phosphate Buffered Saline (DPBS) containing 1 mg/ml BSA to a final volume of 10 ml and centrifuged at 1000×g, 25° C. for 4 minutes, and spermatozoa will be washed in DPBS three times. After the final wash, spermatozoa will be re-suspended in mTBM medium and added to oocytes at a final concentration of 1×10⁵ spermatozoa/ml, and co-incubated for 4-5 h at 38.5° C. and 5% CO₂. Presumptive zygotes will be microinjected 5 hours post IVF and transferred to a surrogate female after 18-42 hours (1-4 cell stage). Each surrogate receives 20-60 injected embryos. Pregnancies will be confirmed by lack of return to estrus (21 days) and ultrasound at 28 days post embryo transfer.

### Example 3: Molecular Characterization of Gene Edited Pigs (Sequencing)

A tissue sample will be taken from an animal with a genome edited according to the examples herein. Tail, ear notch, or blood samples are suitable tissue types. The tissue sample will be frozen at −20° C. within 1 hour of sampling to preserve integrity of the DNA in the tissue sample.

DNA will be extracted from tissue samples after proteinase K digestion in lysis buffer. Characterization will be performed on two different sequence platforms, short sequence reads using the ILLUMINA® platform (ILLUMINA®, San Diego, CA) and long sequence reads on the Oxford NANOPORE™ platform (Oxford NANOPORE™ Technologies, Oxford, UK).

For short sequence reads, two-step PCR will be used to amplify and sequence the region of interest. The first step is a locus-specific PCR which amplifies the locus of interest from the DNA sample using a combined locus-specific primer with a vendor-specific primer. The second step attaches the sequencing index and adaptor sequences to the amplicon from the first step so that sequencing can occur.

The locus-specific primers for the first step PCR are chosen so that they amplify a region<300 bp such that ILLUMINA® paired-end sequencing reads could span the amplified fragment. Multiple amplicons are preferred to provide redundancy should deletions or naturally occurring point mutations prevent primers from correctly binding. Sequence data for the amplicon will be generated using an ILLUMINA® sequencing platform (MISEQ®, ILLUMINA®, San Diego, CA). Sequence reads are analyzed to characterize the outcome of the editing process.

For long sequence reads, two-step PCR will be used to amplify and sequence the region of interest. The first step is a locus-specific PCR which amplifies the locus of interest from the DNA sample using a combined locus-specific primer with a vendor-specific adapter. The second step PCR attaches the sequencing index to the amplicon from the first-step PCR so that the DNA is ready for preparing a sequencing library. The step 2 PCR products undergo a set of chemical reactions from a vendor kit to polish the ends of the DNA and ligate on the adapter containing the motor protein to allow access to the pores for DNA strand-based sequencing.

The locus specific primers for the first step PCR range will be designed to amplify different regions of the TMPRSS2 and TMPRSS4 genes and will amplify regions different in length. Normalized DNA is then mixed with vendor supplied loading buffer and is loaded onto the NANOPORE™ flowcell.

Long sequence reads, while having lower per base accuracy than short reads, are very useful for observing the long range context of the sequence around the target site.

### Example 4: Influenza Challenge of TMPRSS2 and TMPRSS4 Edited Pigs

PIC™ pigs will be edited with guides as described in Example 2. Edits will be confirmed as described in Example 3. Edited pigs will then be crossbred to create pigs that are homozygous for the edit. These homozygous edited pigs will be inoculated with influenza virus. Viral titers will be determined as tissue culture infectious dose 50 (TCID₅₀) using standard methods known in the art. It is expected that the viral titers for edited pigs will be significantly lower than control pigs without the edit.

### Example 5 Assay for Real Time PCR Verification of Desired TMPRSS2 and TMPRSS4 Edits

There will be two assays created for detection of each of the TMPRSS2 and TMPRSS4 edits using real time PCR (rtPCR). For the first assay, two sets of primers and two probes will be designed. One set of primers will flank the spacer sequence of the first guide RNA (the first gRNA is SEQ ID NO: 10 for TMPRSS2 and SEQ ID NO: 328 for TMPRSS4). A probe, labeled with a fluorescent moiety, will be designed to anneal to the unedited version of the spacer sequence. The other set of primers will be designed to flank the desired edit sequence (the desired edit sequence is SEQ ID NO: 445 for TMPRS2 and SEQ ID NO: 446 for TMPRSS4). A probe, labeled with a different fluorescent moiety, will be designed to anneal to nucleotides spanning the joining region of the edit. For validation, real time PCR will be performed using a commercial kit using DNA isolated from pigs of confirmed status from sequencing, and fluorescence will be charted. Validation will occur if, as expected, the homozygotes are close to the y axis (representing the fluorescent moiety for the probe annealing to the desired edit), the heterozygotes group near the center of the chart, and the wild type pigs group close to the X axis (representing the fluorescent moiety for the probe annealing to the spacer sequence).

For the second assay, two sets of primers and two probes will also be designed. One set of primers will flank the second spacer sequence (the second spacer sequence is SEQ ID NO: 30 for TMPRSS2 and SEQ ID. NO: 338 for TMPRSS4). A probe, labeled with a fluorescent moiety, will be designed to anneal to the unedited version of the spacer sequence. The other set of primers will be designed to flank the desired edit sequence these may be the same primers and probe used for the first assay. A probe, labeled with a different fluorescent moiety, will be designed to anneal to nucleotides spanning the joining region of the edit. For validation, real time PCR will be performed using a commercial kit using DNA isolated from pigs of confirmed status from sequencing, and fluorescence will be charted. Validation will occur if, as expected, the homozygotes are close to the y axis (representing the fluorescent moiety for the probe annealing to the desired edit), the heterozygotes group near the center of the chart, and the wild type pigs group close to the X axis (representing the fluorescent moiety for the probe annealing to the spacer sequence).

### Example 6: PEDV Challenge of TMPRSS2 and TMPRSS4 Edited Pigs

PIC™ pigs will be edited with guides as described in Example 2. Edits will be confirmed as described in Example 3. Edited pigs will then be crossbred to create pigs that are homozygous for the edit. PEDV viral challenge of homozygous edited pigs will be performed as described in Whitworth, K. M., et al., Transgenic research, 28, 21-32. Briefly, homozygous edited pigs will be inoculated with PEDV. Fecal swabs will be collected at intervals beginning prior to inoculation. Realtime PCR to determine the presence of virus in the feces will be performed using standard methods (see e.g. Whitworth, K. M., et al., Transgenic research, 28, 21-32). It is expected that the viral amounts for edited pigs will be significantly lower than control pigs without the edit.

### Example 7: Influenza and PeDV Challenge of TMPRSS2 and TMPRSS4 Edited Pigs

PIC™ pigs will be edited with guides as described in Example 2. Edits will be confirmed as described in Example 3. Edited pigs will then be crossbred to create pigs that are homozygous for the edit. These homozygous edited pigs will be inoculated with influenza virus and PEDV as described in Sunwoo, S. Y., et al., Vaccines, 2018, 64 and Whitworth, K. M., et al., Transgenic research, 28, 21-32, respectively. Briefly, animals will be inoculated as described. Viral titers for influenza will be determined using TCID₅₀ in Bronchoalveolar lavage fluid (BALF) and lung lesions will also be scored (see, e.g. Example 10). For PEDV, realtime PCR to determine the presence of virus in fecal swabs (see Example 6) will be performed using standard methods (see, e.g. Whitworth, K. M., et al., Transgenic research, 28, 21-32). It is expected that the viral amounts for edited pigs will be significantly lower than control pigs without the edit.

### Example 8: TMPRSS Edit Resistance to PEDV Ex Vivo

A porcine alveolar macrophage line, 3D4, (Weingartl, H. M., et al., J. Virol. Methods, 2002, 104, 203-216) will be edited for TMPRSS2, TMPRSS4, or both using the SEQ ID NOs 10 and 30 guides for TMPRRS2 and SEQ ID NOs: 328 and 338 for TMPRSS4 using standard methods. Wild type 3D4 cells, TMPRSS 2 edit 3D4 cells. TMPRSS4 edit 3D4 cells, and TMPRSS2/4 edit 3D4 cells will be seeded into plates 24 hours prior to infection. Each culture will then be infected with PEDV at a constant MOI. Cells will be washed once after virus adsorption and then incubated at 37° C. for 5 days. Viral titer will be assayed at 6, 12, 24, 48, and 72 hours post infection. It is expected that TMPRSS knock-out cells will have lower viral titers than wild-type cells.

### Example 9: Trypsin Rescue of TMPRSS Knock-Out PEDV Infection

TMPRSS edited lines from Example 8 will be co-incubated with PEDV at a constant MOI and trypsin at 0.5 μg/mL, 10 μg/mL, 15 μg/mL, and 20 g/mL. Viral titers will be taken for each trypsin concentration. It is expected that sufficient amounts of trypsin in the culture will cause the TMPRSS gene edited cells to have viral titers closer to wild-type cells. Without being limited by theory, because trypsin is known to have serine protease activity, it is expected to substitute for the TMPRSS protein in cleaving viral HA, allowing the virus to infect the edited cells in the absence and/or reduction of TMPRSS activity.

### Example 10: Viral Challenge of TMPRSS4 Gene Edited Pigs

To generate edited pigs, PFFs were edited using guides of SEQ ID NOs: 328 and 338 as described in Example 1. DNA was extracted from the edited cells and verified using sequencing as described in Example 3. The verified cell lines were then utilized for SCNT by a commercial lab using established methods (RenOV Ate Biosciences, Inc., Maryland, USA). Five pigs carrying the TMPRSS4 edit and five wildtype control pigs were challenged intratracheally with H1N1 swine influenza isolate CA04 according to published methods (Sunwoo, S. Y., et al., Vaccines, 2018, 64) except that the pigs were challenged with 10⁶ TCID₅₀ of virus. All animals were humanely euthanized at 5 days post-challenge. Bronchoalveolar lavage fluid (BALF) was collected according to standard methods and influenza titers calculated as tissue culture infectious dose 50% (TCID₅₀) (see, e.g. Sunwoo, S. Y., et al., Vaccines, 2018, 64). Lung lesions were scored according to standard methods (see, e.g., Sunwoo, S. Y., et al., Vaccines, 2018, 64). BALF titers and lung scores were analyzed by one-sided Mann-Whitney nonparametric tests. The results are shown in Tables 5 and 6.

TABLE 5
Viral titers from bronchoalveolar lavage fluid by genotype.

| TMPRSS4 Genotype | Edited | Wild-type |
|---|---|---|
| Geometric Mean | 3570 | 316000 |
| BALF TCID50 | | |

TABLE 6
Macroscopic lung scores by genotype.

| TMPRSS4 Genotype | Edited | Wild-type |
|---|---|---|
| Mean Macroscopic Lung Score | 4.9% | 11.7% |

TMPRSS4 knockout pigs had more than 88-fold lower viral titers from lung lavage fluid than wildtype controls (P=0.018: Table 5). TMPRSS4 knockout pigs also had lower lung lesion scores than wildtype controls (P=0.048: Table 6). Thus, in addition to less virus the TMPRSS4 knockout pigs had less severe influenza disease pathology in pig lungs than wildtype controls. This example illustrates that disruption of the TMPRSS4 gene increases the resistance of pigs to influenza virus.

All references, including publications, patents, and patent applications, cited herein are hereby incorporated by reference to the extent they are not inconsistent with the explicit details of this disclosure, and are so incorporated to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention. Examples disclosed herein are provided by way of exemplification and are not intended to limit the scope of the invention.

1. A livestock animal comprising a gene edit in a TMPRSS4 gene wherein the livestock animal shows increased resistance to a virus relative to a wild type livestock animal.

2. The livestock animal according to claim 1, wherein the gene edit in the TMPRSS4 gene encodes a TMPRSS4 protein that exhibits reduced cleavage of a viral hemagglutinin (HA) protein and/or a coronavirus spike protein.

3. The livestock animal according to claim 1, wherein the gene edit in the TMPRSS4 gene comprises a premature stop codon.

4. The livestock animal according to claim 3, wherein the premature stop codon is in Exon 5, Exon 9 or Exon 10.

5. The livestock animal according to claim 1, wherein the gene edit in the TMPRSS4 gene comprises SEQ ID NO: 446.

6. (canceled)

7. The livestock animal according to claim 1, wherein the livestock animal is a pig.

8. The livestock animal according to claim 1, further comprising a gene edit in a TMPRSS2 gene.

9. The livestock animal according to claim 8, wherein the gene edit in the TMPRSS4 gene and the gene edit in the TMPRSS2 gene each comprise a premature stop codon.

10. The livestock animal according to claim 8, wherein the gene edit in the TMPRSS4 gene comprises a premature stop codon in Exon 5, Exon 9, or Exon 10 and the gene edit in the TMPRSS2 gene comprises a premature stop codon in Exon 3, Exon 5, or Exon 9.

11. (canceled)

12. (canceled)

13. The livestock animal according to claim 8, wherein the gene edit in the TMPRSS2 gene encodes a TMPRSS2 protein that exhibits reduced cleavage of a viral HA protein and/or a coronavirus spike protein.

14. The livestock animal according to claim 8, wherein the livestock animal is a pig.

15. (canceled)

16. A livestock animal comprising a gene edit in a TMPRSS2 gene wherein the gene edit affects the sequence of any one of exons 3-16.

17. The livestock animal according to claim 16, wherein the gene edit in a TMPRSS2 gene is in exon 3.

18. The livestock animal according to claim 16, wherein the gene edit in the TMPRSS2 gene encodes a TMPRRS2 protein that exhibits reduced cleavage of a viral HA protein and/or a coronavirus spike protein.

19. The livestock animal according to claim 16, wherein the gene edit in the TMPRSS2 gene comprises a premature stop codon.

20. The livestock animal according to claim 16, wherein the premature stop codon is located in Exon 3, Exon 5, or Exon 9.

21. (canceled)

22. The livestock animal according to claim 16, wherein the livestock animal is a pig.

23-31. (canceled)

32. A method of producing a gene edited livestock animal wherein the livestock animal shows increased resistance to a virus relative to a wild type livestock animal comprising:
i) introducing
a) a Cas9 protein or a nucleic acid encoding a Cas9 protein and
b) at least one guideRNA (gRNA) configured to create an edited TMPRSS2 sequence wherein the edited TMPRSS2 sequence comprises a premature stop codon downstream of nucleotide 14,361 as compared to SEQ ID NO: 437 into an isolated livestock animal cell;
ii) producing a livestock animal from the isolated livestock animal cell.

33-36. (canceled)

37. The method according to claim 32, wherein the gene edited livestock animal is a pig.

38. The method according to claim 32, further comprising mating the gene edited livestock animal to a livestock animal comprising a gene edited TMPRSS4 gene.


SEQUENCE LISTING (446 sequences)

SEQ ID NO: 1 - DNA, 23 bp, Sus scrofa
  source 1..23: mol_type = other DNA
aagagcacatcgtcctggatcgg

SEQ ID NO: 2 - DNA, 23 bp, Sus scrofa
  source 1..23: mol_type = other DNA
agagcacatcgtcctggatcggg

SEQ ID NO: 3 - DNA, 23 bp, Sus scrofa
  source 1..23: mol_type = other DNA
gagcacatcgtcctggatcgggg

SEQ ID NO: 4 - DNA, 23 bp, Sus scrofa
  source 1..23: mol_type = other DNA
catcgtcctggatcggggtttgg

SEQ ID NO: 5 - DNA, 23 bp, Sus scrofa
  source 1..23: mol_type = other DNA
atcgtcctggatcggggtttggg

SEQ ID NO: 6 - DNA, 23 bp, Sus scrofa
  source 1..23: mol_type = other DNA
ggcagcccaaaccccgatccagg

SEQ ID NO: 7 - DNA, 23 bp, Sus scrofa
  source 1..23: mol_type = other DNA
gggctgcctgctgacgacgacgg

SEQ ID NO: 8 - DNA, 23 bp, Sus scrofa
  source 1..23: mol_type = other DNA
ctgcctgctgacgacgacggcgg

SEQ ID NO: 9 - DNA, 23 bp, Sus scrofa
  source 1..23: mol_type = other DNA
tgcctgctgacgacgacggcggg

SEQ ID NO: 10 - DNA, 23 bp, Sus scrofa
  source 1..23: mol_type = other DNA
cacccgccgtcgtcgtcagcagg

SEQ ID NO: 11 - DNA, 23 bp, Sus scrofa
  source 1..23: mol_type = other DNA
gctgacgacgacggcgggtgtgg

SEQ ID NO: 12 - DNA, 23 bp, Sus scrofa
  source 1..23: mol_type = other DNA
gaagcgtgcgtctgcaccctcgg

SEQ ID NO: 13 - DNA, 23 bp, Sus scrofa
  source 1..23: mol_type = other DNA
aagcgtgcgtctgcaccctcggg

SEQ ID NO: 14 - DNA, 23 bp, Sus scrofa
  source 1..23: mol_type = other DNA
agcgtgcgtctgcaccctcgggg

SEQ ID NO: 15 - DNA, 23 bp, Sus scrofa
  source 1..23: mol_type = other DNA
tctgcaccctcggggcatactgg

SEQ ID NO: 16 - DNA, 23 bp, Sus scrofa
  source 1..23: mol_type = other DNA
ctgcaccctcggggcatactggg

SEQ ID NO: 17 - DNA, 23 bp, Sus scrofa
  source 1..23: mol_type = other DNA
tgcaccctcggggcatactgggg

SEQ ID NO: 18 - DNA, 23 bp, Sus scrofa
  source 1..23: mol_type = other DNA
gcaccctcggggcatactggggg

SEQ ID NO: 19 - DNA, 23 bp, Sus scrofa
  source 1..23: mol_type = other DNA
ggtcccccagtatgccccgaggg

SEQ ID NO: 20 - DNA, 23 bp, Sus scrofa
  source 1..23: mol_type = other DNA
cggtcccccagtatgccccgagg

SEQ ID NO: 21 - DNA, 23 bp, Sus scrofa
  source 1..23: mol_type = other DNA
cggggcatactgggggaccgagg

SEQ ID NO: 22 - DNA, 23 bp, Sus scrofa
  source 1..23: mol_type = other DNA
ggggcatactgggggaccgaggg

SEQ ID NO: 23 - DNA, 23 bp, Sus scrofa
  source 1..23: mol_type = other DNA
gcatactgggggaccgagggcgg

SEQ ID NO: 24 - DNA, 23 bp, Sus scrofa
  source 1..23: mol_type = other DNA
catactgggggaccgagggcggg

SEQ ID NO: 25 - DNA, 23 bp, Sus scrofa
  source 1..23: mol_type = other DNA
actgggggaccgagggcgggtgg

SEQ ID NO: 26 - DNA, 23 bp, Sus scrofa
  source 1..23: mol_type = other DNA
ggctcagtaccacccgccctcgg

SEQ ID NO: 27 - DNA, 23 bp, Sus scrofa
  source 1..23: mol_type = other DNA
gagggcgggtggtactgagccgg

SEQ ID NO: 28 - DNA, 23 bp, Sus scrofa
  source 1..23: mol_type = other DNA
ctgagccggatacgccccatagg

SEQ ID NO: 29 - DNA, 23 bp, Sus scrofa
  source 1..23: mol_type = other DNA
tgagccggatacgccccataggg

SEQ ID NO: 30 - DNA, 23 bp, Sus scrofa
  source 1..23: mol_type = other DNA
caggccctatggggcgtatccgg

SEQ ID NO: 31 - DNA, 23 bp, Sus scrofa
  source 1..23: mol_type = other DNA
gatacgccccatagggcctgtgg

SEQ ID NO: 32 - DNA, 23 bp, Sus scrofa
  source 1..23: mol_type = other DNA
atacgccccatagggcctgtggg

SEQ ID NO: 33 - DNA, 23 bp, Sus scrofa
  source 1..23: mol_type = other DNA
cggggcccacaggccctatgggg

SEQ ID NO: 34 - DNA, 23 bp, Sus scrofa
  source 1..23: mol_type = other DNA
cccatagggcctgtgggccccgg

SEQ ID NO: 35 - DNA, 23 bp, Sus scrofa
  source 1..23: mol_type = other DNA
ccggggcccacaggccctatggg

SEQ ID NO: 36 - DNA, 23 bp, Sus scrofa
  source 1..23: mol_type = other DNA
ccatagggcctgtgggccccggg

SEQ ID NO: 37 - DNA, 23 bp, Sus scrofa
  source 1..23: mol_type = other DNA
cccggggcccacaggccctatgg

SEQ ID NO: 38 - DNA, 23 bp, Sus scrofa
  source 1..23: mol_type = other DNA
catagggcctgtgggccccgggg

SEQ ID NO: 39 - DNA, 23 bp, Sus scrofa
  source 1..23: mol_type = other DNA
atagggcctgtgggccccggggg

SEQ ID NO: 40 - DNA, 23 bp, Sus scrofa
  source 1..23: mol_type = other DNA
tagggcctgtgggccccgggggg

SEQ ID NO: 41 - DNA, 23 bp, Sus scrofa
  source 1..23: mol_type = other DNA
cgcagccccccggggcccacagg

SEQ ID NO: 42 - DNA, 23 bp, Sus scrofa
  source 1..23: mol_type = other DNA
ctgtgggccccggggggctgcgg

SEQ ID NO: 43 - DNA, 23 bp, Sus scrofa
  source 1..23: mol_type = other DNA
tgtgggccccggggggctgcggg

SEQ ID NO: 44 - DNA, 23 bp, Sus scrofa
  source 1..23: mol_type = other DNA
gtgggccccggggggctgcgggg

SEQ ID NO: 45 - DNA, 23 bp, Sus scrofa
  source 1..23: mol_type = other DNA
tgggccccggggggctgcggggg

SEQ ID NO: 46 - DNA, 23 bp, Sus scrofa
  source 1..23: mol_type = other DNA
gggccccggggggctgcgggggg

SEQ ID NO: 47 - DNA, 23 bp, Sus scrofa
  source 1..23: mol_type = other DNA
ctaccccccgcagccccccgggg

SEQ ID NO: 48 - DNA, 23 bp, Sus scrofa
  source 1..23: mol_type = other DNA
tctaccccccgcagccccccggg

SEQ ID NO: 49 - DNA, 23 bp, Sus scrofa
  source 1..23: mol_type = other DNA
gtctaccccccgcagccccccgg

SEQ ID NO: 50 - DNA, 23 bp, Sus scrofa
  source 1..23: mol_type = other DNA
tgcggggggtagacactttcagg

SEQ ID NO: 51 - DNA, 23 bp, Sus scrofa
  source 1..23: mol_type = other DNA
gggggtagacactttcaggttgg

SEQ ID NO: 52 - DNA, 23 bp, Sus scrofa
  source 1..23: mol_type = other DNA
cactttcaggttggtatccgtgg

SEQ ID NO: 53 - DNA, 23 bp, Sus scrofa
  source 1..23: mol_type = other DNA
tatccgtggttttcatagtaagg

SEQ ID NO: 54 - DNA, 23 bp, Sus scrofa
  source 1..23: mol_type = other DNA
gggccttactatgaaaaccacgg

SEQ ID NO: 55 - DNA, 23 bp, Sus scrofa
  source 1..23: mol_type = other DNA
tcatagtaaggcccgacacctgg

SEQ ID NO: 56 - DNA, 23 bp, Sus scrofa
  source 1..23: mol_type = other DNA
ggcgaggacgagaaccagtgtgg

SEQ ID NO: 57 - DNA, 23 bp, Sus scrofa
  source 1..23: mol_type = other DNA
ctggttctcgtcctcgccgccgg

SEQ ID NO: 58 - DNA, 23 bp, Sus scrofa
  source 1..23: mol_type = other DNA
tggttctcgtcctcgccgccggg

SEQ ID NO: 59 - DNA, 23 bp, Sus scrofa
  source 1..23: mol_type = other DNA
cctcgccgccgggacagtgcagg

SEQ ID NO: 60 - DNA, 23 bp, Sus scrofa
  source 1..23: mol_type = other DNA
cctgcactgtcccggcggcgagg

SEQ ID NO: 61 - DNA, 23 bp, Sus scrofa
  source 1..23: mol_type = other DNA
ggcatcctgcactgtcccggcgg

SEQ ID NO: 62 - DNA, 23 bp, Sus scrofa
  source 1..23: mol_type = other DNA
gatggcatcctgcactgtcccgg

SEQ ID NO: 63 - DNA, 23 bp, Sus scrofa
  source 1..23: mol_type = other DNA
gatgccatcgcaccagtgagagg

SEQ ID NO: 64 - DNA, 23 bp, Sus scrofa
  source 1..23: mol_type = other DNA
atgccatcgcaccagtgagaggg

SEQ ID NO: 65 - DNA, 23 bp, Sus scrofa
  source 1..23: mol_type = other DNA
tgccatcgcaccagtgagagggg

SEQ ID NO: 66 - DNA, 23 bp, Sus scrofa
  source 1..23: mol_type = other DNA
agcccctctcactggtgcgatgg

SEQ ID NO: 67 - DNA, 23 bp, Sus scrofa
  source 1..23: mol_type = other DNA
ccagtgagaggggctgatgcagg

SEQ ID NO: 68 - DNA, 23 bp, Sus scrofa
  source 1..23: mol_type = other DNA
cctgcatcagcccctctcactgg

SEQ ID NO: 69 - DNA, 23 bp, Sus scrofa
  source 1..23: mol_type = other DNA
agatggagtgcggctcttctggg

SEQ ID NO: 70 - DNA, 23 bp, Sus scrofa
  source 1..23: mol_type = other DNA
gagatggagtgcggctcttctgg

SEQ ID NO: 71 - DNA, 23 bp, Sus scrofa
  source 1..23: mol_type = other DNA
agaagagccgcactccatctcgg

SEQ ID NO: 72 - DNA, 23 bp, Sus scrofa
  source 1..23: mol_type = other DNA
gaagagccgcactccatctcggg

SEQ ID NO: 73 - DNA, 23 bp, Sus scrofa
  source 1..23: mol_type = other DNA
gccgcactccatctcgggcgtgg

SEQ ID NO: 74 - DNA, 23 bp, Sus scrofa
  source 1..23: mol_type = other DNA
tccacgcccgagatggagtgcgg

SEQ ID NO: 75 - DNA, 23 bp, Sus scrofa
  source 1..23: mol_type = other DNA
gaggtgctccacgcccgagatgg

SEQ ID NO: 76 - DNA, 23 bp, Sus scrofa
  source 1..23: mol_type = other DNA
ctctgcaagtggagaagaagagg

SEQ ID NO: 77 - DNA, 23 bp, Sus scrofa
  source 1..23: mol_type = other DNA
ctcttcttctccacttgcagagg

SEQ ID NO: 78 - DNA, 23 bp, Sus scrofa
  source 1..23: mol_type = other DNA
tcttcttctccacttgcagaggg

SEQ ID NO: 79 - DNA, 23 bp, Sus scrofa
  source 1..23: mol_type = other DNA
cttctccacttgcagagggacgg

SEQ ID NO: 80 - DNA, 23 bp, Sus scrofa
  source 1..23: mol_type = other DNA
ttctccacttgcagagggacggg

SEQ ID NO: 81 - DNA, 23 bp, Sus scrofa
  source 1..23: mol_type = other DNA
tccacttgcagagggacgggagg

SEQ ID NO: 82 - DNA, 23 bp, Sus scrofa
  source 1..23: mol_type = other DNA
ccacttgcagagggacgggaggg

SEQ ID NO: 83 - DNA, 23 bp, Sus scrofa
  source 1..23: mol_type = other DNA
ccctcccgtccctctgcaagtgg

SEQ ID NO: 84 - DNA, 23 bp, Sus scrofa
  source 1..23: mol_type = other DNA
gacggccgcccactgcgtggagg

SEQ ID NO: 85 - DNA, 23 bp, Sus scrofa
  source 1..23: mol_type = other DNA
tgtgacggccgcccactgcgtgg

SEQ ID NO: 86 - DNA, 23 bp, Sus scrofa
  source 1..23: mol_type = other DNA
ggcggccgtcacaatccagtcgg

SEQ ID NO: 87 - DNA, 23 bp, Sus scrofa
  source 1..23: mol_type = other DNA
gcggccgtcacaatccagtcggg

SEQ ID NO: 88 - DNA, 23 bp, Sus scrofa
  source 1..23: mol_type = other DNA
cggccgtcacaatccagtcgggg

SEQ ID NO: 89 - DNA, 23 bp, Sus scrofa
  source 1..23: mol_type = other DNA
ggccgtcacaatccagtcggggg

SEQ ID NO: 90 - DNA, 23 bp, Sus scrofa
  source 1..23: mol_type = other DNA
cacccccgactggattgtgacgg

SEQ ID NO: 91 - DNA, 23 bp, Sus scrofa
  source 1..23: mol_type = other DNA
aatccagtcgggggtgatgatgg

SEQ ID NO: 92 - DNA, 23 bp, Sus scrofa
  source 1..23: mol_type = other DNA
gctccatcatcacccccgactgg

SEQ ID NO: 93 - DNA, 23 bp, Sus scrofa
  source 1..23: mol_type = other DNA
tgatggagcctccgcagatgtgg

SEQ ID NO: 94 - DNA, 23 bp, Sus scrofa
  source 1..23: mol_type = other DNA
cagggcatccacatctgcggagg

SEQ ID NO: 95 - DNA, 23 bp, Sus scrofa
  source 1..23: mol_type = other DNA
ctccgcagatgtggatgccctgg

SEQ ID NO: 96 - DNA, 23 bp, Sus scrofa
  source 1..23: mol_type = other DNA
gtccagggcatccacatctgcgg

SEQ ID NO: 97 - DNA, 23 bp, Sus scrofa
  source 1..23: mol_type = other DNA
tgtggatgccctggacgtgcagg

SEQ ID NO: 98 - DNA, 23 bp, Sus scrofa
  source 1..23: mol_type = other DNA
caggtcagcctgcacgtccaggg

SEQ ID NO: 99 - DNA, 23 bp, Sus scrofa
  source 1..23: mol_type = other DNA
gcaggtcagcctgcacgtccagg

SEQ ID NO: 100 - DNA, 23 bp, Sus scrofa
  source 1..23: mol_type = other DNA
gacgtgcaggctgacctgccagg

SEQ ID NO: 101 - DNA, 23 bp, Sus scrofa
  source 1..23: mol_type = other DNA
acgtgcaggctgacctgccaggg

SEQ ID NO: 102 - DNA, 23 bp, Sus scrofa
  source 1..23: mol_type = other DNA
gctgggcgactggccctggcagg

SEQ ID NO: 103 - DNA, 23 bp, Sus scrofa
  source 1..23: mol_type = other DNA
cagcgctgggcgactggccctgg

SEQ ID NO: 104 - DNA, 23 bp, Sus scrofa
  source 1..23: mol_type = other DNA
ccagtcgcccagcgctgcgctgg

SEQ ID NO: 105 - DNA, 23 bp, Sus scrofa
  source 1..23: mol_type = other DNA
ccagcgcagcgctgggcgactgg

SEQ ID NO: 106 - DNA, 23 bp, Sus scrofa
  source 1..23: mol_type = other DNA
ggcgggtccagcgcagcgctggg

SEQ ID NO: 107 - DNA, 23 bp, Sus scrofa
  source 1..23: mol_type = other DNA
gggcgggtccagcgcagcgctgg

SEQ ID NO: 108 - DNA, 23 bp, Sus scrofa
  source 1..23: mol_type = other DNA
cgctggacccgcccacgatccgg

SEQ ID NO: 109 - DNA, 23 bp, Sus scrofa
  source 1..23: mol_type = other DNA
acccgcccacgatccggctctgg

SEQ ID NO: 110 - DNA, 23 bp, Sus scrofa
  source 1..23: mol_type = other DNA
gccagagccggatcgtgggcggg

SEQ ID NO: 111 - DNA, 23 bp, Sus scrofa
  source 1..23: mol_type = other DNA
cgccagagccggatcgtgggcgg

SEQ ID NO: 112 - DNA, 23 bp, Sus scrofa
  source 1..23: mol_type = other DNA
cgcccacgatccggctctggcgg

SEQ ID NO: 113 - DNA, 23 bp, Sus scrofa
  source 1..23: mol_type = other DNA
aaccgccagagccggatcgtggg

SEQ ID NO: 114 - DNA, 23 bp, Sus scrofa
  source 1..23: mol_type = other DNA
caaccgccagagccggatcgtgg

SEQ ID NO: 115 - DNA, 23 bp, Sus scrofa
  source 1..23: mol_type = other DNA
agatgagcaaccgccagagccgg

SEQ ID NO: 116 - DNA, 23 bp, Sus scrofa
  source 1..23: mol_type = other DNA
ctggcggttgctcatcttcccgg

SEQ ID NO: 117 - DNA, 23 bp, Sus scrofa
  source 1..23: mol_type = other DNA
ctttagagtgcggagtctccggg

SEQ ID NO: 118 - DNA, 23 bp, Sus scrofa
  source 1..23: mol_type = other DNA
cctttagagtgcggagtctccgg

SEQ ID NO: 119 - DNA, 23 bp, Sus scrofa
  source 1..23: mol_type = other DNA
ggcttgtcgcctttagagtgcgg

SEQ ID NO: 120 - DNA, 25 bp, Sus scrofa
  source 1..25: mol_type = other DNA
aagagcacatcgtcctggatcgggg

SEQ ID NO: 121 - DNA, 25 bp, Sus scrofa
  source 1..25: mol_type = other DNA
gggctgcctgctgacgacgacggcg

SEQ ID NO: 122 - DNA, 25 bp, Sus scrofa
  source 1..25: mol_type = other DNA
tgcctgctgacgacgacggcgggtg

SEQ ID NO: 123 - DNA, 25 bp, Sus scrofa
  source 1..25: mol_type = other DNA
gaagcgtgcgtctgcaccctcgggg

SEQ ID NO: 124 - DNA, 25 bp, Sus scrofa
  source 1..25: mol_type = other DNA
tctgcaccctcggggcatactgggg

SEQ ID NO: 125 - DNA, 25 bp, Sus scrofa
  source 1..25: mol_type = other DNA
ctgcaccctcggggcatactggggg

SEQ ID NO: 126 - DNA, 25 bp, Sus scrofa
  source 1..25: mol_type = other DNA
ggtcccccagtatgccccgagggtg

SEQ ID NO: 127 - DNA, 25 bp, Sus scrofa
  source 1..25: mol_type = other DNA
ggggcatactgggggaccgagggcg

SEQ ID NO: 128 - DNA, 25 bp, Sus scrofa
  source 1..25: mol_type = other DNA
catactgggggaccgagggcgggtg

SEQ ID NO: 129 - DNA, 25 bp, Sus scrofa
  source 1..25: mol_type = other DNA
cggggcccacaggccctatggggcg

SEQ ID NO: 130 - DNA, 25 bp, Sus scrofa
  source 1..25: mol_type = other DNA
cccggggcccacaggccctatgggg

SEQ ID NO: 131 - DNA, 25 bp, Sus scrofa
  source 1..25: mol_type = other DNA
cccatagggcctgtgggccccgggg

SEQ ID NO: 132 - DNA, 25 bp, Sus scrofa
  source 1..25: mol_type = other DNA
ccatagggcctgtgggccccggggg

SEQ ID NO: 133 - DNA, 25 bp, Sus scrofa
  source 1..25: mol_type = other DNA
catagggcctgtgggccccgggggg

SEQ ID NO: 134 - DNA, 25 bp, Sus scrofa
  source 1..25: mol_type = other DNA
ctgtgggccccggggggctgcgggg

SEQ ID NO: 135 - DNA, 25 bp, Sus scrofa
  source 1..25: mol_type = other DNA
tgtgggccccggggggctgcggggg

SEQ ID NO: 136 - DNA, 25 bp, Sus scrofa
  source 1..25: mol_type = other DNA
gtgggccccggggggctgcgggggg

SEQ ID NO: 137 - DNA, 25 bp, Sus scrofa
  source 1..25: mol_type = other DNA
gtctaccccccgcagccccccgggg

SEQ ID NO: 138 - DNA, 25 bp, Sus scrofa
  source 1..25: mol_type = other DNA
ggcatcctgcactgtcccggcggcg

SEQ ID NO: 139 - DNA, 25 bp, Sus scrofa
  source 1..25: mol_type = other DNA
gatggcatcctgcactgtcccggcg

SEQ ID NO: 140 - DNA, 25 bp, Sus scrofa
  source 1..25: mol_type = other DNA
gatgccatcgcaccagtgagagggg

SEQ ID NO: 141 - DNA, 25 bp, Sus scrofa
  source 1..25: mol_type = other DNA
cctgcatcagcccctctcactggtg

SEQ ID NO: 142 - DNA, 25 bp, Sus scrofa
  source 1..25: mol_type = other DNA
gaagagccgcactccatctcgggcg

SEQ ID NO: 143 - DNA, 25 bp, Sus scrofa
  source 1..25: mol_type = other DNA
gccgcactccatctcgggcgtggag

SEQ ID NO: 144 - DNA, 25 bp, Sus scrofa
  source 1..25: mol_type = other DNA
gaggtgctccacgcccgagatggag

SEQ ID NO: 145 - DNA, 25 bp, Sus scrofa
  source 1..25: mol_type = other DNA
ctctgcaagtggagaagaagaggtg

SEQ ID NO: 146 - DNA, 25 bp, Sus scrofa
  source 1..25: mol_type = other DNA
ttctccacttgcagagggacgggag

SEQ ID NO: 147 - DNA, 25 bp, Sus scrofa
  source 1..25: mol_type = other DNA
ccctcccgtccctctgcaagtggag

SEQ ID NO: 148 - DNA, 25 bp, Sus scrofa
  source 1..25: mol_type = other DNA
ccacttgcagagggacgggagggag

SEQ ID NO: 149 - DNA, 25 bp, Sus scrofa
  source 1..25: mol_type = other DNA
gacggccgcccactgcgtggaggag

SEQ ID NO: 150 - DNA, 25 bp, Sus scrofa
  source 1..25: mol_type = other DNA
tgtgacggccgcccactgcgtggag

SEQ ID NO: 151 - DNA, 25 bp, Sus scrofa
  source 1..25: mol_type = other DNA
ggcggccgtcacaatccagtcgggg

SEQ ID NO: 152 - DNA, 25 bp, Sus scrofa
  source 1..25: mol_type = other DNA
gcggccgtcacaatccagtcggggg

SEQ ID NO: 153 - DNA, 25 bp, Sus scrofa
  source 1..25: mol_type = other DNA
ggccgtcacaatccagtcgggggtg

SEQ ID NO: 154 - DNA, 25 bp, Sus scrofa
  source 1..25: mol_type = other DNA
aatccagtcgggggtgatgatggag

SEQ ID NO: 155 - DNA, 25 bp, Sus scrofa
  source 1..25: mol_type = other DNA
gtccagggcatccacatctgcggag

SEQ ID NO: 156 - DNA, 25 bp, Sus scrofa
  source 1..25: mol_type = other DNA
ggcgggtccagcgcagcgctgggcg

SEQ ID NO: 157 - DNA, 25 bp, Sus scrofa
  source 1..25: mol_type = other DNA
acccgcccacgatccggctctggcg

SEQ ID NO: 158 - DNA, 25 bp, Sus scrofa
  source 1..25: mol_type = other DNA
aaccgccagagccggatcgtgggcg

SEQ ID NO: 159 - DNA, 25 bp, Sus scrofa
  source 1..25: mol_type = other DNA
ctggcggttgctcatcttcccggag

SEQ ID NO: 160 - DNA, 25 bp, Sus scrofa
  source 1..25: mol_type = other DNA
ggcttgtcgcctttagagtgcggag

SEQ ID NO: 161 - DNA, 23 bp, Sus scrofa
  source 1..23: mol_type = other DNA
ttcaggggttgatcactggcggg

SEQ ID NO: 162 - DNA, 23 bp, Sus scrofa
  source 1..23: mol_type = other DNA
gttcaggggttgatcactggcgg

SEQ ID NO: 163 - DNA, 23 bp, Sus scrofa
  source 1..23: mol_type = other DNA
gctgttcaggggttgatcactgg

SEQ ID NO: 164 - DNA, 23 bp, Sus scrofa
  source 1..23: mol_type = other DNA
gatcaacccctgaacagccttgg

SEQ ID NO: 165 - DNA, 23 bp, Sus scrofa
  source 1..23: mol_type = other DNA
agcttaccaaggctgttcagggg

SEQ ID NO: 166 - DNA, 23 bp, Sus scrofa
  source 1..23: mol_type = other DNA
gagcttaccaaggctgttcaggg

SEQ ID NO: 167 - DNA, 23 bp, Sus scrofa
  source 1..23: mol_type = other DNA
tgagcttaccaaggctgttcagg

SEQ ID NO: 168 - DNA, 23 bp, Sus scrofa
  source 1..23: mol_type = other DNA
ggtgcggggtttgcgctgggggg

SEQ ID NO: 169 - DNA, 23 bp, Sus scrofa
  source 1..23: mol_type = other DNA
gggtgcggggtttgcgctggggg

SEQ ID NO: 170 - DNA, 23 bp, Sus scrofa
  source 1..23: mol_type = other DNA
ggggtgcggggtttgcgctgggg

SEQ ID NO: 171 - DNA, 23 bp, Sus scrofa
  source 1..23: mol_type = other DNA
gggggtgcggggtttgcgctggg

SEQ ID NO: 172 - DNA, 23 bp, Sus scrofa
  source 1..23: mol_type = other DNA
ggggggtgcggggtttgcgctgg

SEQ ID NO: 173 - DNA, 23 bp, Sus scrofa
  source 1..23: mol_type = other DNA
aaggtctgcaggggggtgcgggg

SEQ ID NO: 174 - DNA, 23 bp, Sus scrofa
  source 1..23: mol_type = other DNA
gaaggtctgcaggggggtgcggg

SEQ ID NO: 175 - DNA, 23 bp, Sus scrofa
  source 1..23: mol_type = other DNA
tgaaggtctgcaggggggtgcgg

SEQ ID NO: 176 - DNA, 23 bp, Sus scrofa
  source 1..23: mol_type = other DNA
ctttttgaaggtctgcagggggg

SEQ ID NO: 177 - DNA, 23 bp, Sus scrofa
  source 1..23: mol_type = other DNA
ccccctgcagaccttcaaaaagg

SEQ ID NO: 178 - DNA, 23 bp, Sus scrofa
  source 1..23: mol_type = other DNA
cctttttgaaggtctgcaggggg

SEQ ID NO: 179 - DNA, 23 bp, Sus scrofa
  source 1..23: mol_type = other DNA
acctttttgaaggtctgcagggg

SEQ ID NO: 180 - DNA, 23 bp, Sus scrofa
  source 1..23: mol_type = other DNA
cacctttttgaaggtctgcaggg

SEQ ID NO: 181 - DNA, 23 bp, Sus scrofa
  source 1..23: mol_type = other DNA
cctgcagaccttcaaaaaggtgg

SEQ ID NO: 182 - DNA, 23 bp, Sus scrofa
  source 1..23: mol_type = other DNA
ccacctttttgaaggtctgcagg

SEQ ID NO: 183 - DNA, 23 bp, Sus scrofa
  source 1..23: mol_type = other DNA
ctgcagaccttcaaaaaggtggg

SEQ ID NO: 184 - DNA, 23 bp, Sus scrofa
  source 1..23: mol_type = other DNA
tgcagaccttcaaaaaggtgggg

SEQ ID NO: 185 - DNA, 23 bp, Sus scrofa
  source 1..23: mol_type = other DNA
ggggatccccacctttttgaagg

SEQ ID NO: 186 - DNA, 23 bp, Sus scrofa
  source 1..23: mol_type = other DNA
ggtggggatccccatcatcgcgg

SEQ ID NO: 187 - DNA, 23 bp, Sus scrofa
  source 1..23: mol_type = other DNA
ccccatcatcgcggcactgctgg

SEQ ID NO: 188 - DNA, 23 bp, Sus scrofa
  source 1..23: mol_type = other DNA
ccagcagtgccgcgatgatgggg

SEQ ID NO: 189 - DNA, 23 bp, Sus scrofa
  source 1..23: mol_type = other DNA
cccatcatcgcggcactgctggg

SEQ ID NO: 190 - DNA, 23 bp, Sus scrofa
  source 1..23: mol_type = other DNA
cccagcagtgccgcgatgatggg

SEQ ID NO: 191 - DNA, 23 bp, Sus scrofa
  source 1..23: mol_type = other DNA
gcccagcagtgccgcgatgatgg

SEQ ID NO: 192 - DNA, 23 bp, Sus scrofa
  source 1..23: mol_type = other DNA
catcgcggcactgctgggcctgg

SEQ ID NO: 193 - DNA, 23 bp, Sus scrofa
  source 1..23: mol_type = other DNA
cctggcagccatcgtcgtcgtgg

SEQ ID NO: 194 - DNA, 23 bp, Sus scrofa
  source 1..23: mol_type = other DNA
ccacgacgacgatggctgccagg

SEQ ID NO: 195 - DNA, 23 bp, Sus scrofa
  source 1..23: mol_type = other DNA
gagaatagccacgacgacgatgg

SEQ ID NO: 196 - DNA, 23 bp, Sus scrofa
  source 1..23: mol_type = other DNA
acaaatactacttcctctgctgg

SEQ ID NO: 197 - DNA, 23 bp, Sus scrofa
  source 1..23: mol_type = other DNA
agtggaggggctgccagcagagg

SEQ ID NO: 198 - DNA, 23 bp, Sus scrofa
  source 1..23: mol_type = other DNA
tctctcgggatgaagtggagggg

SEQ ID NO: 199 - DNA, 23 bp, Sus scrofa
  source 1..23: mol_type = other DNA
ctctctcgggatgaagtggaggg

SEQ ID NO: 200 - DNA, 23 bp, Sus scrofa
  source 1..23: mol_type = other DNA
gctctctcgggatgaagtggagg

SEQ ID NO: 201 - DNA, 23 bp, Sus scrofa
  source 1..23: mol_type = other DNA
gctgctctctcgggatgaagtgg

SEQ ID NO: 202 - DNA, 23 bp, Sus scrofa
  source 1..23: mol_type = other DNA
cgtcacacagctgctctctcggg

SEQ ID NO: 203 - DNA, 23 bp, Sus scrofa
  source 1..23: mol_type = other DNA
ccgagagagcagctgtgtgacgg

SEQ ID NO: 204 - DNA, 23 bp, Sus scrofa
  source 1..23: mol_type = other DNA
ccgtcacacagctgctctctcgg

SEQ ID NO: 205 - DNA, 23 bp, Sus scrofa
  source 1..23: mol_type = other DNA
gcagctgtgtgacggccagcagg

SEQ ID NO: 206 - DNA, 23 bp, Sus scrofa
  source 1..23: mol_type = other DNA
ggccagcaggactgtgcctcagg

SEQ ID NO: 207 - DNA, 23 bp, Sus scrofa
  source 1..23: mol_type = other DNA
gccagcaggactgtgcctcaggg

SEQ ID NO: 208 - DNA, 23 bp, Sus scrofa
  source 1..23: mol_type = other DNA
ccagcaggactgtgcctcagggg

SEQ ID NO: 209 - DNA, 23 bp, Sus scrofa
  source 1..23: mol_type = other DNA
cccctgaggcacagtcctgctgg

SEQ ID NO: 210 - DNA, 23 bp, Sus scrofa
  source 1..23: mol_type = other DNA
gcaggactgtgcctcaggggagg

SEQ ID NO: 211 - DNA, 23 bp, Sus scrofa
  source 1..23: mol_type = other DNA
gagctgctcatcctcccctgagg

SEQ ID NO: 212 - DNA, 23 bp, Sus scrofa
  source 1..23: mol_type = other DNA
tgtgtcaagaaattccccgatgg

SEQ ID NO: 213 - DNA, 23 bp, Sus scrofa
  source 1..23: mol_type = other DNA
gtgtcaagaaattccccgatggg

SEQ ID NO: 214 - DNA, 23 bp, Sus scrofa
  source 1..23: mol_type = other DNA
attccccgatgggcccccagtgg

SEQ ID NO: 215 - DNA, 23 bp, Sus scrofa
  source 1..23: mol_type = other DNA
ctgccactgggggcccatcgggg

SEQ ID NO: 216 - DNA, 23 bp, Sus scrofa
  source 1..23: mol_type = other DNA
cccgatgggcccccagtggcagg

SEQ ID NO: 217 - DNA, 23 bp, Sus scrofa
  source 1..23: mol_type = other DNA
cctgccactgggggcccatcggg

SEQ ID NO: 218 - DNA, 23 bp, Sus scrofa
  source 1..23: mol_type = other DNA
acctgccactgggggcccatcgg

SEQ ID NO: 219 - DNA, 23 bp, Sus scrofa
  source 1..23: mol_type = other DNA
ctggactcacctgccactggggg

SEQ ID NO: 220 - DNA, 23 bp, Sus scrofa
  source 1..23: mol_type = other DNA
cctggactcacctgccactgggg

SEQ ID NO: 221 - DNA, 23 bp, Sus scrofa
  source 1..23: mol_type = other DNA
ccctggactcacctgccactggg

SEQ ID NO: 222 - DNA, 23 bp, Sus scrofa
  source 1..23: mol_type = other DNA
cccctggactcacctgccactgg

SEQ ID NO: 223 - DNA, 25 bp, Sus scrofa
  source 1..25: mol_type = other DNA
gctgttcaggggttgatcactggcg

SEQ ID NO: 224 - DNA, 25 bp, Sus scrofa
  source 1..25: mol_type = other DNA
tgagcttaccaaggctgttcagggg

SEQ ID NO: 225 - DNA, 25 bp, Sus scrofa
  source 1..25: mol_type = other DNA
ggtgcggggtttgcgctggggggcg

SEQ ID NO: 226 - DNA, 25 bp, Sus scrofa
  source 1..25: mol_type = other DNA
ggggtgcggggtttgcgctgggggg

SEQ ID NO: 227 - DNA, 25 bp, Sus scrofa
  source 1..25: mol_type = other DNA
gggggtgcggggtttgcgctggggg

SEQ ID NO: 228 - DNA, 25 bp, Sus scrofa
  source 1..25: mol_type = other DNA
ggggggtgcggggtttgcgctgggg

SEQ ID NO: 229 - DNA, 25 bp, Sus scrofa
  source 1..25: mol_type = other DNA
tgaaggtctgcaggggggtgcgggg

SEQ ID NO: 230 - DNA, 25 bp, Sus scrofa
  source 1..25: mol_type = other DNA
ctttttgaaggtctgcaggggggtg

SEQ ID NO: 231 - DNA, 25 bp, Sus scrofa
  source 1..25: mol_type = other DNA
acctttttgaaggtctgcagggggg

SEQ ID NO: 232 - DNA, 25 bp, Sus scrofa
  source 1..25: mol_type = other DNA
ccccctgcagaccttcaaaaaggtg

SEQ ID NO: 233 - DNA, 25 bp, Sus scrofa
  source 1..25: mol_type = other DNA
cacctttttgaaggtctgcaggggg

SEQ ID NO: 234 - DNA, 25 bp, Sus scrofa
  source 1..25: mol_type = other DNA
ccacctttttgaaggtctgcagggg

SEQ ID NO: 235 - DNA, 25 bp, Sus scrofa
  source 1..25: mol_type = other DNA
cctgcagaccttcaaaaaggtgggg

SEQ ID NO: 236 - DNA, 25 bp, Sus scrofa
  source 1..25: mol_type = other DNA
gcccagcagtgccgcgatgatgggg

SEQ ID NO: 237 - DNA, 25 bp, Sus scrofa
  source 1..25: mol_type = other DNA
gctctctcgggatgaagtggagggg

SEQ ID NO: 238 - DNA, 25 bp, Sus scrofa
  source 1..25: mol_type = other DNA
gctgctctctcgggatgaagtggag

SEQ ID NO: 239 - DNA, 25 bp, Sus scrofa
  source 1..25: mol_type = other DNA
ggccagcaggactgtgcctcagggg

SEQ ID NO: 240 - DNA, 25 bp, Sus scrofa
  source 1..25: mol_type = other DNA
ccagcaggactgtgcctcaggggag

SEQ ID NO: 241 - DNA, 25 bp, Sus scrofa
  source 1..25: mol_type = other DNA
acctgccactgggggcccatcgggg

SEQ ID NO: 242 - DNA, 25 bp, Sus scrofa
  source 1..25: mol_type = other DNA
cccgatgggcccccagtggcaggtg

SEQ ID NO: 243 - DNA, 25 bp, Sus scrofa
  source 1..25: mol_type = other DNA
ccctggactcacctgccactggggg

SEQ ID NO: 244 - DNA, 25 bp, Sus scrofa
  source 1..25: mol_type = other DNA
cccctggactcacctgccactgggg

SEQ ID NO: 245 - DNA, 23 bp, Sus scrofa
  source 1..23: mol_type = other DNA
caggagagactgagggtctccgg

SEQ ID NO: 246 - DNA, 23 bp, Sus scrofa
  source 1..23: mol_type = other DNA
cggagaccctcagtctctcctgg

SEQ ID NO: 247 - DNA, 23 bp, Sus scrofa
  source 1..23: mol_type = other DNA
attattccaggagagactgaggg

SEQ ID NO: 248 - DNA, 23 bp, Sus scrofa
  source 1..23: mol_type = other DNA
aattattccaggagagactgagg

SEQ ID NO: 249 - DNA, 23 bp, Sus scrofa
  source 1..23: mol_type = other DNA
gggcaatgatgaattattccagg

SEQ ID NO: 250 - DNA, 23 bp, Sus scrofa
  source 1..23: mol_type = other DNA
ggaataattcatcattgccctgg

SEQ ID NO: 251 - DNA, 23 bp, Sus scrofa
  source 1..23: mol_type = other DNA
gccctggtcgctcccctccctgg

SEQ ID NO: 252 - DNA, 23 bp, Sus scrofa
  source 1..23: mol_type = other DNA
ccctggtcgctcccctccctggg

SEQ ID NO: 253 - DNA, 23 bp, Sus scrofa
  source 1..23: mol_type = other DNA
cccagggaggggagcgaccaggg

SEQ ID NO: 254 - DNA, 23 bp, Sus scrofa
  source 1..23: mol_type = other DNA
acccagggaggggagcgaccagg

SEQ ID NO: 255 - DNA, 23 bp, Sus scrofa
  source 1..23: mol_type = other DNA
tctgactgggacccagggagggg

SEQ ID NO: 256 - DNA, 23 bp, Sus scrofa
  source 1..23: mol_type = other DNA
ttctgactgggacccagggaggg

SEQ ID NO: 257 - DNA, 23 bp, Sus scrofa
  source 1..23: mol_type = other DNA
cttctgactgggacccagggagg

SEQ ID NO: 258 - DNA, 23 bp, Sus scrofa
  source 1..23: mol_type = other DNA
ctccctgggtcccagtcagaagg

SEQ ID NO: 259 - DNA, 23 bp, Sus scrofa
  source 1..23: mol_type = other DNA
tccctgggtcccagtcagaaggg

SEQ ID NO: 260 - DNA, 23 bp, Sus scrofa
  source 1..23: mol_type = other DNA
gcccttctgactgggacccaggg

SEQ ID NO: 261 - DNA, 23 bp, Sus scrofa
  source 1..23: mol_type = other DNA
ggcccttctgactgggacccagg

SEQ ID NO: 262 - DNA, 23 bp, Sus scrofa
  source 1..23: mol_type = other DNA
tgcgcagggcccttctgactggg

SEQ ID NO: 263 - DNA, 23 bp, Sus scrofa
  source 1..23: mol_type = other DNA
ctgcgcagggcccttctgactgg

SEQ ID NO: 264 - DNA, 23 bp, Sus scrofa
  source 1..23: mol_type = other DNA
cagtcagaagggccctgcgcagg

SEQ ID NO: 265 - DNA, 23 bp, Sus scrofa
  source 1..23: mol_type = other DNA
ggccctgcgcaggatgcatctgg

SEQ ID NO: 266 - DNA, 23 bp, Sus scrofa
  source 1..23: mol_type = other DNA
gccctgcgcaggatgcatctggg

SEQ ID NO: 267 - DNA, 23 bp, Sus scrofa
  source 1..23: mol_type = other DNA
ccctgcgcaggatgcatctgggg

SEQ ID NO: 268 - DNA, 23 bp, Sus scrofa
  source 1..23: mol_type = other DNA
ccccagatgcatcctgcgcaggg

SEQ ID NO: 269 - DNA, 23 bp, Sus scrofa
  source 1..23: mol_type = other DNA
gccccagatgcatcctgcgcagg

SEQ ID NO: 270 - DNA, 23 bp, Sus scrofa
  source 1..23: mol_type = other DNA
aggatgcatctggggctctaagg

SEQ ID NO: 271 - DNA, 23 bp, Sus scrofa
  source 1..23: mol_type = other DNA
ggatgcatctggggctctaaggg

SEQ ID NO: 272 - DNA, 23 bp, Sus scrofa
  source 1..23: mol_type = other DNA
tcaggctctccccgcaggctggg

SEQ ID NO: 273 - DNA, 23 bp, Sus scrofa
  source 1..23: mol_type = other DNA
ttcaggctctccccgcaggctgg

SEQ ID NO: 274 - DNA, 23 bp, Sus scrofa
  source 1..23: mol_type = other DNA
agcctgcggggagagcctgaagg

SEQ ID NO: 275 - DNA, 23 bp, Sus scrofa
  source 1..23: mol_type = other DNA
ggccttcaggctctccccgcagg

SEQ ID NO: 276 - DNA, 23 bp, Sus scrofa
  source 1..23: mol_type = other DNA
gggagagcctgaaggcccctcgg

SEQ ID NO: 277 - DNA, 23 bp, Sus scrofa
  source 1..23: mol_type = other DNA
ggagagcctgaaggcccctcggg

SEQ ID NO: 278 - DNA, 23 bp, Sus scrofa
  source 1..23: mol_type = other DNA
gagcctgaaggcccctcgggtgg

SEQ ID NO: 279 - DNA, 23 bp, Sus scrofa
  source 1..23: mol_type = other DNA
cctgaaggcccctcgggtggtgg

SEQ ID NO: 280 - DNA, 23 bp, Sus scrofa
  source 1..23: mol_type = other DNA
ccaccacccgaggggccttcagg

SEQ ID NO: 281 - DNA, 23 bp, Sus scrofa
  source 1..23: mol_type = other DNA
ctgaaggcccctcgggtggtggg

SEQ ID NO: 282 - DNA, 23 bp, Sus scrofa
  source 1..23: mol_type = other DNA
aaggcccctcgggtggtgggcgg

SEQ ID NO: 283 - DNA, 23 bp, Sus scrofa
  source 1..23: mol_type = other DNA
aggcccctcgggtggtgggcggg

SEQ ID NO: 284 - DNA, 23 bp, Sus scrofa
  source 1..23: mol_type = other DNA
ggcccctcgggtggtgggcgggg

SEQ ID NO: 285 - DNA, 23 bp, Sus scrofa
  source 1..23: mol_type = other DNA
ctccccgcccaccacccgagggg

SEQ ID NO: 286 - DNA, 23 bp, Sus scrofa
  source 1..23: mol_type = other DNA
tctccccgcccaccacccgaggg

SEQ ID NO: 287 - DNA, 23 bp, Sus scrofa
  source 1..23: mol_type = other DNA
ttctccccgcccaccacccgagg

SEQ ID NO: 288 - DNA, 23 bp, Sus scrofa
  source 1..23: mol_type = other DNA
tcgggtggtgggcggggagaagg

SEQ ID NO: 289 - DNA, 23 bp, Sus scrofa
  source 1..23: mol_type = other DNA
gggcggggagaaggcctctgtgg

SEQ ID NO: 290 - DNA, 23 bp, Sus scrofa
  source 1..23: mol_type = other DNA
agaaggcctctgtggattcctgg

SEQ ID NO: 291 - DNA, 23 bp, Sus scrofa
  source 1..23: mol_type = other DNA
cctctgtggattcctggccctgg

SEQ ID NO: 292 - DNA, 23 bp, Sus scrofa
  source 1..23: mol_type = other DNA
ccagggccaggaatccacagagg

SEQ ID NO: 293 - DNA, 23 bp, Sus scrofa
  source 1..23: mol_type = other DNA
tgtggattcctggccctggcagg

SEQ ID NO: 294 - DNA, 23 bp, Sus scrofa
  source 1..23: mol_type = other DNA
gatgctgacctgccagggccagg

SEQ ID NO: 295 - DNA, 23 bp, Sus scrofa
  source 1..23: mol_type = other DNA
tactggatgctgacctgccaggg

SEQ ID NO: 296 - DNA, 23 bp, Sus scrofa
  source 1..23: mol_type = other DNA
gtactggatgctgacctgccagg

SEQ ID NO: 297 - DNA, 23 bp, Sus scrofa
  source 1..23: mol_type = other DNA
agatgtgctgtttgttgtactgg

SEQ ID NO: 298 - DNA, 23 bp, Sus scrofa
  source 1..23: mol_type = other DNA
tacaacaaacagcacatctgtgg

SEQ ID NO: 299 - DNA, 23 bp, Sus scrofa
  source 1..23: mol_type = other DNA
aacaaacagcacatctgtggagg

SEQ ID NO: 300 - DNA, 23 bp, Sus scrofa
  source 1..23: mol_type = other DNA
acaaacagcacatctgtggaggg

SEQ ID NO: 301 - DNA, 23 bp, Sus scrofa
  source 1..23: mol_type = other DNA
catctgtggagggagcatcctgg

SEQ ID NO: 302 - DNA, 23 bp, Sus scrofa
  source 1..23: mol_type = other DNA
ggagcatcctggacccccactgg

SEQ ID NO: 303 - DNA, 23 bp, Sus scrofa
  source 1..23: mol_type = other DNA
tgaggatccagtgggggtccagg

SEQ ID NO: 304 - DNA, 23 bp, Sus scrofa
  source 1..23: mol_type = other DNA
ggacccccactggatcctcacgg

SEQ ID NO: 305 - DNA, 23 bp, Sus scrofa
  source 1..23: mol_type = other DNA
ctgccgtgaggatccagtggggg

SEQ ID NO: 306 - DNA, 23 bp, Sus scrofa
  source 1..23: mol_type = other DNA
gctgccgtgaggatccagtgggg

SEQ ID NO: 307 - DNA, 23 bp, Sus scrofa
  source 1..23: mol_type = other DNA
ggctgccgtgaggatccagtggg

SEQ ID NO: 308 - DNA, 23 bp, Sus scrofa
  source 1..23: mol_type = other DNA
gggctgccgtgaggatccagtgg

SEQ ID NO: 309 - DNA, 23 bp, Sus scrofa
  source 1..23: mol_type = other DNA
agaagcagtgggctgccgtgagg

SEQ ID NO: 310 - DNA, 23 bp, Sus scrofa
  source 1..23: mol_type = other DNA
tcacggcagcccactgcttctgg

SEQ ID NO: 311 - DNA, 23 bp, Sus scrofa
  source 1..23: mol_type = other DNA
gcagcccactgcttctggtaagg

SEQ ID NO: 312 - DNA, 23 bp, Sus scrofa
  source 1..23: mol_type = other DNA
tgggccttaccagaagcagtggg

SEQ ID NO: 313 - DNA, 23 bp, Sus scrofa
  source 1..23: mol_type = other DNA
gtgggccttaccagaagcagtgg

SEQ ID NO: 314 - DNA, 23 bp, Sus scrofa
  source 1..23: mol_type = other DNA
ctgcttctggtaaggcccaccgg

SEQ ID NO: 315 - DNA, 23 bp, Sus scrofa
  source 1..23: mol_type = other DNA
tgcttctggtaaggcccaccggg

SEQ ID NO: 316 - DNA, 23 bp, Sus scrofa
  source 1..23: mol_type = other DNA
gcttctggtaaggcccaccgggg

SEQ ID NO: 317 - DNA, 23 bp, Sus scrofa
  source 1..23: mol_type = other DNA
tctggtaaggcccaccggggagg

SEQ ID NO: 318 - DNA, 23 bp, Sus scrofa
  source 1..23: mol_type = other DNA
ggtaaggcccaccggggaggagg

SEQ ID NO: 319 - DNA, 23 bp, Sus scrofa
  source 1..23: mol_type = other DNA
agcatctcgatgtgcccaactgg

SEQ ID NO: 320 - DNA, 23 bp, Sus scrofa
  source 1..23: mol_type = other DNA
tctcgatgtgcccaactggaagg

SEQ ID NO: 321 - DNA, 23 bp, Sus scrofa
  source 1..23: mol_type = other DNA
atgtgcccaactggaaggtgagg

SEQ ID NO: 322 - DNA, 23 bp, Sus scrofa
  source 1..23: mol_type = other DNA
tgtgcccaactggaaggtgaggg

SEQ ID NO: 323 - DNA, 23 bp, Sus scrofa
  source 1..23: mol_type = other DNA
cccaactggaaggtgagggccgg

SEQ ID NO: 324 - DNA, 23 bp, Sus scrofa
  source 1..23: mol_type = other DNA
ccggccctcaccttccagttggg

SEQ ID NO: 325 - DNA, 23 bp, Sus scrofa
  source 1..23: mol_type = other DNA
gccggccctcaccttccagttgg

SEQ ID NO: 326 - DNA, 23 bp, Sus scrofa
  source 1..23: mol_type = other DNA
gagggccggctcagacaaactgg

SEQ ID NO: 327 - DNA, 23 bp, Sus scrofa
  source 1..23: mol_type = other DNA
agggccggctcagacaaactggg

SEQ ID NO: 328 - DNA, 23 bp, Sus scrofa
  source 1..23: mol_type = other DNA
gttgcccagtttgtctgagccgg

SEQ ID NO: 329 - DNA, 23 bp, Sus scrofa
  source 1..23: mol_type = other DNA
caacttcccatccctgcctgtgg

SEQ ID NO: 330 - DNA, 23 bp, Sus scrofa
  source 1..23: mol_type = other DNA
tcttggccacaggcagggatggg

SEQ ID NO: 331 - DNA, 23 bp, Sus scrofa
  source 1..23: mol_type = other DNA
atcttggccacaggcagggatgg

SEQ ID NO: 332 - DNA, 23 bp, Sus scrofa
  source 1..23: mol_type = other DNA
gaagatcttggccacaggcaggg

SEQ ID NO: 333 - DNA, 23 bp, Sus scrofa
  source 1..23: mol_type = other DNA
tgaagatcttggccacaggcagg

SEQ ID NO: 334 - DNA, 23 bp, Sus scrofa
  source 1..23: mol_type = other DNA
gtgatgaagatcttggccacagg

SEQ ID NO: 335 - DNA, 23 bp, Sus scrofa
  source 1..23: mol_type = other DNA
gggctcagtgatgaagatcttgg

SEQ ID NO: 336 - DNA, 23 bp, Sus scrofa
  source 1..23: mol_type = other DNA
tctttgggggacgtgctgttggg

SEQ ID NO: 337 - DNA, 23 bp, Sus scrofa
  source 1..23: mol_type = other DNA
ctctttgggggacgtgctgttgg

SEQ ID NO: 338 - DNA, 23 bp, Sus scrofa
  source 1..23: mol_type = other DNA
ggcaatgtctttctctttggggg

SEQ ID NO: 339 - DNA, 23 bp, Sus scrofa
  source 1..23: mol_type = other DNA
gggcaatgtctttctctttgggg

SEQ ID NO: 340 - DNA, 23 bp, Sus scrofa
  source 1..23: mol_type = other DNA
agggcaatgtctttctctttggg

SEQ ID NO: 341 - DNA, 23 bp, Sus scrofa
  source 1..23: mol_type = other DNA
aagggcaatgtctttctctttgg

SEQ ID NO: 342 - DNA, 23 bp, Sus scrofa
  source 1..23: mol_type = other DNA
cgggagctgcagcttcacaaggg

SEQ ID NO: 343 - DNA, 23 bp, Sus scrofa
  source 1..23: mol_type = other DNA
gcgggagctgcagcttcacaagg

SEQ ID NO: 344 - DNA, 23 bp, Sus scrofa
  source 1..23: mol_type = other DNA
cagctcccgctcacgttctccgg

SEQ ID NO: 345 - DNA, 23 bp, Sus scrofa
  source 1..23: mol_type = other DNA
tctcaccggagaacgtgagcggg

SEQ ID NO: 346 - DNA, 23 bp, Sus scrofa
  source 1..23: mol_type = other DNA
ctctcaccggagaacgtgagcgg

SEQ ID NO: 347 - DNA, 23 bp, Sus scrofa
  source 1..23: mol_type = other DNA
tcacgttctccggtgagagacgg

SEQ ID NO: 348 - DNA, 23 bp, Sus scrofa
  source 1..23: mol_type = other DNA
gcagggaggccgtctctcaccgg

SEQ ID NO: 349 - DNA, 23 bp, Sus scrofa
  source 1..23: mol_type = other DNA
gagacggcctccctgccgtcagg

SEQ ID NO: 350 - DNA, 23 bp, Sus scrofa
  source 1..23: mol_type = other DNA
agacggcctccctgccgtcaggg

SEQ ID NO: 351 - DNA, 23 bp, Sus scrofa
  source 1..23: mol_type = other DNA
cggcctccctgccgtcagggagg

SEQ ID NO: 352 - DNA, 23 bp, Sus scrofa
  source 1..23: mol_type = other DNA
ggcctccctgccgtcagggaggg

SEQ ID NO: 353 - DNA, 23 bp, Sus scrofa
  source 1..23: mol_type = other DNA
agccctccctgacggcagggagg

SEQ ID NO: 354 - DNA, 23 bp, Sus scrofa
  source 1..23: mol_type = other DNA
ctgagccctccctgacggcaggg

SEQ ID NO: 355 - DNA, 23 bp, Sus scrofa
  source 1..23: mol_type = other DNA
cctgccgtcagggagggctcagg

SEQ ID NO: 356 - DNA, 23 bp, Sus scrofa
  source 1..23: mol_type = other DNA
cctgagccctccctgacggcagg

SEQ ID NO: 357 - DNA, 23 bp, Sus scrofa
  source 1..23: mol_type = other DNA
agggcctgagccctccctgacgg

SEQ ID NO: 358 - DNA, 23 bp, Sus scrofa
  source 1..23: mol_type = other DNA
gtcagggagggctcaggccctgg

SEQ ID NO: 359 - DNA, 23 bp, Sus scrofa
  source 1..23: mol_type = other DNA
tcagggagggctcaggccctggg

SEQ ID NO: 360 - DNA, 23 bp, Sus scrofa
  source 1..23: mol_type = other DNA
ttgaaaagtggatatgcccaggg

SEQ ID NO: 361 - DNA, 23 bp, Sus scrofa
  source 1..23: mol_type = other DNA
cttgaaaagtggatatgcccagg

SEQ ID NO: 362 - DNA, 23 bp, Sus scrofa
  source 1..23: mol_type = other DNA
caaactgaggacttgaaaagtgg

SEQ ID NO: 363 - DNA, 23 bp, Sus scrofa
  source 1..23: mol_type = other DNA
tggaaacagggggcaaactgagg

SEQ ID NO: 364 - DNA, 23 bp, Sus scrofa
  source 1..23: mol_type = other DNA
acatatttgctggaaacaggggg

SEQ ID NO: 365 - DNA, 23 bp, Sus scrofa
  source 1..23: mol_type = other DNA
gacatatttgctggaaacagggg

SEQ ID NO: 366 - DNA, 23 bp, Sus scrofa
  source 1..23: mol_type = other DNA
tgacatatttgctggaaacaggg

SEQ ID NO: 367 - DNA, 23 bp, Sus scrofa
  source 1..23: mol_type = other DNA
ctgacatatttgctggaaacagg

SEQ ID NO: 368 - DNA, 23 bp, Sus scrofa
  source 1..23: mol_type = other DNA
tgtgaggctgacatatttgctgg

SEQ ID NO: 369 - DNA, 23 bp, Sus scrofa
  source 1..23: mol_type = other DNA
tgtcagcctcacatcactgaagg

SEQ ID NO: 370 - DNA, 23 bp, Sus scrofa
  source 1..23: mol_type = other DNA
agcctcacatcactgaaggcagg

SEQ ID NO: 371 - DNA, 23 bp, Sus scrofa
  source 1..23: mol_type = other DNA
ttcctgccttcagtgatgtgagg

SEQ ID NO: 372 - DNA, 23 bp, Sus scrofa
  source 1..23: mol_type = other DNA
gggtcagctgacagcttgagggg

SEQ ID NO: 373 - DNA, 23 bp, Sus scrofa
  source 1..23: mol_type = other DNA
ggggtcagctgacagcttgaggg

SEQ ID NO: 374 - DNA, 23 bp, Sus scrofa
  source 1..23: mol_type = other DNA
gggggtcagctgacagcttgagg

SEQ ID NO: 375 - DNA, 23 bp, Sus scrofa
  source 1..23: mol_type = other DNA
tcagctgaccccccatcctgagg

SEQ ID NO: 376 - DNA, 23 bp, Sus scrofa
  source 1..23: mol_type = other DNA
aaaacagacctcaggatgggggg

SEQ ID NO: 377 - DNA, 23 bp, Sus scrofa
  source 1..23: mol_type = other DNA
gaaaacagacctcaggatggggg

SEQ ID NO: 378 - DNA, 23 bp, Sus scrofa
  source 1..23: mol_type = other DNA
ggaaaacagacctcaggatgggg

SEQ ID NO: 379 - DNA, 23 bp, Sus scrofa
  source 1..23: mol_type = other DNA
gggaaaacagacctcaggatggg

SEQ ID NO: 380 - DNA, 23 bp, Sus scrofa
  source 1..23: mol_type = other DNA
ggggaaaacagacctcaggatgg

SEQ ID NO: 381 - DNA, 23 bp, Sus scrofa
  source 1..23: mol_type = other DNA
acgtggggaaaacagacctcagg

SEQ ID NO: 382 - DNA, 23 bp, Sus scrofa
  source 1..23: mol_type = other DNA
tgaggtctgttttccccacgtgg

SEQ ID NO: 383 - DNA, 23 bp, Sus scrofa
  source 1..23: mol_type = other DNA
tttccccacgtggagcccctcgg

SEQ ID NO: 384 - DNA, 23 bp, Sus scrofa
  source 1..23: mol_type = other DNA
ttccccacgtggagcccctcggg

SEQ ID NO: 385 - DNA, 23 bp, Sus scrofa
  source 1..23: mol_type = other DNA
cacccgaggggctccacgtgggg

SEQ ID NO: 386 - DNA, 23 bp, Sus scrofa
  source 1..23: mol_type = other DNA
tcacccgaggggctccacgtggg

SEQ ID NO: 387 - DNA, 23 bp, Sus scrofa
  source 1..23: mol_type = other DNA
ctcacccgaggggctccacgtgg

SEQ ID NO: 388 - DNA, 23 bp, Sus scrofa
  source 1..23: mol_type = other DNA
atctgtgattctcacccgagggg

SEQ ID NO: 389 - DNA, 23 bp, Sus scrofa
  source 1..23: mol_type = other DNA
gatctgtgattctcacccgaggg

SEQ ID NO: 390 - DNA, 23 bp, Sus scrofa
  source 1..23: mol_type = other DNA
tgatctgtgattctcacccgagg

SEQ ID NO: 391 - DNA, 23 bp, Sus scrofa
  source 1..23: mol_type = other DNA
aatcacagatcacccattcctgg

SEQ ID NO: 392 - DNA, 23 bp, Sus scrofa
  source 1..23: mol_type = other DNA
acccattcctggagtttgtgagg

SEQ ID NO: 393 - DNA, 23 bp, Sus scrofa
  source 1..23: mol_type = other DNA
cccattcctggagtttgtgaggg

SEQ ID NO: 394 - DNA, 23 bp, Sus scrofa
  source 1..23: mol_type = other DNA
ccctcacaaactccaggaatggg

SEQ ID NO: 395 - DNA, 23 bp, Sus scrofa
  source 1..23: mol_type = other DNA
tccctcacaaactccaggaatgg

SEQ ID NO: 396 - DNA, 23 bp, Sus scrofa
  source 1..23: mol_type = other DNA
tcaggtccctcacaaactccagg

SEQ ID NO: 397 - DNA, 23 bp, Sus scrofa
  source 1..23: mol_type = other DNA
tttgtgagggacctgaaagatgg

SEQ ID NO: 398 - DNA, 23 bp, Sus scrofa
  source 1..23: mol_type = other DNA
gatgggtcagaccatctttcagg

SEQ ID NO: 399 - DNA, 25 bp, Sus scrofa
  source 1..25: mol_type = other DNA
gggcaatgatgaattattccaggag

SEQ ID NO: 400 - DNA, 25 bp, Sus scrofa
  source 1..25: mol_type = other DNA
tctgactgggacccagggaggggag

SEQ ID NO: 401 - DNA, 25 bp, Sus scrofa
  source 1..25: mol_type = other DNA
cttctgactgggacccagggagggg

SEQ ID NO: 402 - DNA, 25 bp, Sus scrofa
  source 1..25: mol_type = other DNA
gcccttctgactgggacccagggag

SEQ ID NO: 403 - DNA, 25 bp, Sus scrofa
  source 1..25: mol_type = other DNA
ggccctgcgcaggatgcatctgggg

SEQ ID NO: 404 - DNA, 25 bp, Sus scrofa
  source 1..25: mol_type = other DNA
ggagagcctgaaggcccctcgggtg

SEQ ID NO: 405 - DNA, 25 bp, Sus scrofa
  source 1..25: mol_type = other DNA
gagcctgaaggcccctcgggtggtg

SEQ ID NO: 406 - DNA, 25 bp, Sus scrofa
  source 1..25: mol_type = other DNA
ctgaaggcccctcgggtggtgggcg

SEQ ID NO: 407 - DNA, 25 bp, Sus scrofa
  source 1..25: mol_type = other DNA
aaggcccctcgggtggtgggcgggg

SEQ ID NO: 408 - DNA, 25 bp, Sus scrofa
  source 1..25: mol_type = other DNA
ggcccctcgggtggtgggcggggag

SEQ ID NO: 409 - DNA, 25 bp, Sus scrofa
  source 1..25: mol_type = other DNA
ttctccccgcccaccacccgagggg

SEQ ID NO: 410 - DNA, 25 bp, Sus scrofa
  source 1..25: mol_type = other DNA
tacaacaaacagcacatctgtggag

SEQ ID NO: 411 - DNA, 25 bp, Sus scrofa
  source 1..25: mol_type = other DNA
acaaacagcacatctgtggagggag

SEQ ID NO: 412 - DNA, 25 bp, Sus scrofa
  source 1..25: mol_type = other DNA
ggctgccgtgaggatccagtggggg

SEQ ID NO: 413 - DNA, 25 bp, Sus scrofa
  source 1..25: mol_type = other DNA
gggctgccgtgaggatccagtgggg

SEQ ID NO: 414 - DNA, 25 bp, Sus scrofa
  source 1..25: mol_type = other DNA
ctgcttctggtaaggcccaccgggg

SEQ ID NO: 415 - DNA, 25 bp, Sus scrofa
  source 1..25: mol_type = other DNA
gcttctggtaaggcccaccggggag

SEQ ID NO: 416 - DNA, 25 bp, Sus scrofa
  source 1..25: mol_type = other DNA
tctggtaaggcccaccggggaggag

SEQ ID NO: 417 - DNA, 25 bp, Sus scrofa
  source 1..25: mol_type = other DNA
tctcgatgtgcccaactggaaggtg

SEQ ID NO: 418 - DNA, 25 bp, Sus scrofa
  source 1..25: mol_type = other DNA
agggcaatgtctttctctttggggg

SEQ ID NO: 419 - DNA, 25 bp, Sus scrofa
  source 1..25: mol_type = other DNA
aagggcaatgtctttctctttgggg

SEQ ID NO: 420 - DNA, 25 bp, Sus scrofa
  source 1..25: mol_type = other DNA
cagctcccgctcacgttctccggtg

SEQ ID NO: 421 - DNA, 25 bp, Sus scrofa
  source 1..25: mol_type = other DNA
tctcaccggagaacgtgagcgggag

SEQ ID NO: 422 - DNA, 25 bp, Sus scrofa
  source 1..25: mol_type = other DNA
gcagggaggccgtctctcaccggag

SEQ ID NO: 423 - DNA, 25 bp, Sus scrofa
  source 1..25: mol_type = other DNA
agacggcctccctgccgtcagggag

SEQ ID NO: 424 - DNA, 25 bp, Sus scrofa
  source 1..25: mol_type = other DNA
ctgagccctccctgacggcagggag

SEQ ID NO: 425 - DNA, 25 bp, Sus scrofa
  source 1..25: mol_type = other DNA
tgacatatttgctggaaacaggggg

SEQ ID NO: 426 - DNA, 25 bp, Sus scrofa
  source 1..25: mol_type = other DNA
ctgacatatttgctggaaacagggg

SEQ ID NO: 427 - DNA, 25 bp, Sus scrofa
  source 1..25: mol_type = other DNA
gggggtcagctgacagcttgagggg

SEQ ID NO: 428 - DNA, 25 bp, Sus scrofa
  source 1..25: mol_type = other DNA
ggaaaacagacctcaggatgggggg

SEQ ID NO: 429 - DNA, 25 bp, Sus scrofa
  source 1..25: mol_type = other DNA
gggaaaacagacctcaggatggggg

SEQ ID NO: 430 - DNA, 25 bp, Sus scrofa
  source 1..25: mol_type = other DNA
ggggaaaacagacctcaggatgggg

SEQ ID NO: 431 - DNA, 25 bp, Sus scrofa
  source 1..25: mol_type = other DNA
tgaggtctgttttccccacgtggag

SEQ ID NO: 432 - DNA, 25 bp, Sus scrofa
  source 1..25: mol_type = other DNA
ttccccacgtggagcccctcgggtg

SEQ ID NO: 433 - DNA, 25 bp, Sus scrofa
  source 1..25: mol_type = other DNA
ctcacccgaggggctccacgtgggg

SEQ ID NO: 434 - DNA, 25 bp, Sus scrofa
  source 1..25: mol_type = other DNA
tgatctgtgattctcacccgagggg

SEQ ID NO: 435 - DNA, 25 bp, Sus scrofa
  source 1..25: mol_type = other DNA
aatcacagatcacccattcctggag

SEQ ID NO: 436 - DNA, 25 bp, Sus scrofa
  source 1..25: mol_type = other DNA
ccctcacaaactccaggaatgggtg

SEQ ID NO: 437 - DNA, 41727 bp, Sus scrofa
  source 1..41727: mol_type = genomic DNA
ctttgaggattttccaagttttcttgcaaaatcagctgtaattttaaagggtattaacatttactctatggagaactttttgactgttttgagtgtgacattatttcagaatatatagtctgccacactgctgtgaacagaactccactgcttatttttaacatatgataatactgttttgttttcattatattacagctgctcctcttggttgatagttctgatttctgcttgtatttatggaagacagagtaaagggggtttaacttcagagctggttggagtcagtggttccatgatgttaccaagaattgggtttcttggagttccctagtggctcagcaggttaaggatctggcattgcccctactgtggcttaggtcgctgctgtggcgtgggtttggtccttggcccaggaacttctgcatgccatagcggtagcaaaaaaaaaaaaaaaaaaaatcgtgtttcttgtcgtctccatgctgtggttttggatggaagaactacttttatcatggtaccagctccggggtgacggagacggccaccaacaattcccgggactacttgttcttcttcctccaagcataaggaagagggtctctggaaattcctgcagaagagttaaaaaaaaaaaaaaagagagagtttccccaccccaactccaaaccccaggagacctctcttccaggtctctctggccaaaaatgggctgtgtgctcatccctgaagcaagaagtctggcagagaatgggattcggctgcagagcccggcccggggccacacccccacccctagcactggaggggagctgctcctgggagatgaggacaccccaacaaaagtgccagaaagctgccacgagaaagggacctgactaatgagaaagcaagcgcaagaggatgagggtagggacactcatactgcctggggtgtaggtggggaccctggtgtttgatggaacaatggcgccattaccagagggctctgtacacaccattccaaacagaaataccttgtctatctactggagaccgtccccgagcgtcttccacctggccaacctggacaccagggaaggaggagagatggaaggaccagggcataccagaggcggccccctgcggagggtggaagggcggaaaccaaaggagagggtgggaagaatttacaggcaggttcagctcccaggacacctgctctggaaggagcgctgagccaacccagcctgggatctgggatgaacagactttcttccacgtccacctgattctaaagcaatccatcttgatttgcgtattacaaacagcgttggaaagagcctctcaaggacgggcagaaagccatgccttctagacgagagttcatctcaccctcttccctccctggtctcttgccttaggtgggcgggaggacgtggagccgctcctggggacgttcccagggagagggaggggcttgaaacctgagggccgagaagggccacccgagcggattggggagggtccccaccccaagcgctgcgtcttgggctcgagggcccggccagcgggccctgggagcagacgcctgggtcctcgccgtgggcacaggggccatgcgagtcccaggtgccagggcccggccggctgatacgagacgcgatcagcgctccccagaatcccgctcctgaagcgccggcgacctgctcggtcccggggacgtgagagggagcctcgctcccccccccccccccggccccaaactgcccgccgggaacggaagcgcccccggagctccctctgggtcccgccgagagcgcgtcccgggttttcccgggggaaactcctccccccgcctcgccgccccgcccggacccgcctcgcccgggacggccgttcccgtccgtcccctccccctgcccgaggtgacctgggaccgcgcttggggtgtcaccctgggccctgggacaccgcctccggggattaaagccagagccggggaggatcggaactccgagctgggccgaggaagccgcgagcttcggaggaggcggaggagaaggaagagcgaggggcggggagcgcgggacccggagcgcggcaggtaagcggcgcgcgggccgggtgttcggtgccagggctggggccggggggcctgggctccgcggaggacctggtcgccggggagcgacctacccgctctcggcactaggcgggagtcacgggaagtgaggtcaggtgcgtgcgagcggctccagctcgacaggtgcgtcctctcgccggcggggcgcgcggggctgagccgggcggagcgacctccccgaggtcaggaggattccgcgcgcctgggaccccttggctcgctggtcagaggcccccgactgccccgttggcatcgctcgctggggcaggtggccttgtgagtccttagccaatcgatgggagaaacgggtgctggagatccccagagtgggtcccctccacctgccctctgcggccgagctgggaacaggggtgcgccttgtccctttggggagaggaagaaggaggggtgtccatctgcttctttgtttcgggaaaatctctgcagccccgtagtgttacctgaactcctgcttcttggttccctccccaagtggggagaaactggaaactcactaagatgccctgagcgatagggcccgcgaggcaggaacaggtccggctcagcagtccccggaggggactgcacgcagccggagtcccattgcagtgaccctgaggcgacgatcagagcagggcagcctgggcaccgggcacccatacacatccctgcttcacccccggaaggagcagtcctggccactagaccctcaatccctgccagagacaaggccaggcacttgggtgcttcggttcaccaaatgccattaggcaggatccaggaccatagctgaacacaccagcctggcaatgagcgccctgcctacggtttcgtgcgccctacagaagagttgcctcatggctccaaatttcgcgctcctatgccttagagggcctggttgcgggtggctgccagctttaccttgtgccccctgttttgcgtgcctggagctgagggcctgccccgccggagggcacagcgtggtgatcacgaagaccttcggggatacacaggaggcagctgggacaggtggcatcctagtgggtcgcttgggagggaaaggacagcgccccagttctgcctcttgggccggggtcaccccgggctctgcctcaagactctgaaacctgcctccattcaaacccgtgccctagtcctggagccataagcaaggtcagggcacagggggggactcgccgctgctcctggcactgctggcgctcacagctgagccaaccaaagaatggaaaattacaaaggctgctcagaggagttccaaggtgtttagaaaacctgaagtgaggggagtgttttcgacttagcctgggaggtcgtggaagcttctccaggaagtgaggaagaagccaaccttccttggaggagggtgggggctggggatgtgggcaggacttccagggaggcggggggcactccagccaaccttccttgacccatgaggtcagagagaggctagcccccccccccagcaagcactcagcacaggagtcaggggttgcggtgcgggcctcgcctggaaatagttttaaaaacatcagctggcaacagcagggacctttgttcttggtgtggtcgtttgtttggtctcctgcagtgtcattcttcttgacacctcctaggtcagtcttctttcaacaaggttgcgtagctgcccagagccacctgagagccaggagaaccctgggctgctccccccgccgggctcagtcgcctgttttggccacactgatgtgattgtcacagcaaaggggaacgtcctattttcttccagccgagcatccctccaagctcttatactgtatttaactccgtggcgagggaactgagacagagagcgcgggaccaactggctgccgtcccacagggagaacgggggtgggaggaaggggtgggggtgaaggcaggccctctggtccagggaggctgagaatgaggcctggaatcctcctgggaaaggcgcttgctcactctgcggccaggctggtttccccagaagccgggggcgccatgctgcccccttgcagagtgacccggcaggagagtgggtgctttgtcgggacgtgcagaaagctgacctccccaggcaagggggccgcgggaggcggggagcgtcctgtaggcgatgaagcctagacacacagggacattctatgggcagtaatcagcatcctgtgagcctgctgaccactcctctgggccctgcctgcagtgggggctccaccagcaggtgtgagtcaggcctgcgtgctggaggctcttactctttaacaccgaagtgtggggcaggagtcctgggggcttcgtaattgttggaggtcgttgcctgggtctcaattgccgcatcaggctctgagagaataggaaattctgtttttttcctaagccttccagaaaagtcctgtttggggaaccgggggtcaaactaagcctttcaggcatggaagtggagaggaaaggcgactgggtcagctgaggggctggccggctctgcttccctaaggagggaaggaaggccgacctgcaggtgcctgcaggtgcgcagcccaccgagtgggggaggggactccaggcctctcttccaacctctgcggctcccccagcaccgagtctctgggtaggcctgcttcccataactgccaagcctttgctgctcttcctggcccctcctgacttcccaggacacctgccagggcccccttgctgctgggtctgccccaggatgagtggggtggcctctggggatggggtgcaaaggtgtcacgctcctgacggctgagatctgaattgggccagccctggtgctcccttcctagctgggtgttggggagccctgcaggccttctcagggatccctggcttttgcaggcctctgaggtgtgggcctgggtgtgggggagggggactttgggggcccctctgtgcccttgaccaccctggtcccgaggacacatggagttgcaaggggctccgctgaccacaggggcagttctggggaccaatctagagacagagcttgaggtcaggaaggaagaggaacttggcttgccccagtcactggacagaggtcagaggaagaggaaccaggagcatgaaaaatgccacttgggccaccttagaggcatccaggggaaagggaggaccctgattttctggcgggggtgagagcggtgccttgggctgtaaactgtgtgggttttgaagagggaaggtgacgcaaagacgtgcttttggcttctgctagaggcgcatcgggaagaagagggaaggggtcctgtgggtggagaccccaggcattgcctttgcaccaggttgagccatcaggcatctgtaggggcaggggtgcgggagggggtcggggtgccgacatgtgctgagaaagcgggaaatggggcgttcccgttgtggctcagccgtaacaaacctgaccagcatccatgaggatgcaggttccatccctggcctctctcagtgggttaaggatccggtgttgctgggctgtggctgtggctgcggtgtcggccagcaggtgcagctccgattcgacccctagcccgggaacttctgtatgcttcaggtacaaccctaaaaagcaaaaaaagaaaagaaaggaaaagcaaagaaaaggaaagaaggcaggaaatgtttgaaagttgccagctgaaggtctggaagctcgtgcaagggcagagtagacagaaaagaacggaaaagaaaagagaaaagaaaggaaaagataagaaaaaagaaaaggagaaagcaggaactgtttaaaaggcaccagcatcattatacaactacagatgtgatgaattcatttgagtaataaaaaaaaaaatggaaaaaataaaaaataaaaggcaccagctgaagtctggaagctcgtgcagagtggacggaataattgttgaggcgctcagtcaggtcagcctgggaagaggggccatggtcatccagcagccattcagtagaaaactattttcttcttttttttttttgtcttttgtcttttttttgtctttttgaggaccacactcatggcatatggaggttcccaggctcggggtcgaatcagagctacagctatgccacagccacagcaacactggatccgagcctcgtcttggacctacaccacagctcacggcaacgctggatccttaacccactgagcgaggccaggggtcgaacctgcatcctcatgttcctagtcagatgaggttccgctgagccatgacgggaaccccattgagaactattttctgtgagctctgaactggcagatgaaactaagtccaggctttaggattgctcgtttgggtctgacccagtgtcaagccctttgaggcccttgtttttttttgggggggggcactcccagcagggcacacacttggcttgttttggcttgtttgagactcaatctgagcaccaggcacacaaaacccatgttttttagggatgggattggagtttatccttcgagtttctctctctcttttttttggggggggcaccctaaggtatatggatttcctgggccagggatcagatctgagccacacttgtgacctacactgaagcctcggcagcaccagctcctttaacccactgtgctgggctggggatggaacctgtgtcctggcaaagccgagacgccgccgatcccgttgtgccacagcgggaactcccacccttgagtttctaatcacagagaataaaagtcatcccgcctggcctcgcggctcaagccccatttggttctagaatgtgcttgagaaacgatcttcagaattggatcaaatgtttttttccggtttgaggcagtgtctcttctaaagcagcccaatccatgttgaaatttattgtgagaaactggtttcccggattgagctaaacctgccctcctgatatttctgaccattttgctccgggattcatcctgtgggtttacactgaatgaacggagtcccccctccacgccacagtcccttaatgctctgttatgttgtcccagggctcatcaggtgccaagatgcacacacatctgatcctgccccaaaccgtatttacttgttatgcgcgtttcacagactgacggggcaagcccttcaaactgtggccttcacacagggtggaggacgggttcctggagggacccggcacctctctggcccgcctgtcacatgcagttggctcggtgcgcctggctctgtgaggtcccccaggacctcggtttgggggctgctcttctctgctctctcgcttacgtttcatcttactcacttggcctctgggatgccagaaattaccggaacactttctttgtgcgctccaaagctagatttttttttaaaaaagggatcagacccatgggttgccctaaaaagtttgtaatcttgtttagacaatgaggcattcgtagaaaaagaaacttggacgatgcgggcagtacctgtcctgagctggtgtctctctctccgtctgtctctgtctgtctcagtgtctctctttctcattttggggtcagttaccgtggatgccatttctcttcttatcttccatttgtttatgtcgcactattacttaaatccacccctgtttgttaactttcctctgagagttcatttttccccagcaaggcacggagcatctttccatgtaatgcctaagatgtaattaggtgtatggggttttatgcggcggctgaggcccacagggatccatggatcgtggctcactggatggggagtgtccagcattgcttgctttggactttgaagaagggttaccgcatctgaggtccaggaacctggggctccttccacccatcccacctcatcccaccacctgtctgcgcctggaaggcacctgaccaggcccagggcaggagcgtgaagggtgtgttggggcaagtcaggggagccttcgttatggggcggcccctgacccctagaaggggtggggggtggcatctctgagtctgacctcacttgcattggtttgttggttttcaaaataagtcttcagcaggtgcacgtggaggtccagaggggagactcttcttcttgggaaatagcttcattaaatgggatgcaggtaggaacagttttcacaccttgggatgcaggtaggattccagggctggatcccgaagagcagcggccaggcaggtgactcggggtgtgagtcagcctctgaaaggagagctcagagaggcctgagatggccgtgtcctgtcctgtgaccatggcggggcgggacctcgccgggaacaaagagggggcggagtttgggcacgtgtgtggcttggctctttgtcacttcgaggaagagcccttcctcaaggctggctttgtggaaccaagggccatcctccacgtcctttaactgacaggaaagaacactcgagaagccgcatcgtgtcccgccagctaggctgtctcgctcctttcagaagccagtgggagagggttgctgtcttgcctgggcctggaattgcctgtttgagatgaaccgcagcagggaatgtgatctcctggtctctccagttgtctcccatcatcacatacactccccggggagacctctctttctttctctttggccacagagagctgtaggcagcatactcttgtcttctctcccaaaggccatggcagcggatctgggtgcacttggcattctggctccagggttctgtgggtcgtgggagcgctggtgagataggatcgtgtggtggttacgtctgggtcccgggcttttccaggccaatccccggccacgcggccccccgcacccctggcttcctggtctctcgctgttcggggtcccgaatgagtaggaagcatgggtttcgggcgttgatgcctcactcctttcagacggctcgtaaaccgggaaagccgccagtgggatcgaatgttcatttcctgaaacttcaaccaaacctctaactggggagatggcgcgaagccacagagggcagagaaaccagagagacgagactgttccctggggcaggagtcccctgagactaatctgaagaaggcacagagcttttcctgccgagaccagaggattttatgctgctgtcaggtccggatccctctctctttacagagcagcggtaggaaaggttattaaattgcgataatcacacccactcaaaggcttaaatgttgtgtgctttcagaacgccgctcccttgctgctgtgtgctgtcagttttcttccctcgtacccggaaaggggttgactctgtttttttttttttaatcttaagaaaaaatctcaaagctatcagtacaaactggttcttttattttatctctttacatgaagggtaaatacacattgggtgtgtttttcttgtgacctgttccacgaggtttcccaagattaagcagccatacttgtgtttacagcaaattcgtgtttgcgttttttttttttaagtgtaatgcatcaaataataattttgcttggtggtcagagaggcggcgccaggagggagctggctgtgcggcatttggccccattgggttttcagagctggtggatttcgtgttagtctcttgaggacacttttctgatacacatctgcaaatgctgccaatcgggcccatcctgcttctgggggctgttcaccatttatcagctttatgatcacttaaaaaattctttcgtcttggggtatagttgattgacaatgtattagattcaggtgcacagcccagtgaatcagtttcacgtttacacacacatacccattcttttctcccttgtaggttattacagaaccttgggtagccatccctgtgctgtacagtagggccttgttcgtatctattttatatatatgaccattttattaacaaaaacgtatgcagcgcttcctgcgtgccaggcactaagaacctcaccattgcctcgtttcagtcccccagcagctctgtagcgggtggtactgttactcctttccctgcatccttcccttgcagacaagaaaaccagggcctggacatgggaaccgatttgcccctggtcacagctagagcgtgactgagctggggcttgcacccacggtcgcgtcgcagaggtcatgggcttagcagctgtgctgtgctgcctgctgtgttcgagcccctgcctcgtgccaggcactgaacagatgaaagctgttccgtcctgatctggtcaggtcgcagcccagatgggtccagatcgaagctttcgtctggggacaccagccttcgcatggatgcgtgtaaaggccgtttctcgaagcattgtgtggttatctcaaggcagccaggagcagtggaaaaagccctcgtttgctcaggatggggagagtcatgtagaatcaacttttcacgtggccagtctttctagatgttacggttaactgtgcacacgtgtcagaaaacaaggcgacccagagcttccgcgtcattactttagaaaccccagcgaggtggtgaatttctttctctcctggatctgagggagcgagcggcatggctggggggtaagcatgtggtttaaggaatttttaacttatgtgtttgtgaagatgggggcggttccatgtggcttatttggaaagtccggtgtggctgccttcgggtactaagctctagggcacctgctccaccagggagaggtggggtgggctgctgcccaaggagtctcaccgcacacacactcccgcacctgccaccgggcccgtgaggtgtctgcctgtgtctggcgttaaatcctctcctgaacaagcccatgtgatgcctgcatctgctgctgcacgagactcggcgaggccacttgtctggtgacagctcaggacatggcattgccaggagctgaccccaggctttgccctccttcctcggggtccctgtgcccgtttctctttgcgtttggtgaagatgacgttgggaaagtctggtcctcctggtgggtgctgggtgggtgcgggaagccctgaactcaagctgcaggcaagcggggcccaggctcctttgggactagcggctgcaggaaaagacgctgcagcgaaggctaaggggcacgggcagagccgccctggttcccgagaaaagtggccctcacccagagagccttctggttctttcttgattcatcttctgccagctcaccggtatggaaattttacagtcttgtcattctttcttttaggtcaacttgaaagttccaaatacttacctgtacctgatgctgctgataaaatcaggatggctttaaactcagtaggtggttaattattacctccctggcctttcctctgtcccctgtgccttctgttagttccagcggtacctgttggtcagcattttaggagccaccatggggaacgcatgtgtcctgtgggtgctgcgaaggtgttcctggggttaggggtgagcaggagcggggcggagggagccccatgacaggtgcacggctgggagtctcggagcggtgggtggtgtgggtgatgggcttcttccctgggctgcttctcttcctgctccaagtgggggtggggcaggggacaggagcgggtgcggtgacagatagaggcaggatggtggtacaagggagtgagacctggggggaaggagggcgggatggaccaccagctggcagagcacagcggggcctcgcttcctggtaggctgggaaaggtggtgacagaatgggacactgggtctatccctgaactgccccccacccccaccccccgcctcacacgcagacaaatgtctttattccccctggcaccttaccgatcgtcttatacctgggaataagtgcctttagcaaagaactgtagttcataacaaaaatttcctctctcctctttctggaaaatagaccttggcacccccgcccccacccctttattgggtagtacttattttcactcaatcttcttaaaatttcagggagttccatggtggctcagcaggttgagactctgactggtatctatgaggatgtgggttcgatcccaggcctcgcgcagtgggttatggatctggcgttgccatgagctatagcgtaggttgcagatgaggcttgaagctggcgttgctgtggctgtggtgtaggctgtagctctgaatcgacccctggcctgggaccttccataggctgcgggtgcggccctaaaaaagacaaaagtaaaaaaataaaataaaatttcaagtctccggaacaggaattcacagggacaaactggcgctcttgccccagctctcctggggaggacagcctgttcctagatgctgcagatacaggccagaggccggccgtgcctcttttttttttgcctctggttccccatttagattagagattacatgttgttttttccttctgtgaccggtctggtttagagtgtaatctttggacttttgaaagagccgttggttggtgagttattaaaactcgccctgggctcattgctttctaatcgcgctgccctgggggccccgctgaaaattcggagcaccgaattgcttctgctcaggatgctggtcctccctgggctctcctcaacccagagcagcggagaaatgactcagtgggaacacatttgcccggaggatgcacagataagagggcaaagcagcgcaaggtcgcaggctgctggggcctggggggaggggggctgggggggccgtggtgtcagaaggtgggctcctgagggccaggccccagcgggcaattcttaagtaggcagagggggaggggaaggcattctaagacagaaaccagccgaaggaaggatggagctgagagcgagatgctgtggcagggtgctccttccacctttgggtgtgtgttaacgctgtgccccagaaattccattcgctgggacaggctgcagaaggcacacgctgagcactaaggcccgtgctggacaaagctgctcatggcagcacatctgccagcggctccctcagtggcagatcctttaggtcgattttgggttttttttgctttttttagggctgcacctatggcatatggaggttcccaggctaagggtcaaaccagagctaagctgctggcctgcgccacagccacagcaatgcaggatctaaaccgcgtccttgacctacaccacacagctcatggcaatgccggatccttaacccattgagtgaggcctcggggatcgaacctgcatcctcgtggatgctggttagaatcgttaatcactgaaccaccaagggaacgccttgatctcttcatctttgtcccttatgttcctgtctgcaaaatgggtctatcggtcatagctggagcatcttaggggtgagggtcaggcctcagagagggcaggggtacagaccctcttgcagaggagttcaccaatgtcaggcgttattttttctccttgtaattctggtacccttgttccttactccaagcccattctcaggggatggggattggctcattcccagacttctttttcctttttttttttctttttagggccgtacccacagcatacggaggttcccaggctaggggtccaatcagaactgcagctgccagcctacaccacagccacagcaacacggaatccgagccacgtctgtgacctacaccacagctcacggcaacgccggattcttaaccccctgagtgaggccagggatcaaacctgtgtcctcatggataccagttgggttcattactgctgagccactacgggaactcctctttagtttgtttctaaatctaagtattggctattttctctatttcaaaaaatgaatagtaggagttcctgctgtggtgcaaagccatccgcagtgtctctggaatgctggctgcaggtttgattccccagccggcacagtgggtgaaggacccagtgttgccactgctgtggcctaggtcacagctgtggctgggattccatccctggcccaggaactccataggctgtagggtgaccaaaaaagagaaaaaagaagaagagtaaagaaacaaaccaaagaaaaacaattgccagttaatcggaaatgctggttagtatttcttccaggtgagttttgtattggacgtaattaaggagactctaaaggagcctacagctcgatgctgttccagtatctcgcccgagggtgatggggcatttagtctttagggtaagttgactttgtgtcattcgtctctttctttccttgtttagggatcgcggccaggtgtcgggccttactatgaaaaccacggataccaacctgaaagtgtctaccccccgcagccccccggggcccacaggccctatggggcgtatccggctcagtaccacccgccctcggtcccccagtatgccccgagggtgcagacgcacgcttccacacccgccgtcgtcgtcagcaggcagcccaaaccccgatccaggacgatgtgctcttcaagtaggacctttttgtgtttctctctctaacccgagttccgggtctgccttcgctcttgagctctggtgccggctcgcctctgtctgctggggatgtcctgtccctgcctgcctctccctccgcagctctgtgtccaagctgggggccctggggctcgcctgcagcccagagcctttggggaccactcggctgccaagaccctcatcttccattcatgcttctggaccagcttggcctggaccagctccccgcaccccccacccccaccccatggtgatctttccagccaggtgtctctgacgtgcctcctgcctgtggtttggggcccgaccatcttgaacagatggtaaggcacccttgcaggaataaacgcgctaacccttctctgagcaccactcactggacaccgcacatcctgctcaagtgctggatgagagttttgtgttcagtgagggaagtgggtggggtgtcgcccacttggcttgcagatcttgcagacagaccgcagccggctacggggcatcgccccatgttctgtgcgcatcgccttaacctctcatccatctgtttcctttgcatttcattttgtctcctttcctgccagaacccttcctcttgcaccccctcccaacttttttttttttttttttgctttttagggccatacctgcggcatatggaggttctcaggctagaggtcgaatcagagctgcagctgccagcctacaccacagccacagcaatgctccagatccaagctgcatctgcaacctacaccacagcccatggcaacgctggatccttaacccactgagcgaggccagggactgaacccgcatcctcatggatgctattcggattcatttccactgagccacgacgggaactgccccctcccaacttttagcgcaggctagttgacctggtaggagctgcccaggcaggactagtgcagcagtggcctaggctagctagtattagtgggagcccagagccagtatggtcgctggaccaaggagcagcctcccttatgatgtttgtttgcttttgtctttttctcaatgacgtgattacatttgtagtttacaatgatcctcacaatccagttttataggatttccatcccatacccccagcgataatgtttttagtttatgttgtgagagtgaacctccacgtgtcaccaccaagccgtggtggtgaggacaagcgagttagacccggccacctcagctcctcacgtgcaccatccccaggtccccgcctgtgccttcctctggctccacaagcggctctttctctccccgcaccccccgaccccactgctgctctccccactcctgcgttttctacccgtcgccgtctcctcctgaatcacccctgctccttccatggttggtccctcttttcctccctcagtttccctcggatcaaagaaacacgcatctctgagccgatgtctttccagtggtggctggggaggctgagcccagaggctcccatcaggctgctcctgatgccccagggtcgtttggcaatgtctggatccagtttggggtgtgagaactggggtgggggtcctgctggcatctcctgggtaggtgccagggctgctgctaaacctcctccagggcgtagggtgactgccctccacaaacgacctgccaagatggcagtggtgcccaagcttaaagaccccagtctttgcccatcttgtgcttagagtgaagccaggttgaggaggcttaaggaagtgcggacagtggtacctgcctcccagagaaagaagacgggggtcagaaaacccaagggtcctccagaggtcaccaccttcaaaagggagtcctcgcatgggtgactcgtgcaggtggacaaaaggaagccccttgcctgacatgcgtgtttccgcttttctgggggaaaatgtggtgacgacaggcacgtggacaggagtatctaaatctcccatctgtttggttttttttttttaattattaatattttttgtctttttgctttttctagggccgctcctggggcatatggaagtttccaggctaggggtctaatcggagctgttgctgccagcctatgccagagccacagcaacgcaggatgcgagccacgtttgcaacctacgccacagctcacggccacaccggatcgttaacccactgagcaagggcagggaccgaacccgcaacctcatggttcctagtcggattcgttaaccactgcgccacgacgggaactccccacccgttactctgtaatggagaacgtgcacatatgattgtgcctgataaaatcaccctagatattcaaaaatagcttagtgaccttatttgggtattattttttaaaacatagcatatagaaacatatatgtgacttgacgtacagagaaacatgtacatttataagtgtgatgcttgattttggaagtatttctttttaactaacggacgattctttagagtcattttaggttcgagaacagttgaggaaggggttcccaggcaccctctcctgtcatgcccaaggcttcccgccctccagctggccacagtgttaggatccacgacctcccattgaaacattcttattgttataagttttatgttagggttaatttttttttttttttttttgccacacctgcaacatatggaatttctcaagccagggactgagtctgagccatggctgcgacctacaccacagccatgggaacgccagatcctttaacccaccgtgctggaccagggatcgaacgcacacctccacagagacccaagcgcctgtggttggattcttaacctactgtgtcgcaacaggaactcaaaggattacattcttttttttgtctttttacagccacacttgaggcatatggaggatcccaggctaggggtccaattggagctatagctgccggtctacgccacagccacagcaatgcaggatccgagccatatctgcaacctaccccacagctcacggcaacgctagatccttaacccactgagtgaggctagggattgaacccacgtcctcatggatgctagtcaggctcatgaaccactgagccacgatgggaactccaggatcacattcctttttgttggatattcaatagattttgcctaaggaataacaaggtggctgtaactcaagtgtcatacagagtaatttcactgccctggagaccctttatgctcagcctgcctatccatctcattctcccccaaaacttgacagcttctggtcctactgtctccgtagttttgtcttttccagaacgaagagttgaaatcacgcggcgtgtagccttggcagtagtttgcaagttacttaaataggacacattcaagagaatctcatgagaagcacaagggacatttttggacacattggatcgatttgtttcagcaagagtcccagatgtttcaccttgaaccacaaggcagacacgtcttagtgtatgggttttgggtgattgaagttcaacaattctgtgacgcaaattgttgacacaggacatggcagagctgccacaacaataggaacaatggataacaacaatgacggtaaatgatcaataagttataactaaactcacaacaggtgcaattgccaaccagcacccaggcaagtctgacctaaccgggcacttcctgtccaccaaaggtgagaactggggctccccctgcccacgctcctggtgagagccaggcagactggcatccgttcaatgggaggctcaggataccccgtgtaaccgccctcactcatcaggtacattgtttgctcttggataagatgaaatacaggcaagtcgcccagaggaaagagccatccttggctaaggtgtccaattggccattagctcgctaggtgaccccaccctgcctgctgtcagatgtaaccactgtgggcccagtcctgagaacgtggtcggtggcaaatggtgctcctcggatggtggccatgaccttgcctgtgagcctgtgggactgcagactcaggcctcccgtccccgcgctggggcaggatccctgattaatgggctctctagctgattcccaagcctgcctaagaacagactgtgggcgttcccgccctggcgcagtgggttagaaatccgactgcatcggctcagctcactgtggaggtgtagattccatccccagcccggctcagtgggttacactccagcgttgccacagctgtggctcagattcatcctggaaacttccatatgacgcacctgccatacaaagaagaaggaaaaggagttcccgtcgtggctcagcggttaacaaatccgactgggaaccatgagcgggtccgatccctggcctcgctcagtgggttaaggatccggcgttgccgtgagctgtggcgtgggtcacagacacagctcagatcccacgtggctgtggctgtggtgtaaactggcagctacagctccgattcgacccctagcctgggaacctccatacgccacgggagcggcccaagaaatggcaaaaagaccaaaaaaaaaaaaaaaagaagaagaagaaagaagacagtggaatttgcagcctgtggtgacaaaatgagtggcgagtcttcgagatccaaatcaagtgggtcagctctggtctctggccccagaagctgaactccttggcctggttgactcctggaagaaagacagtgctcaggtcggggccaacgcccctccacacaccccacctctgcagcctgggtgtctcccgtgtgaaccggggagtccaaggctccttcaagccctctcgcccctccgcctctcccctgcttcccaggggaagcgccaggagaccctggcctggggccgcttcctgcgcagggtctgctggggcagctgtgtctcctctgtggcatgactttgtctgacactctgaccaacaacggctcgttagttttgactagagagccctggaggtactcctagctgcaatgggagcctgagtttctgcccccggacccaggggcagcggcggcgctgcccaggagaactccccgcagtgatggaacgggcgcctctgtactgtccagcgcaggagccactggcgacttttgagcatctgaattgacagaatgatttaatgccacttaaggataattaatgggcgcgtgtcgacttaagaaaataggcacaacttaaaagttgagagttaagtttcactgggggcaaaatgaggaccagagcctgggaggcggcattgctgagagctctgagctaccgccccagagaggcaggggaggtcagtgtagacgtggttttggtgaagggggaggtccatgcagccaagcacacactttaccgaaggttattgctcctcccctgaaggccactgctggtcacgaggagcagatgccaccctgacggattttagtgcttttctagatacgaggagatgcaagaactgggctcataaaatctgaaaatatctgaagacctgttctgccagttttctcagagcacagaaggccctgctcctggtccccatcctgagttttcatggggctgaaggttggcagctgcagcggctcatacttcaatccctgtagaggcagccggcagggccgggtccagttcacaggtgcaaagagctatgtgtggctttcatgagtccgcgtggctagcgtgattctgtcccatccattccctaaactgccttgggaaagaagatttttttcctgctgccctgagagcactaatgacatggaggtgatagttttgggggcattactgttattctgcttaggctcgtaattcccagaacaagctacttttcctttttcagccatttttttccattgctcctttatttgaatattttaaggctgtacttttctaagagcttaacttttgcgtatgttgcttcagatctcggcttcccgctttggttctccaaaaagcatcaaagccagtgccaaggaagaagtacgacctcctaggatgtgtttctaaagagaaaagtcacatttttaagtgtataggaaattatgattccctgataggatagaatataaaaatatggatgaaattcgagagtgtaatttctcagaaactcggccactaaattgtttactcaggagttcccgttgtggttcagtgggttaaggacccagagttgcctctgtgaggacacaggctcaatccctgaccttgctcagtgggttaaggatccagcgttgccacagctgtggatcgcagatgcagctccgagtcgccccctggcctgggaacttccatatgctaaaaagccctaaaaagaaaaagtttaaaagtttaaaataaataaatagtttactcacctgttttcccccagagaccaagaaagcgctgtgcatcacctttgctctgggggccatcctggcaggagcagtgctggccactgtcctgctctggaagttcagtaagtgcgggagagggtggggggcagcgtgctgggctggggggcccccctgcagcccctccggaagtctcccatcgccctccttccgtttggttccgtgaataaccttcctcccttccagcctcgtcctcgcctgtaggtggtcgatactacctgtgtcctttgagtgctattgacacacggtgggagcagtgggctgagtccaccctgtccgtgctgggggcagtaggtggcacctggcactctgagcttacctggtccgtgtatctcagtgcacagtggtggctgctgcaggctggggcgtggcctccccaggcacctcctgcagccacagccaggtcacctcccatagcccagggcaggggggctgatcccaggacccccctgggtggccgggaggggggcagtgctgtccctcccccggaccggaagtcaggggctcaaggggttcagccccccttccttccacatctgctgttggcaaggacacattcccttctgttaaaataaagggggagggagttcccgttgtggcgcagcaggttaaggacccgatgctgtctctgtgaggatgcaggttcgattcctgaggcctcactcagtgggttaaggatccggtattgctgtgagctgaggtgtaggtcgcagatgcggctcagatcccacgttgctgtggctgtggggtaggccagaagctgcagctccgattcaacccctaacctgggaacttccatatgccgtggctgtggccctgaaaaagaaaaattaaaaaccaaaggggggaatcttattccctcttgttgcgtggggcacagagggtgcctgacggagctcccgggtggcgggcgccgaggggctgtcttggtttcccagcctgacgggcgtctccctcccgtccctctgcaagtggagaagaagaggtgctccacgcccgagatggagtgcggctcttctgggacctgcatcagcccctctcactggtgcgatggcatcctgcactgtcccggcggcgaggacgagaaccagtgtggtgagtgacagggccgagcccacacgtcaatccctctgtcactgaacccctacctggaggccgagcctcacctctaagccagtctccagatgcggagacagggtcagaggggctgagccaccgcccagcatcacacagcctggcggggagggagaccacagcccagggccaggccaacccacgtggccccagcccccccaggacatcactcctcggcagctaaaaagagctgcgcatttaaagttgggtgagtgctatgtgtttggtgaacaagctgcctttcaaggaatctaggctattgtcacagggcgggtaccactcttgcttttattattttctattaattatttcaagggtggtaacgtggccgtgccagggaaagcgatgtgtgaatgccaggattgtgccgatagcccctgatggccgtacccagagctgatgcccgctgcatcctgagggtgtggactttgcgggcgaggctagacaagggcagtgttccagcggccaccctgggcgagggttctcctgcgtgtccccactttcaggtcagggcacctgtgtgaggtgtggggccctggctgttgaatctctcatctgggagactggacagagttcagcctccactgcccgagtggagcctcgtccaggtcagcgcctcagcctctgtgcctggagcacctgctgatgccgcctgtgcccggagcacctgctgatgccgcctggagagcgtttgcccttggacgtggctcactcaccccgtaaactgtcagcatagtgccttgtctttattttggtgatgatagatgtgtcagcatttatttaagaaaagcagggagtttttgaggttgagcagccgtggttcacataccggaaggaatgacctctgtcctacacgtgcacacgtgtgtgtcaataaatcctagcaagacgaactcatttcttacctgcaagactccctctctctcctcgtaagattgaagctaaggtgctcaggtagatcacaattcaggcggcacctgagggcgtgcagtgaaaaagctcatcccacttcacttacgcccacgcccactcacacacccacgcacagacacactcacacatatacacacaccctcacgcactgcgaaataatagaaggtatgcattggtctctgcccccaggtcccagcaccgagctcccgaaaccctggttactcctaagtggccagagcagcaggagcatccttagttctagtacttggtccttgacctcagttcctgacacaggactcctgaaattctcatcacttcctgggtgatgggagtgtcttttgttctaacgaggtgatttgggtggaatcccagctgggggctgctcaccacaaagaataagccgtggttagaagcttggacctgtcagtcctacacctccgtctcttgagaggtgagaggggctgaaaatggagttaatgtttgatcatgccttcatgatgaagcctccgtaaaatcccaaaagcccagggttgggagcttcctggtgggtgatcccaggaagatctggggagactggcacgcccagagggcacagaagcgctgcccttcccacatccctcgtcctgggcatctcttcctctggatgtccacctgcatccttcgtagttaactggtaaacggtaggacacggttttcctgagttctgtgagctgctctagcaagtgaatcgccctgagcagggggtcacgggcacctctgatttaacactggtcagtcagaagcgcaggtgacaacctggacctgcgactggcctctgaaggggggacagtcttgggggactgagcccctcactctgagatctgacagctctccaggtagacagggtcagagttcagctgtaggacaccacctggggtcgtggagaattgctgggtgtggaaaaaaaccacctcccccccacaaggggtgacaggctgtgtgtgtcaggagtgttcagtgcgagccgtaaaagacaacagggaacaggacaccaggaagaggaaagatagggtgttttccaacacacacacacacacacacacacacacacacacacacacacacacgcctgtccaccagccagtcatttcttttccttacggggcaactaatacatgttatttctctttgcagagagtttttacacaaaaacaataaatacgtgcttgcatttttttttttttagggtggcacctgcggcacgtggacattcccagcccagggattgattcggagctgcagctgccagccacagccacagccacacaggatccaagccgagtctgtgagccacaccacagctcacagcagccctggatccttagcccacagagtggggccagggatcgaacctgcattctcatggataccagtcaggttcactatcacagagccacagtgggaactccctgcccccgcgttctgaatctgcccacttcccagtggtgactgcccagccgactctgcaggactttacatcctgaaagtcgtttcctgtcgggacaggacccaccccttttcctccgctgccctcgcggtgttcctgtgcttggaaaagggagccgtttcacttgtacagaaaaccggtgggcaggacaggattcccacgcacccccccgccccgagttgccccttttaacttcatgcatcaggtggtgccctgagagccgtgagtcaatattgatgcgttactatggattcaagtccttgagcgcgttaaggtttgctcttgctgttgcagaaaaatgtctaatgtatttacatttggacagcgggttgggacaaggtacagcacgcacccactgccgtggcatcctgcagagcagtttcactgccctcaacaccctccgtgccccttccccccccactccccctcgctggtaaccactgatctttctgctgtcttcgtggtttggcctttccccaaatgccacggcatgggaattgcacaggcggtggccgtttcaaatcagctttttcatcactttgggacgtgcgtttaagggcctccggtctcttcatggtttgggagcaccgttctccgtagagccgaatcatagtctgttgtctggatgtgccacggtttgtttatcctgtgatatggaatcgctcaccgcggaagcgctaggaaaggacccgaccaaggctggtccttcgtggaggcgtgagccccagatgtcaggctgcccagggagtggggtggggggctgcgcgggggagagcgaggcctgggagcagccgggtcctcctgttgtacctaagcccccgtgtctccccccggccacggagccccgtctccacgctttcaggctctgcctacgtaaatggcacagggagcacgtcatttggtgccctgccgtcctttctgcagtgccgttttccagccccttcatgttaacacagctccctggttaatccctccacgcgcaggccttcgctctgggaataacacgtggagtgtttctcctacaccaggaagagagtcagaattctggatcctgttacctctgccggcagagaggggctgggacagggagggagcttcagctatgtttataatgtcttacctctttaaacaaaagggcattttgttttggcaaatgaaggtggaggtggagctttacataaaactcatcgctgggcatggccagggcacaggtttgggcagaggggtccacctgctgttctgtgtttgtctaagaagcagttttcaaaaataaacaaaccaaaaaaccccaaacaaaagaaaaaagcatttttcaaaaatgttttatctgctttttttttttttttttttttgtctttttaggactgcacctgcggcatatggaagttcccaggctaggggtcaaatcagagatgtagccgctggcctacgccacagccacagcaatgccagatccgagccgtgtctgtgatgccagaccctcaacccgctgagcaaggccagggatcgaacctgtgttctcatggatactgctcagatttgtttccttggcgccacgacgggaacacctatctgatgtttgaagaaatggctctagaatatgtatataattcacataccacgaaagccgtcctttgaaagggcagatccagagccagtggtcttttacctgcccccacagacagcgtcactgtcaccgcagtcacttggggcccatctccttcatctcctgaggagccagctctcggcccagcccctcctccctctcccaggccctaagcaaccactggtctacttttggtttgtagatttcctcttccagacgtttcctagcaatggggtcattcagagtgtgggcctttgtggctggcttctttcactcagcaggatgccacgcgtgtccacggtgctgtagcgtggacgagtaatggtgcgggtgggccgtgttttgtgccttcacctgttggaggaggtctgggggttcccaccttctggctcttgtgccgcgtgctcccacgaacatttgtgtgtaatgctcgattctgtctgaatacttattttcggtccttttgggtgaagggcactttttgtgggaaagcctagattcttggcatccgtcgtgtgtgtatgtgagtgtgggcgggcggagggagcctggccgtgtccaggagggtggggctcacggggcagacaggactgacagtggcctggaactcaccagaatctccctccctgcagttcgcctgtacggacccaacttcattcttcaggtgtactcggctcagaggaagtcctggtacccagtgtgccaggacgactggactgagaactacgggagggcggcgtgccaggacatgggctaccggtgagtcggggcccgcggccccgtgacaatggtggcagatagaaatgtaagctcagggcttctttcctggtccctggaagggccggcacagggacacgagtccctcccccaaagctgctgcaggaaaccagcaaggaaacaggggcacccccaaccccctccctggtaccctcctctccagccctcctcctggccctccctctgctcctggttgcccagggctgtccagggctgtggtcaccagggtccggggtgccggcccaggtcctgaagagcctccagggcaaggtggaggccccgactccgcccacgtccttgctgggggctgggggcctgtggtgggggctgggggggtcagagaggaggggtctgcctgcacttgacggcaagctcgcaggaggccctgaggtttccaacagcagagatcatcagatcccgccggaggagaattcacgcctcctgcagccaccagagcaggtgcgcggggggcgccctcgccccgggatcccagccgggggctcccaggggaggacctgtgatttcagtgccaccgtctcttccttggggcgtcccgtgtgacctctgcgcagtcctagcgtgttctctgtgtgccagtcttcaaagagaactgccgcttgataagaaatccgtcaaaatgccgtgtccccagttgtggtatatttaacaataagtgacagtgaggcccctgtcacatttcaagatctgtattttataactgggagcactttaatatctgtaattcggttaaattacatctcacacaaccttttactcttactcctttgtcctaatccagtttattttatttttttatttttatttttttgtcatttctcgggccgctcctgcagcatatggaggctcccaggctaggggtcgaatcgaagctgtagctgccagactatgccagagccacagcaacgcgggatccgatccgcgtctgcgacctataccactgctcacggcaacaacactggatccttaacccactgagcgaggccagggatcgaacctgcaacctcatggttcctagtcggattcgttaaccactgcaccacaacgggaactccctcatccagtttagattagaaacctggctggtggtggagttcccactgtggctcaatgggttaagaacctgacttcattcctgtgaggatgtgggttcaatcccttgccccgctcagtgggttagggatccggtgttgccgcaagctgtagcttaggtggcagatgtggctcggatctggcgttgctggggctggggcggaggctggcagctgcaactccaattcaacccctagcctgggaacttccacgtgccttgggtgtggccctcaaaagaaaaacacacacacacacacacacacacacacacacacacacacacacacggctgatggaggacttttccctgagacctcaaggttgctggatacaaagttggttatcacacagtgtgtgatatcacttgattcgctcttacgtttttgttttccaagcactaaagtattcctctcaacccaaacgtcaaacagagaatatcacgatgcagctgtagcgtgggaactatgactgctcacaacaaaaccattattttaaatgagattttctgtctctttttaggaatagctttttttctagccaaggaatcgccgatgacagcggggccacgagctttatgaagctgaacaaaagtgccaacaatatggatctctataaaaagctgtaccacaggtacttaattgggggatttcttttctttttggccacggggcatgcaaactcgcaccagggatcgaacccacaccactgcagcaaccccggccatacagtgacaatgctggatccttaatccactgcaccgccagggaactcctcatttggggagttcttttttccttttcagaagttttatgctgagagtgggtaggaatccgtgtccagggtttgtcctctctgctgcagttgtgggtttggctgagtttctcacagggagtggattggacccaagtgggacccgttgctctgagcagctctggggtcattcttgggaagtggctaaggcgtattggctgtttagattgtgccctctgccctccatgctattgggtgacatcgaaaaattaagattcttctttcctgattatcttccctctccagatatcagagatgaactttactgagttgtcggggggtgtgtctcggaatcaaaaaatatgtctcagggccccaaatggagtgacctacccatgctgctttgcccaggactctgcgggtttcatgctgggaatcctgcttcccaggagtccccttagccccaggcagcaggggcagttggtcccccagcccccccagcccccagccctatgtcaggatgctgtgttgggggaacctggagccttgcctgtacctggggacagcttgtagtgctgggcgtgtcatcacgtgactgatgggtggaaagggagccaggctggccccatggttgatggtctgtctggctaggagtgtgggggaaagggctgggcttaagccactcattttgtgttgccttcgtttaaaatgtttgcggtcttcctggcaccatgactatcctccgaatgttctttaatataaaagatacacagcaccctttgtgttgaagcctcccttgcttccaggtggccaaccaggtcgtcacctcccagcaggccaggtgccaggtgctgcatctggggggacaggtcatattccccgccttccctgtgtgtgggaggctggaagcaggtttgcattcctggtggcccaggctgaggtgtcgaggggctgcctagtgccctgccatccccaggaacgtgccagggtgagcccagaaggaagggtgtgcccggccaggtgcacccatcaaaacagggggctgtggtttgcccgccccagaagctaaacacagctgtctgggctctttgtaaatgctgagtgtctccttgtctctctgggttacatggcatcagggcatggggttggggggaggctgccacccctcagagccgtgggaacccacacaaggcgctgcaggttcagaggaagggagagaaagtccccctgtctcaaggcgtcccctatgtgtgccagggggtaggagtgcttctttcctctttgttttcagaagttgctctttggggagttcccttcatggctcatcagttagtgaacccaacttggatccatgaggttaggggtttgatccctggcctcgctcagtggttaaagatccagtgttgccgtgagctgtcgtgtaggtcgcagacgcagctcagatcctccgttgctgtggctgtggtgtaggctggtggctgtagctctgattcaacccctagctgggaacctccatatgccgtgggtatggccctaaaaagcaagaaaaaaagaaaaagaaagaaaagaaaaagagaagaagttgctctttgagggagttcccgttgtgaccacaggtaacacatctgactagtatccatgaggacgcttgttcgattcctggcatccctggcctcaatcagtgggttaaggatccggcgttgccatgagctgtggtgtgggtcacagacaaggctcagatctggcgttgctgtggctgtgatatagactagcagctgcagctctgattcgaccccaagcctgggaccctccatacgcgacaggtgcggccctaataaacaaacaaaaaaagttgctctttgattactgagcctcggactcccgcggggtttgtcagtggcgaggggctccctcgtgcctccgtgctggtggccacacagcatccatccatgcagacgagaatgaccttatcatccttagacgctgcctcatggaaacgtgtcttctcttggcatctggcgcccctcaaccagaggccaccaggatcccttgtcctggaaccacggccgtcaccatgggacggtgttagaagggacttattacgggacttgtgggcttctgttgcatcgctttggggaaggttccaggaatcagggctttgccctagattggacccatctggaagcgaggatgattctgtagttggcacgttgacttttgtttgttttttagaaggtgggtcacttgaagccaggctatatacagctgggattggcaaagaattgtgtcagccagcagagggggctgttgggcactgcctggtttggaccaggtccgccgtctgtgttcagaggggctggcaccgtgagcttgcctgtgttctgcttcatcacgtagccatgatgttgctgctccgtgagctgtttctgttggacagaacacacggccagccagccgcaagcttctgggtgtcagggctgcttctcttctcagcgtattaggggaaaattcactctgcaacagaatgcattggttatttctctttcatctgtaacctggaatgtctacctccccaaagactcacttctcttctgctcccaggcagaggaataattgtatagtttcctgtgcaatatatgtagagcagtgatgggaagcaagcagtttacagatatcttacttacaaaattaaagggtgattttaaattccaggaaaaaacatcagagccaagatgatatgtaccgaccttcacaatcgctctggtactattcttagcccattctacagatggggagtttgaggcacagagaggtgaagtagcttgcctaaggtcacaccgcttgatttgaaccctgcactttgactgagtgtctgctctttactgctgtcccccacgtatatagatttactttccttttatttttctttctttatttattgctttttaagggccacacccatggcatatagaggttcccaggctaggggtctaatcggagctacagctgccggccacagccgcagccacagccatgccagatccaagccgagtctacacccaacaccacagctcacggcaacactggatccttaacccactgagccaggccaaggatggtacccacaccctcgtggagattatgtcaggttcttaacctgctgagccaccccggaactcccaactgcagacccttctgtgtctggggaggcagggagtgagaccagaaaatatgcattcccaggtatctttgagaatccatttattgtagtgactagaatttctcctcttcttccgtagtgacgtctgtacttcgaatacggtggtttctttacgctgtataggtaagtgactcttggttttccttctaagtaggaaacatggggtatttataggaacacatccctgtctgtctctgggggccatggagagagaaggaggggtacaggtgggcccgtgcccagcgctctgcccaagtggctcctttgctgagccctgatgcactcgagacgcctatagcccaggttctagaagcagctgtcccacgaggatgggcaagctcaggaaattgacagggctgctccgtggcgtccgaggtggagccggaggcccgtgggggtctccctgcctgtatttggatgacttagagttttgaatcttgtatcagtatgggggacactgggatcctttctctgggacagtcccgtcctgctcccgtacagatctagggcctgaagttcggcttttcagttcgagtgggtgaagctagcaggcaacccaagtagactcatgcccagtggtcccaacagtaacttcctacccaggctggttcaggcttccttgggtaccaagaaggcatggtgcccaccccgcctcctgcccacaccatccgcctcctgctagcctgagagcagcatagacaaggaaagtgtccctggctttgttcaggaatcctggggcctctgagcgttcaaggagaacccggcctttgccatccagcccccacgcccccacccctgggaattcaggagatgctgatggaatcacacaagtgccaagtagcaggtggcattcagtgcccgcagaagtggtgcttggaggggaggggagggtggtactggaggagtgtcctccccctgtccccaacccaaagagaccaggacacatttcctcaaagtcagacctgaaacaaacaggaacgagaggtggcagtagtccccctctggccaggcaggactctgggggatgggagccatctgtgtttggtggcaccacggtggccgggcagccctgcttccgttcactggctgcgctgggcttgtcgcctttagagtgcggagtctccgggaagatgagcaaccgccagagccggatcgtgggcgggtccagcgcagcgctgggcgactggccctggcaggtcagcctgcacgtccagggcatccacatctgcggaggctccatcatcacccccgactggattgtgacggccgcccactgcgtggaggagtaagtcccgaggggcggggcccgctggcgggggcggggcccgcggggggcgggggcggggcctgggctcatggtggctctctgccaagtgaccttgagcgaagcctttgcgttgttcacgctccagcttccttgtggacaagtggggactctaaggcgcttgcgtcgtggcgttgctgggagggtcagttgacttatgaagccagtggccaagcccgtcccttcagcgcagggatgcggggttgggctgcgtgatcctgcgatcagaactccttctggcccgaggcttactcagctccatcctacccttcccggcccctgccctctccggaggcctggtctcccctgggtgcactcagagacgctcaaacctgatgctgccctgctggaaggttctctgtgctccatgaaacagcctgggccacagggttgtctgtacagatggcgggggagacgcaagggtagccatcatttacagcgttagcgtttgtaaaaaatactgccccccctccttttttcttaaacatgaagtttttgggtcccatgcttattttgcagaaaacctcactcatcaaagctatttttcttgtttagagtagggtgggcctggcgtggctcctggcccagctgttctcgggctgagtggcctcagacagttcactctaggtggctgacgttaccacctgcacctggaggacaggtgtgccgctcctcccaaggctgctgtgatggatgaggaagaggtgtttattagccaagagtgtcccctgccttccatccaccagattccaagggctctggtattttggattctcttagacaggagacatctagaaaaggttatgcccgggtggcttttagactcagagtctgttcatcactatgcatttattaagcacctactgtgtgccaggaaatgctctagcatataaagctaagctccaggcctgccagaaatggggtttcaaacctcagggaggcctttaggctcaagagtaagtgttctaggaattgcgagggtttgtggtctggggccaacacacagggggagcaggatccattgatgaggttcgggtggggggtgggcagaggaagctgcgagaggtcgggggagagaagactccaaggccacctggcggggcttttggcaagtcgtgtcacctcggggacccgtggctctggctgatgactttcaaagacccgtgacggagggcttctggtctgtggttccgagatgctgagtggtgctggtttctgtcttccagacctctgaacaatcccaagatctggacgcctttgcaggaattttgagacaatcgttcatgttttacggcagcggataccgagtggccaaagtgatttcccacccgaactatgaccccaagaccaaaaacaacgacattgctctgatgaagctgcagacgccaatgaccttcaacggtatgcacctgtgtccctgaggcccagggtgtgcgagccgcttggaccacatcttaaggagaggttgatgggaacgaaaacgtgctgggacgcctggaaagagggaacatgcactgagttacagcgtgacgagctgggggagggggtggctgatcaaagaggactcagcggaggctgagtcactcccgaaaccctggggccactgagggacacggtcaccaccgtggttttccttcaccccccagagtgacaacttagcacacataacctgctaggaaagtcattgcagaatcagcgtctggtcggtgagcctgactgggggctggctgcacctgttgcgagtttaattctaacatgctgctcatttaccttcatgcttatcaattgattctgctgctgatgctgccctgccgtgtcaactattcgcatctctggaccgttggcctttaagcctccaaaccccaaacttccagagggagtttgctagaaggaggggcctggcaggtttcaggaagagggcgctaggataggttggaatctgggcacctcctggggcaggaggatggtggtcccaggcccagcgcttgcccaggggaagggcccgagggagacggtccctgcggcctcgggtggaccagacccgctcaccatgctttcccttcttgaaacagacaaagtgaagccggtgtgtctgcccaacccaggcatgatgctggagccgacacagtcctgctggatttccgggtggggggccacctacgagaaaggtgaggctcccggcacccagatgtgcaggtcccacgaagccgagcaggggctcgggagtgagaggtctaggttgtcatcctagttctacagctaaatggtcggatacctttgggcaattcgcctaatctgtttgcctcagtttctccatgtgctagatgggaaaatcccacctgccctacctaacccggtgctcttactaagatcaaatggaacaatgtgtgacactgcttcttttggggggggcacttttttgggggggcttcacccgcagcatatggaggttcccaggctaggggtctaatcggagctgtagccacgtctacaacctacaccacagctcacagtaatgccagatccttaacccactgagcgaggccagggatcaaacacacaacctcatggttcctagttgggtttgttaaccactgcaccatgacgggaactccagaaactgctttttggagtgtgaaggccacagaaacgtgagaggcacacggccccctgtgtttgcacaccgcgtggtaatgggccgaacaggaagtcgggctgcctttgatggctgagcaataatggacttggtgaggatgggcttgagtgaccagcagtggccgacggcaggctgccaatgcctttgtccttctgccttgaccccacagagatggagcactaggctgttctgtcgcaggctggtcccgcttccttcagttctaatttgctttcatctccagtaacgtttttcttttttttttttttgttttcccactgtacagcaagggggtcaggttatccttagatgtatacattgcagttacagttttttcccccaccctttcttctgttgcgacatgagtatctagacatagttctcaatgctattcagcaggatctccttgtaaatctattctaggttgtgtctgataagcccaagctcccgatcccttccagtaacgtttttcaaatgaagggatcctggctgtttcattttcatggagcgtgggcagtgcttagcagtacattttatatatttgttacatccttccaaaagggtctggggcacgtacacaaagaatcctttaaagcaaggagaatggcacaagggtgggctgacttggagggaatggcggtggtgctgggtatcctggcatgggctcctgaggttgagtctttcagttctcggcaaggtttccctgttgccaagatgaaggggaaaatcagccccaatgtgagcatccacttcaggctggaaatgcagcagtggacaaaagaggacaaaaatctgcccgcaggacgtctgggggcaggggtgtgttcagacaggaaacagaacactgaaccaggtgctttcaaacagtggaggggtggtcagggaaggctcctcggaagaggtgacacttgagctgagatctgcatgaaagagggtcttaggaacagggtcttaggaacttaggcccagggcagctggggcatgatgagctggggcaaggggacaggctgggaccaggttgagtaggacctcgtacataggctatgatgaggacgctggcttctaatccgagtgagacaggagccatcaggaagttttaagcagggcagtgacaagatcccacagctgcgtagggactcaggaaaagggtgggggcgagggcagacagtgtctgcctgtgcagcggggcctgtgccgtgtcctccgaggccggaaggtggctcagaccagagaggtgggtgcagaggcccatttgggacagagccacctgatctgctgatgggtggagtgtccccagggagagggtgaggggtcatgggaggaaggcttctaactgatgatctgagccccacctccagtggtgatgccctttcctgagtccggggggactgggtggtgtggctttggggaggagcacgtttcaggagagaatctggtttagcatgttaagcccgcggagtctagaagacacccaagtagcaagtggaacctggggttaagacagcaagtcccagcgagtgtctggaaagcttctacgggcagggagccgagggtgtggtggtcaggatggtgtggggtctaagaagggctgtttgtaagaggtgataacagggggatgggggggcgggctccgcgtggtccagggaagagggagtgatgaaggaggaaacttaatcttagggaaccaagtggctgggcgcctagagagagggcgatgctaagtcatagatgtaagtctacagccggaggaagggcagccttcgcccccaagcgcagtctacagcagcgcgcagtacaggctccagcagctggaagacctctcggttacccaggaaacagttacccaggaaacggttacccaggaaacggcgtgggagtgggggcaggagaggggcggggccgactgtgcacgtggccaggaggggcgtggccgccttgcaagggccccggagcggctccaggcttgctgtgagagcccatgccctcctctgccgcagggaaaacctcggaggtgctgaacgccgctatggtgcgcctcatcgagccctggagctgcaacagcaagcaagtgtacaacaacctgatcacgcccgccatgatctgcgcagggtacctgcagggatccgtcgactcctgccaggtagcggccagcttgtgttcggccttggggccttgggagccctggaggagagcccctcccccgcctgggacccatgggacgttagggccatcggaggggcggcagcgccttgtacctcccactccgcccaccgctgcatcccccgaaagggtttgtttggtcaaagcgtcattgtagcagaaataagtgttttgattatttttaaaatgtagacaatcatttatttggtaacttagctgatgcagaccagaactcgttgccagtagagtctgttgaggggatgatattactgtcaccttcttggagatgagaagcaggaagacaccccccccccagccctgtcacccaacctccctgctcttggagggcacatggcttggggtttggtttctgttctctaacttctttgtcccccagggtgacagcggaggtcctctggtcactctgaagagcagcatctggtggctgattggggacaccagctgggggtccggctgtgccaaggcttaccgaccgggagtgtacgcgaacgtgacattatttacagactggatttatcgacaaatgagggtaactctcccgtcctcttcttgggggtctctgattctctgtgtcaaagcccagacacctggaggtatggtcccatggcatgtggcgtcccctcccccaccgctggtatttggatgaaaactctggggctcagctctgcagggtcccggactaactgtaatgggcttccttgtgtgctgcggaccaggcttcgttctggctcagctggctgtgatcacccacccacacggcaacaaaccaaacttctgatgatgggcaaggagcagagggaggcaggtcagccagcggtgactctgccctgtcctgttctgggcaatcggatgccccttgggtgggggagcaactgaggttctcaggtacaaggagaggtggtgacgaggggagaggtccccggggttcctgctatcctgccatccccccgactcttgccctggtccaagatgctcaggtgccctgtccttgggctctgcttgaatcctgggtgaggatcagaggtctgtccatcacaggggccttggaacaggcagcacccacagatttcggctccttctagggctaggttcgcggtggaagaaaaagctctattttgttccctcccaagtcaccaaaggtgcccagatgccaggagatggggccaaaccagagggcattgcccaataagaccaggcctgcaccttccccgagtgaaatcgtaggacctgccatcttcaagtgaccctggggcctggccagtccccaccccacaaagggcccagtgctggaaataaacaaccccactgtctcgagggggctgagtggtggaaggaatctgggagggggcaggcagggctggggggtgcaggcagccgggaacctagagggagattagccaggccgtggggtgaccgtccctccaggatgagagcgcaaaccacacagtgtgtgcaggtgcttctggtggctcagcacggccaggctgggtggcggcagagaaatcggggtagaaagtgggatcattaaacactgatgtcctggacttcccgtcgtggctcagtggttaacgaatccgactaggaaccatgaggtggtgggttcgatccctggccttgctcagcaggttaaggatccagcgtggctgtgagctgtggggtaggtcacagatgctgcttggatcccatgttgctgtggctgtggcgtaggccggtggctacagctctgatttgacccttagcccctagcctgggaacctccatatgccatgggagtggccctaaaaagaaaaaaaaaaaaaaaaaaaccagcaaaaaacaaaaacgagtgatgtcctgcagtggctgaggtcacgcgtggttgcccagagaggcctgtgtccagctgtgctgcatggttgcatctccctcagggcctttgcatgtgctgttctccccccactgccagcctcaccccttgactttcgtgccgcagaaacaaatacccctctgtcctatgacgcttgctcgttgggtgtacaggggcctcatgtcaatctagaggcaaataccttttcccctttgctggttttaagaaagtaacatcgtctctctctctcttttgaacaggcaaacagctaatccctcgagccttggcccttcgcaccactccgcgagaaaacgaaggcactgatttgtcatccccgtgcgtaacctattttcaggaatgatccccaggtcctctggtgtttatgaaatagtgaacccgtctgatttggcgctctccagggtcctgtgctggctgcgcgttccttcccggcccctcatctgaatagtgaggctggccggcggtggccacccaaggccagtgcagagcagggggtggggtctggcccactggggtcaccctttggaaccccttcccggggggccactcttgcacgccctgggggctggtgacttctcagctgctggacggctggagatggaagagagaaagccaggcagacactgcgggggccaccctcgcgccccccgggacctctcttcccccgagagagtttaatcattctttttaagtgccttcttcgcagccccggctggtgcctggaaataaagggaccagctcttgccggcttatgactccatagccacttgcaaggggcacggaaacctggtgagggtgtagacggcgccctcagagcaggtgcccccccccgcccccatcctgagcactgctgggactgggggccctgtgtgttgggtggggctccacccccggagaccagccttacaccccgcctcctcccagtccctctccccagcctcccgggagactgtgggacaccccccccaacactgtggtcctggggggctggtgctgctgacccttttaagacctgttaatcacgtggaaattgaggtcccccggggaaatcagggatgctgattaaacgacactgtttcctactgtaaaatttctacacgttgctacctcagtgctcacggaatctgggccgtgccccctaaggcgttcctccccttggttctttgaaaccactgtggtcctttgccacgtgtgagcagtggccggcgtgagctgcttttacaaagcgactggctcctcattcaatgttctctacgggacgcactgaagcacggcactgtggcaaagaagatgggggcctcggggctccctgtggccccttctggcccctgaggttccccagagggaaggttgggcctgaattgcggagtgtgacgtgtcaacgcagagatcctcgcagacacactgagaacgaagcgagcagttctgcgctgggacctaagcttggatggaggcagtacgatcccctggcaggatgcatccgtgccacgcacgtcttgggccacagcatccccagggaggggccgtgcagacagacgtgtctgctcccagagccagattttaaaaaaagcgttgtcgcggggtgaaggcatcccttctttagggctccttttaatttatgtgacgtctgttctggtctctgttttgtatctttttttaactgtaaaattcagttgtgaaaaattgtgcaaataaattatacatttttttttt

SEQ ID NO: 438 - protein, 496 aa, Sus scrofa
MALNSGSRPGVGPYYENHGYQPESVYPPQPPGAHRPYGAYPAQYHPPSVPQYAPRVQTHASTPAVVVSRQPKPRSRTMCSSKTKKALCITFALGAILAGAVLATVLLWKFMEKKRCSTPEMECGSSGTCISPSHWCDGILHCPGGEDENQCVRLYGPNFILQVYSAQRKSWYPVCQDDWTENYGRAACQDMGYRNSFFSSQGIADDSGATSFMKLNKSANNMDLYKKLYHSDVCTSNTVVSLRCIECGVSGKMSNRQSRIVGGSSAALGDWPWQVSLHVQGIHICGGSIITPDWIVTAAHCVEEPLNNPKIWTAFAGILRQSFMFYGSGYRVAKVISHPNYDPKTKNNDIALMKLQTPMTFNDKVKPVCLPNPGMMLEPTQSCWISGWGATYEKGKTSEVLNAAMVRLIEPWSCNSKQVYNNLITPAMICAGYLQGSVDSCQGDSGGPLVTLKSSIWWLIGDTSWGSGCAKAYRPGVYANVTLFTDWIYRQMRANS

SEQ ID NO: 439 - protein, 495 aa, Sus scrofa
MALNSGSRPGVGPYYENHGYQPESVYPPQPPGAHRPYGAYPAQYHPPSVPQYAPRVQTHASTPAVVVSRQPKPRSRTMCSSKTKKALCITFALGAILAGAVLATVLLWKFKKKRCSTPEMECGSSGTCISPSHWCDGILHCPGGEDENQCVRLYGPNFILQVYSAQRKSWYPVCQDDWTENYGRAACQDMGYRNSFFSSQGIADDSGATSFMKLNKSANNMDLYKKLYHSDVCTSNTVVSLRCIECGVSGKMSNRQSRIVGGSSAALGDWPWQVSLHVQGIHICGGSIITPDWIVTAAHCVEEPLNNPKIWTAFAGILRQSFMFYGSGYRVAKVISHPNYDPKTKNNDIALMKLQTPMTFNDKVKPVCLPNPGMMLEPTQSCWISGWGATYEKGKTSEVLNAAMVRLIEPWSCNSKQVYNNLITPAMICAGYLQGSVDSCQGDSGGPLVTLKSSIWWLIGDTSWGSGCAKAYRPGVYANVTLFTDWIYRQMRANS

SEQ ID NO: 440 - protein, 495 aa, Sus scrofa
MALNSGSRPGVGPYYENHGYQPESVYPPQPPGAHRPYGAYPAQYHPPSVPQYAPRVQTHASTPAVVVSRQPKPRSRTMCSSKTKKALCITFALGAILAGAVLATVLLWKFMEKKRCSTPEMECGSSGTCISPSHWCDGILHCPGGEDENQCVRLYGPNFILQVYSAQRKSWYPVCQDDWTENYGRAACQDMGYRNSFFSSQGIADDSGATSFMKLNKSANNMDLYKKLYHSDVCTSNTVVSLRCIECGVSGKMSNRQSRIVGGSSAALGDWPWQVSLHVQGIHICGGSIITPDWIVTAAHCVEEPLNNPKIWTFAGILRQSFMFYGSGYRVAKVISHPNYDPKTKNNDIALMKLQTPMTFNDKVKPVCLPNPGMMLEPTQSCWISGWGATYEKGKTSEVLNAAMVRLIEPWSCNSKQVYNNLITPAMICAGYLQGSVDSCQGDSGGPLVTLKSSIWWLIGDTSWGSGCAKAYRPGVYANVTLFTDWIYRQMRANS

SEQ ID NO: 441 - DNA, 35453 bp, Sus scrofa
  source 1..35453: mol_type = genomic DNA
aaagggcctgtgaatgactccaggtgttgttccagcccctgagatgggtgtcaggttacaagacgcataatcattccagtttggcaacttcacctgtggggctgttttaatcgacctgccccacagtccccaaaccactcctggaagacagatagcagtgggacacggccagctccccagggggagagagatgctggacgcaagggaccgaggcccaccctccacttaagcctcctccgaccagtacaagctgggcatcgctgacctgcacgccagccagggccccattctggggccctgctcctgacttgggctgctgacctcagctccaggctatagaaaattcaggaagatcacagagctggcatggtgagtgtgggaccctctgctccgaagaggtgggaaagggagggtctccccatagaggaaccttcaggcagcctgagaggacatcagctgagaattttgcaacaccatccaggttaaaaaggaggtcacatctgactcgtgcccatactccagacccttatagatgtcggtagccacctgccctccttgccaacagccctagcaccagcaccgcggttgctgagaggcaggggccagggctacactgcctgttctccccacctaaatcctgctactgtcttgcccagtgtttcctggtctgttggctggagactggcaccatttttccaagggcaagaggctattttgacacaaggtggccctctgctacctctcttaggaaactgactccttttctttaaaaaaaaaaaaaaagagagagagagaaagaaaaggagttcctgttgtggctcagtaggttaagaaccagacatagtgtccatgaggacacaggtgtggtccctggcctcactcagtgggctaaggatccggcattgccgtgagctgtgatataggttgcagacccaactcggatcccgagttgctggggctgtggtgtagacaggctgtcgctgtagctctctttctacccctagcctgggaacttccgtatgctgagggtacagccctaaaaagaaaaaaaaaaaaaaaaaggaaaaaaattaaaccatggcaagctgcctcagggggtcatggaaagcacgtctctcctttgggctctgcctcccttccgcctctgatacccagagcttctccactgtcttgggacaaagagaagcaaccccatctctcagagaggacagctgaggacagggaggggagaagaagagagctgggcctgaggcggaacccagggaatcttccttccccaattgctggccaagatgctgcaggtgcccaagaatcaacatgaaaacaaaagaggagggaaggaaggggaggtggggtggaggggacattctcttcagcagggagatgggtgactgctggggcccctctggggatggtctcctgattcccacgtactttctgtgacacttgcctgaacagtctagcaaacctctagctccagggctggggtcagagaggggaaggggctggcctgctttcctctgcaattcagccaggacagagtagcatcagcctcccaggtcccatcccagtgccctggaagcacaaagaagctccatcccgccagggccctcaggaaagaagagggagcagtgccagcctgcctggctgtcagtggcttcttgatggcacttagaacaacacagcagcaggtaatttctagggccaatcactagaacagtctctgtacaatcaaaaggtcacatggccaattcccttgttagcttattagtcttgagactttactctttctgagactcactgtctccctctataaaatagaaacacccaccccaccccctttgcacacacacatagggttggtgggaagatcgaatgggggagtgatatccgtgaatagcctctataaaccagggaaccctggagttcccactgtggctcagtggtaacaaacaagactagtatccatgaggatgcaggtttgatccctgggtttgctcagtaggttaaggatctggcattgtcatgagctgcggtgcagatgcggctcggatatgacattgctgtggctacagctctgatttgtcccctagcctgggaacttccatatgccaaaaatgtggccttaaaaaaaccaaaaaaaaaaaaaaaaaaaaaaaaaagtactaatccagggagcccagtacagctctgtcaggatttgtgactaaaggagaatctgctccagggaggcaggggcgggaggcggccagcactgaccctccactggccagggaaggagccgggcactgctgaatgtgtgagcgagggcaggtaggcacgctggggaggaggagccgggcagcaacacggacacagtctaggctgggacccaaggaacttctggccctggagctaacatttaagccacccgtgcctgacaaccctgttatttctaaagaggcccagacaactacagctcccagccccgggagagatgggacagccctcccctccccttcccctccttgtcccctcccacacctgcgggccactctctggacaaagagtccagcaccgtggctctttcttaataatggttctcctttgactctatcccacgggtgtgcctgggagggctccgggcagtgtctgagcccagtctcccctccagggaaatgtgtcctttgatatctgatacctagagctgagccctggagggaagctggggaccctgggactggccagccttgccttaactcctcactgcccaggaggcccagcaccaccagagtagagtgttcaatgcctgaggtctacccaacccttaggctgtcctggaggaagcaggtcctccccgccctcgcctccttgacctgccagctggtgcagaccaaagggcccccgggcttggccagggctcaggaatatggcaggtcagcaggccagcctgctggccgcgggggtccagaggcatgggcagggtccacctctgacagaggccaagtccaggagtccactcagccctggtggcaccttcctgggttcacagagaaaagagatgagcagtcgaaagacccaagagtacaagacaggcaaagaagggctaggccccctcctgtgctctgggccccctcagaactcactgtgacccccagactagctccattcttccaatcaccattcaatctgcccctcctactggaagggggcacaagagggtacaatgcctaggttctgtcctggaggagctcaacatccagaggcaggacatcttcttaagcaaagaagcctaaggggttgtgaagaatccaacaccagagccaagagaaggcagtaaattccctgtggggtggctggggagttataccagcagagaaagtacttgcgctagaccttgaaggatgagcagccttcctcaggcagagaaggtgggaagggcttcccaggcagaggaaagtgcgtgtgaacagcaagaaatagtgagagtggggaatgagggaagggggctgtggctggagcaacgggcacagagggcacagctgaggcctgagccacatggggctgtcatacaggctctacatggagcctgggttcaaggtcctcaccctgctagggagctcaggggggccaggcctggtacagctcgccatacccctgaggcagagctgtagggatccagctccttttattcattcaccccaagaaacgggcagcacgcctcctctgtgtccacactagatgtgccagggtgagaaagcagatggggggcctgtgcacgtggagctcagagactagaggaagtggagggcgggaggggtagatggttatcaaacacacaaacccatgtttcagtaacaactgagttacggggctgtgatgaaacacggagggcactgtgagagccggaccagggtgtagccttggtggaggtcagcggaggctgaggactgcacaggagctgggatgcagggagaagggccaggccaggtggaggaaccgtatgtgagaaggcagatgtgcatcccccaggccctcttccagggctcttcccctgcccttggggtagaggtctgtgcctcctgctttggagaagtggcttaggagccaggccccgtccatgccctcaagtgcttcctcaacctctccaccttccctccccacagccaggaacccagaggttgtctgtaacaaacgcccttctccccccagcctgtccccttgacaaagaatctcaagagctgaaagtggggacgtgaggagagaggctagtccagggcagagggggaacacagcagaaagagatcaaaaatggctcagggactctgcacagactttccaaagccctggaaccaacccaggatggccagagacaaatggtctcaggtctgaaactccctcagcgggcctctcagatttgcaggcagctctcctcccatcaggctgttttcttgctcagatgaaaacatttgcctctatgaaacacctcaaatgcccaagccatgatttctcaagacaatgattccctaaaaccgaaaatgcgttcatctgccacattcccttataaatctgttgctaccagactgtgagcttcttaaggaaggtctgagtcgctatttttttagcctcaaacctacagcacagggcctggcagagagcaggcacttgatgcatgtttgtgaatgaaggacaaataaattgaccaaagactgaaacccctagaatgagtgcctctgatttatctcaccgaaaacaagattagccaagtccgattggaaaagatgccagatgctgccggaagtgttacccagcaggattcatcccagatggggccggggtgagggaggggcccgcctgagtccaaccagaaacccccacgatggggaggagagagaatctgagcaaagccaatgtggcaatccccctgcaattgtgctcagctccacctcgaagcctgcagccctaactgttgcaattgcagcagagaaatttgtcaacaagcctcaaaaccgcaagacgtcgccccttcctctcccgcccagagcagctgccaacttcctggcataatccacaagcaaccggaaactctcacagggccacgggtgaaagaacactggaggaagaggctgagagactcagatggcacctgtccaggggcggcagcagtgtcttctgtcacaaaatgttgtgcacatccatctggccgggctctgcctgctggcgtgcctgagcccacaaatgctttcagcatttcctgcagtctcagtttcttccttgagaatgtgcattcattcattcggcgcgttttcctgagcatacaccacatgctccgcgctggcaagacatagcagatcagagaacaccccaagctgaggttttgggtatactccatggatggaagagaggcaggggaaactacttcccccaccccgggcagagcagatgtgacagaaagggagtgacgaagctgaggtcaccttgcccaccttcatccttttcccacccaccctgccactggggacttgacgcatgatcacacctctgacccacagagaagagggtcctgccaggcagtcgggaggacctacctggtgagcaggaaccactggcaccctaaaaagaataataccagcagtcacctctcatctctacccaaacacagaaagggcagcattttgtggcttgcaaagcacattcacagccagaatctccctgagtcttagctcctgcttgcctcccaccccctgagcaacctctgggggctccgccaaatgccccaggaggccgcagagccctgagctggccttgcttttgtctactgtggaaatttccttcactgcccagcttctactcaaagaccttctgctgcttcaaacaacttcccctttgccaagagaaagccatctttctggaacctccccgttcatcttcagcatcctctttgcctctaccttgaggtggccttacacattttaaaggaccatgtctgtgccaccctgagtcttgatgtctctggcctgcatcctccaccctttgttcatagagagatcatacaggtcacatgacaccatttcaggtgccctcaatgccctgctcactctccgccagggcaccctagtcctccaattctgctgaagtcagaacccttgccctaatgaaaccactaaaatagctaccctttgctgagctcctattacacacatgtcaggcctctactaggctcttcctcattaataattaatgatttttcagtatctattatgtcaagcacttgctaggttctacataaatattgttattaattattaataaaaaataagacaatatatattcctatgggcacataagcctttgcctccttgttcattgctgaaacttcagccccggaacaatgccttactaaatggataaagcagtatcattactgctgttgttgttattaatatttattgcattcgtactatatactaggcaatgtgctatatgctatgcatatttgggtcttaagaggaaaactttttttttctttttttggccacccggaggcatgaggagttcccaggccagggatcagatctgagccctagctgcaacctaagcggccacttcggcaatgtgggatccttaacccactgtgccaggccgggaattgaatttatgacccagcactcccaagatgctgccaatcccattgcaccgcatcgggaactccaaaggaagacttattgactcaccctctctgtctttctcccatacacacacacacacaaacacgcacaactgaaactcccaggggttaaataacttgcctgaagtctgtgctccatccgctataaaacacaacctcccctagcaaatggcagcccagcaagaagaaacttcaccgagcgctcccacagttatgtgagtgggcgggaaagggggaaaggcgcttcaggttatgcaactgcaaagtaacaggggcagagcagtcagcgcagcagcaccaaagcactatttataggatcacgctttcagggttcaccggcagagatccgggaatcactacagactttggccaggagatggcagctcttcgttgcagcagccaaaccggcccaccggaggctggttctctggcagcatctagaacaagggagaaggctcagtcctgccctgtgcagagccgacacgcagaccctcctccagtgctgtggggagtcctgctcacaagcctccagggcattggagccaagggaaaaagactcctctgtaagtcaaagcactgagccccgtgcatcttctcagttcatcctcaccgacaaccctgtgaaactgatcacattctcccgtttcacaggtgaggaaactgagactcaggcaaacgaacttgcccaggaccaggctcagtccagagctgactcagagctcaggaggttacagaatcagggacagctcccccctccccacgactaaagtgattgcgcccccctcagactgcaaacaccaaccacagagaaccatgagcaagtctatgatgatgatgagaggaccagatgggcttaaaaggtgcatcataactagcaggtgacaaccctctcagtctgatcacacctgagttccaaaggaggcaaccgggcccaggagatgttaaatgattcacctaaggtgaacagtagctcaaaaaatagcgcagcaggtaaacaggaggggcagtctttatcacaccaaagtccaccttccctccctgccatttctctccctgttaaggtttaaaaggacaattcagaacaggcaaaagataatgtggtttttcaaaggcaatataaactttatagggttcttttataaacctcagccagcatccggctgttctatagttcaggtcaattgcaagatttagggagtcctgccaagctttaagatccattagcccttcccaaaggcaaagccctaagggtccctgccagtccaagtggcctgactccggagggctctcatgtatagcttcgtctcccgatccccagccctcacttcatgtcccctctggcatcacagccaggatcttctcaggaaactcatagcttcatctcccgatcccccaaccctcacttcatgtcccctctccaaaccttacctgtcatagctgaggcaccccagaggcagggtaccctttccggcttggcaaaggcatcaaagtgcaggtggcgaagacacagctgcttgccgttaggctgggggtgggatgcaggttcaggggtcagagaacagggcacctgagcgagccaatggtcaaggccactctgtgagctgactctgcacagcttctgggtcccctggctcactcaaagaactttcccttccccttcccctgggatccccccaccccagcactgtgtcatcagccttagcaacttagctgccacctgccagagccacagaggacctgtatattccatcgcctggcaacagcagcatccctgctgtaggtgttccccgagagaccaagggaagctggggctttagaccccatgggagcttaaagctgccacaaatcagatcagtgccagaaagcccgttcctctaggcccctgcttttctcccttcttctacaccccaaatcaccccagttctgcccctaccgccttctttccaaaacaaggaaatgcagagtcggctttatggaaaggaggagagaaataatgcccaggggcaatctgcttggcaaggaaagcagacagaggaactggccaggctttttgttcctttggcctctgtgaactgttgtctctctttgcctcatcattagcctggtggtgcctgtttcggtgtgttgtgagtaacactcttatctgcttcaccatctctgagttcccaattacccaaaggaacctcgaggttcagagaggttgaggagcaggcctaaggccgcccagcaaatccaaagaagggcaagacgagaactcatactcctccctggctgggtctctgtggcttctctacagtacacttaatcctcaccccaaaacaggagctgccttgtgtttgggttcatcatctctgagagcgtcgtctgtgatttggagctgagctaaacctaaccaggagtttctccaggatcaaaaacaacaggggctaattgggaaataaaggaaggagagaagcaggtgagaaaggtgccacgccaatggaggagaggaaaggagagaactaatatcaataaagcccctactaggcaccaggccctgcaataggccctttcctcaatacaatctgggaggaagttctccttaccccagttcatggatgagctagcagagtcaggaaccaggggaggtgggtgtgctttcagcctgagacatgctgggcatcatctctgccgttgattgcttgctaattatatgcaaccagacttccttgcaagggagtaaagaatcagaaggacccagcaccagctgatgactcccggagaggttcctgaaatggtggggaaattaaaataatggtccaccgagtccaaaacttgtccccaagggctggatttgttacttgctctgaggccatagcgaccactgcatctacaggagtcttggtttttctcttctgtgaaacggggatcatttctcagagttgttgtgagggtatttaatcctcacagcatttatgaaagagatacaacaatacccaactgcagaattaattgtctgggtcttggattaatgtctttctgcctccagtgaccaactgagaagcagagaaggaagattctctgagcaaatcaggtgatgggcgggtgtttttcagtcctctaatttccacccacccctgtctcctccagctcgggcccttctgggtattcctgtcctcctactgcactccagacagcaggggcctccttcccataggatcctggggtcagacactggttgggctggacccgtgagtcagggctctgcagcagcctggatcagcagagcagccaggcctgacccagccaatgcccccaaacgaaaggggaaggaccgtgccttggcgtgggctgggcagggcctggggctgtggcagttgcaattgcagcatttgaacaggggataaacagagacgcctccagcaaagcagaaggaagcacagctggtctttggtggactctcagccccttccctctatcaattccagggcaagacacttcctgtctggccggttttctcatcagaaaacagtgacgttgcctgtgatggtctccagagcccttggcatcctgtgatcatgcagggactcactatagtgtggggggcgggctggatcccgaggacttacctggttcccacatctctcagccccccttgcctcctttctccagctcaggattgattctggactgaaaggcaaggcagctgccctgagtcttcatcgccatccttctcaggaaagtcattgacatggatgttccttagctgccactcaactaaaacgcacccacctccctgcccattatccccgatacccctgaccttcagaagttccccttcctagcagtttagcaacccatcacctctgatctcttcgtgctggagatgtttgccagagaggcttcaagaaaagccacagagtgagccactagatggaagaacttgggcagcataagagtcagcagaaccggccagcctgcctttttatcttctttttgaccctcaggtacacggccctgggtaaatcataatctttgatgcatggcactgttcactgttggtccatgcagggctccctgcaactcaccacctagttccatggtttcagcaatgaaataagcctccaggagttcccactgtggccaagtgggttaaggatccagggttgtcactgcagcggctcagtgctgctgatgtgcaatgggttcaatccctggcctgggaactttcacatgccatggggtgcagccaaaaaacaaccaaccaaccaaaacaatgtaataagcctccagagagccaccacccaaaacaaaagctaggatttaacaataacctacatctaaccgggggagccccctgctgggggaggttggcaagtcttttttgtttactagtttaacaaatcttgattgtacattttgatagaccagagacagtgctagaatcatcctgaatcccatgctcatcatttccttattctttatgaaaaagattctgtgccatatgaaaacttttgggacttaatttttcctttaatttgaaattgcttaggctattcatattactttgtggcaccatagatcatgtgttttgagtgtagcataacattccgttatgtgtctataccacagtcagtcagcctgagactgagcctgtgatattttcctcaatgctttcattcaataaattttgattaaatacctgctattataggagataccaggaatgcagtaatgaccaagataatcacagcctctataattttagctttcagggtttttttcctctttctcttttgttttgaaaagctatggatttttctccctataattaaccactatgcagttgatagcgattgaaaaacgtattagccaaggagttcccatcccggctcagcggtaacgaacatgactagtatccatgaggactggggttcaatccctggccttgctcagtgggttaaggatccagcgttgctgtgagctgtagtgtaggtcacagacactgctcagatcctctgttgctgtggctgtggtgtaggcgggcagctgtagctctgattagacccctagcctgggaatttccatatgccatgggtgaggccctaaaaagaccgggaaaaaaaaaaaaaaaaaaaggaagaaagaaacaagggtactaaggtcactttagttcagagatgaggaattcaagaaaatagaccaaagcctcttttgtttcctctctcggcttctagcacaattccacaggcatcaaagacatacagtcaatatttgttgaaatagtaaaccaaagaaacttgccctgtctcctttaaccaatgcaggcatgtgtgtgcaggcagactccatgaaaaggaatgatttgatgggggaagggccaccgattggatgtccctgatacccctccaacctgcccctcctgcagccttcggcatcccagcagataccctctcacctgctcactaaaaacccaagagcatccagtttgttggcaaagcctattgcctttatcatccacacacaggtggaatctttgcaccagccgtcatttctccctccctgctgtagccacgctgagctccattctgtttctccaacgccattctgactcctgcctgcctggctgcttcctgtcactcaagtctcaggccagaggccctccctaacctgaccccttcctagacctcccactctatgaatccagccaggcgctgtctcctgttatcctgttactctctgtctagccccatcactctctgggtttatctgttgacgtccgtcttcctctattctcccccgcaacccccacctcccccaccccagggatacaagcttcttgaggccatggtgtcacctaacttctttgactcttctcccagcctgctatttctcatttgtctttgaatccacaactcctagaacagttcctggtacaaggcagacgctccacaagtatttgcttaatacttagacctaggggatgctacccactctgcttattagacaagttatcaattcttttcacttttgaattcgagttcggcaaaaggactgtgtaaactgctatgtgctgactgtgtatagatgactgtgggtaagattctcacccaaatctgtagctaccgcaatgggaacctgcaagtagttccgggccctgggaatggtggaggagagacggccagggaggaactacagtgcagagccaggttcagtttagtttatcagtcctttactgagcctctgctaggtacgaggtggatgcaaagcactgaggacacaaaggcaataagaaatagaggagttcccgtcatggcacagcagaaacaatccgagtaggaaccgtgaggttgcgggttcgatccctggcctcgctcagtggattaaggatacaacgttgccgagagctgtggtgtaggtcgtagacacggctcggatctggtgtggctgtggctgtagtataggccggcagctatagctccaattagacccctagcctgggaacctccacaggccgcaggtgcaaccctaaaaagacaaaggaccaaaaaacaaacaaacaaacaaaaaaaaacacctaaaaagagaagaagaaatagagaccactgagcacttagtgtcatcgggccttttaatggagataaatagagagagctcattgtggtttaaatttatactggcctgatgaatgatgaaatcaggtatcttctggcatttattggccatggaagcaccccttttgcgaagtgtccattcaaatttttacagaactacaacaccacctttaaaagttgacaaatgattgccaaaaacaccccatggatcagaatcatcttaagaaactcagcgatacccacaacagcccagctgccagtcattcttacaattgctaactagttactgcagctagtaaccctgttgtgccgggtcgttgagatgcataactgaattttctattttgaaccaattatttagacatcatagagtcccatcccacccccagcttagactcagcagggagagggaaagccagataagagccccgccctgagctcctagccccagcctgagaggctccctcactccactcactgtttttctttcaattacaggatcccgccagtgatcaacccctgaacagccttggtaagctcaggtccggctttcctctgtttacctcagcccagtctgagtttcaatcagttactcactcctgggggtgaggagtaggtactaaactcctcacccccacctccaagtgctaaatgctatgggtgctagaagagcataggtcctactgtgcagcacaggaaactatattcagtaccctgtcatagaccataacggaaaagactatgaaaaagaatatatatatatacacatatgtgtgtgtgtataaaactgaatcacttagctatataccagaaactaatacaacattgtaaatcactatgtttcaattaaaaataataataaaaataagtaggaagttcccttgtggcacagtgggttaaggacctggcattcccccagctgtggcacaggtcacaactgtggcacagttttgatccctggcctgggaacttccacatgccatgcgtgcagccaaaaaaaaaaaaaaaaaatcgtaagtgggggaaaaagtaggaaccttaaaaagtaccttaaaaatgctatgagatacattcatccatcattcaacacatatttattgagcagtgtgtgtaagctccgggattcgctctagaagtggaaggcagggtctctggggaaacaggaccatcaaacctaaaaccccaacagcagtgattctctaatccatttatcgagttatccactctgtcattcactcaacaatactgtgagggcctgccatgggccaggccctgggctggacatggggctgcagcagtaagtacaacagtccaagcccctgccctcgaggagtttcccgcactgaccaggctgccaaggaacgagctggacaaaggcgttttcactggtagcatagaaaatggtagtgtttgcccgatttacacaagaaacataagttaataaagtattcccaaatgcagggtgtcttcccttcaagcattttcttaagccattgataaccaacacctgatagcccggctccgcaaaacagtgagcaaagaacaggttgctcactgacaggttgataaggaaaccaccgacctcacttacgttgacagcttattcatttcagggtcacaagttaattaggatcacaaacaatataatgtccagcaggtaaaacgatgcctagtttatactttggaattgaatatgtcactcttccgtggtgtccccaaagaataccagacatacaagtgctctacccctgagtaaacctgggacctgccaaacaggaaaacaactaaggcctctccggagggaggggtcagtgccacgtgatcaggaaaaacaggggggtcaggcacgctttgacaaagcccccccccacccccaccccccgggagaactgccacaaagcagaaggaagtgaggagggtctcagtggtggagggaagagcaagagcaaagattcccaggttaagaatgatcatgggttgagcaaggcctggggagtgaacctgcccgcgtgcaactgaggctggcgggcaccagcgatggcaagtccatttggctaggagagggaggccccatcacactggctttgaaagcctaggaaagagtgtggtccagatacggaccatggggcgcccttgaggattcaggagcgagaacgtgatgggatgaaagcagtgtgttgggaagctttgtccagcaggacaggctggaggaaggaccaggggcgggcagggagcccacgcagagctcagcgggctgttaactttccgtgggaaacaacaaaccaggaaaacctttcccttcatggtgaacgctctccctgagaacctctggggcgcagagacattgggtcccggacgggggaacttcatccagggactcccgataagtcattcgcttctgagaaaacaagccaaccttgtacactgcccccaaagcaggtgctcggtgaacagatcgagcatgaacgaatgaacagaatgtctccctgtccacagccacagcaaagaggacagctctgaacactcacggctgacactttaaaaacagcagacaccaacattattaacttacgctgtcgccaagtcctgataagagcaggcaacccaagcccacatccccagggcctgagctccgtgtgggaagtttcggctctcccttttgaggaaatagactgtcccgagcttcccatcaacagacccaatgagatcagcacttaccgcgcaccaggctccagcaaggtctcattctctgcttttgaagtgctgagaaaagatgtagagacggacacacaaccagccttcactcccggcagaataaaatgaggcctgtaagaaagatagaaggggtgtgctgaagagaacaagcaaagaataattcatttattctttcaacccatgtttattgagagcctactgcagtacggaccaggcactgttctaggcaccagggactcagcagtgagcttaagacagataaaatatccccacattcatggagacttcatctcaggtagggatgggaaaggataggtgatatgcaagatttgtaaaatatggagtacattagaggtgacctatgctctgcaagaaatgaaagagggaagggggatagaaaatgctggaagacggagttcccattgcagctcagcagtaacgaacccaactagtatccatgaggatgtgggttggatccctggcctcactcagtgggttaaggatctggcattgccgtgagccgctgtgtaggtcacagacccaactcggatctggcattgctatggctctagcggaggcgggcagctgcagctccagttcagctcctagcctgggaacctccatatgcctcaggtgtggccctaaaaagcaaaaaaaaaagaaagagagaggaaaaagaaaatgttggaaaactgcattttaagtagggtggtgaaggaaggcctcagtaaggaagtgccatttgaagaacaatgtgatgcaggtcagggagcaagccggggtgcaggtgaggggaagggagctggcctactggtctccagggatattcagggtcattataattcctgccttggttctccatggagaagtccccgtggcaccttgccagattattgcctgcaaggagaggggaccccaaagcactgacctctgatactttaggagaatttaactttccaaagccttgctgtgttccatctccttggatagccacatgaaacagacagcagagctattattacctcgggtttagacataggcccctctcagtctccctggccccaggccctttcattccagtcccttgccaccctgatgagggcgtcctcgtcctcaatagtaatgtgattgtgtctatctgctgctcaaaagcttcccatggctcactggctgtaggataaaatccaaacccctgaggagttcctgctgtggtgcagtggttgaggagcgagcattgtctctgtgtcaggcgctggtgattgctgaggcgcagggtggatccccagcccagcacagtgggttaaggatctggttgcagcttggatccgctccctggcccaggagcttccatatgcttcggatgcagccagaaaacaaaaacaaacaaatccaaacctttagcatggtggtccagactctacaaactgccagccctgcccccacgacatgcttcacccaccttccgtcgcagacacaccagcaactcacagctccccacgcccaggcttcacccagttgggccagctcctccctctcccgtcgccacctacagaggtacatttaagtgccttcatggtgtctggttgtactggcaactgtgtccttgtttgcctcttccctcaaagtgtaatcgcctccgaagacaggactggctgatgctgaactcacttggagatggaggctgtcaggacacctttgttcactaaggtttattaagcttctaccaagcacagcctgtgctggcagcccctgccctcctgaagcttatagtttaatcaaaatacaagtcaagaaagaggataaaagacagtaaaagggaaagggtccgtgcctctttcagagcagacgcaggtcagactggacagggcgaaacatcctagaacaggctgtcaagacaggggaaggcgccatgggctcgagggacaccagtgagagatggacaagcctgggcaagtgaaaggcagtggcagggcctggggatgcacatgccaagaaagctaagcctagactgcaggaataaccccccagcaccaaccaaaactgctcccaaagtttcctcaaggctcagcaccacaggccagagtcaggggaggctgtgggccttggcaggggtggtgtccgagtactgactgtaaactcttgccccccccacgtcacagatgtcgccccccagcgcaaaccccgcacccccctgcagaccttcaaaaaggtggggatccccatcatcgcggcactgctgggcctggcagccatcgtcgtcgtggctattctcagtaagtagcagccccactccccaccccccacggcacgtggcagctcccacccacccctccccctctgccgcctcagagcacctccccaccactcccaccccagcagagtcaagggcaggtgatctgcatcccgctctgtatccacccctgctctgaagggggacttcctctgagagagaacaggaggagaactcagccccacccatcgcaggcggcttctgtcccaggtgatttctggggagagcagtttgcactgtgtccaaacccacacaggcaatttgcaagcagccgctcctgctgcctcctcctcccaagtctgcaggctgagagctgagaggatgctggcagccctgcaggctggagaagcaggggcatggctgcccccagggcagggtcagagatgaggcaggaggaagacggccaggcagacagcgcccctccccgcctttagctctgtcttccccagggcctgagactctgtgtgatcctgggccagcccctctgagacaaagaagagtaaagtgaggagttcccattgtggcgcttcggaaacaagtccgactaggaaccatgtggttgctggttcgatccctggcctggcgcagtgggttaaggatccggcgttgctgtggctgtggtgtaggccagcagctgcagctccaattagacccttagcctgggaacctccatatccaggtgcggacctaaaaactaaaaaacaaacaaaaaaaaagtgagtgagccctgactcccacaatccctctctttcagtccccacatcctgagactgagtcctcaccttcacctccagagtcactgcaggcaactgaccacagctttcacccagggagtagggcaaaggggaccagactaggaatcgggatacctgggacggggatcccagcactgccccttgctgctgtgtgaccttaattaagtccctcaacctctcttaacctccatcttctcacttgtaaaacattctgcctccctcagagggatattagagaaccaatgaggtaagatgtgcaaagttcctggataaagatcaagttgtgcaaaagtggaggtggtgacacagcatgtagggatgtttgcacaccaggccaggacggccttccaggagccctcggcaccctccgcctcagccaagatgctcaaagctcacctggtccaaccctcactctccacttgagtgctgaggcagagaaaggaggcgtggcagtcgggcgcgtccatttccacctgctggtgatggagatgcggatccagaacccaaacaccacccagatccctgagcgccccactggggtgtgagatgcagataaccccaaatcattcctcttgaccttggctggcccacaggggtgatgggtgtacagcggtgtcagcagatttgtcatcctaagcccgttttgctcagactaaaattcagatgagcttgcctttttgaagcacgtgtcttgccatagtggcgagagaaaatgggaccaagttcagggtgagccaggacaggtaccagcacataatttagagctgggctctccctggcctagaaggcgccacgtggcttccagcctggggcctctagtttctgcaaccagaagagggaacgtgaacttccatcctggcctggcctccctcaaccatctgacttctgcaagctcaaccacaagtgaccttgtgtcttggcttcaccccaggacgcagaagtgacccttcctcaccctgcctcctgaccacgaaccaaactctgaaagtctggcaaacatccggcctgctcccttctccctggcttctcaaatcaggaacataagggccagcaggtcacatcctgagctcccccagggaaggcatggagaggccacaaagagctttggagccagacagactccaactaaaatcctggttctgctgcttgagtgctgtgtggccttgggaaagaaaacctagctctccaagcctcagcttccttctttgcaatgtgcgttcctcctggtccctgcctcctaaggttgtcaggattccatgcagcactgcgtggaaagctcttagcagtgataagcagggctgtcatcagacttgttaccgcagctcttagggccagccagggatgacgctgggctgcggaggggagtgacaagctaagggagagttctcttgggcaggaagagtcaggacagtttagaaaagtgttgaaatatctgttcccttgggatttaaaaatactctctcattcccatcatctaagacagcaaaggtgcaatagtagctgcaggcaggaggcaggaggcagaaggcaaaacgaagcaaaactccaacttcttaatggggctttcactcccagataaaaacatctaactccagaactgcacttgccagctctgagaaactttctcaaccaccagctcaccaaattttctccacaaccccgtgaggcccagagaggggaagcaacgggtctaaagtcacacagcaggtggcagagcccgtgctcaggtctttggactccaggcccagggttgtctcctctgtccccagacacacttggggttaagaagctagtgtttctaaatcgctccccacacattatcttgattgactcctcagccacctttagggatgggacaggtgttattatgctatgttacagatgaggaaactgaggctcagaaccacgtacaccagctattaagtggcagaactgggcctaaaacccacatttgcctgtccccaggcccagggtgttctcagggaaacacaacctctcctgcccttggccacctgcttgaccatcaaccgtctctgtcctgccccgcccttgccttccagtcaaggtgattctggacaaatactacttcctctgctggcagcccctccacttcatcccgagagagcagctgtgtgacggccagcaggactgtgcctcaggggaggatgagcagctctgtgtcaagaaattccccgatgggcccccagtggcaggtgagtccaggggcagccgggccaggggagtgggcacagcaggaagtcggcccaggtccccttggttctctgtatagtacctgccacctgactagtcagtttccgtccctgttggaaggttcttaaaaacaggatcacaggagttcccgtcgtagtgcagtggttaacgaatccgactaggaaccatgaggttgcgggtttggtccctgcccttgctcagtgggttaaggatccggcattgtcgtgagctgtggtgtaggttgcagacaaagctcagatcctgcattgctgtggctctggtgtaggccagggctacagctctgattcgacccctagcctgggaacctccacatgccacgggagtggcccaagaaatggcaaaaaaaaagacacacacacacacacaaaaacaacaacaggatcacagcttgtctagtctactagagctccagtccttaacatgttgcaaacactcagtaaattctgatcaacaaatgttggctgggggacaggaaattgtaaccgagcctgctccattcctgctgtacctggatgaccttggggagtgccctcctttctgtggaatctattactgcaaggaccacaaagcctttaaccaactatggatctttctattcattaaataagtgtttattataacatgttgtgtaataagactgagttgggtctttcaataggatattgagatgtttcaaacgtaaaaagccagtttccttcttggaccgggctatcccagcttgatccacccacctagtagttgggctctgaaatgaaaagccagcctaggacatgggtgatgagggcttttgtggggcagggctgagtggtttcctgtgtattttctctttttttgatcagaagtcaagtgcctatggaccctgctaccctatttccaatagtatttctttttttttttttttttttttttttttagggccgcaccctcagcatatggaggttcccaggctaggggtcgataagagctagagctgctggcctacaccacagccacagcaatgcaggatccaagccacgtctgcgacctataccacagctcatggcaacgccaggtccttaacccactgagcaaggccagagatggaacccaaatcctcatggatgctagttgggttcgttaaccactgagccacgaggggaactcctccgatagtatttctcgctctgaccttccatggtgaaggaactaggtccctcattgagaagttttccctgactaggcaagccgtgtagaccaggggtagggcagccatgtgatacatcatcaaaacgtatacatgagacaagcaaaaaggggagctattaataattgtgccagggccaaggtcaaaatggagattgtccccaggacatccacattagcagccttcagaatgaggctcagtcaagcaaagctcaatggaggaacttcccttgaggttgaaagggctggtgttccatgatgttcaccagttccggggagggggagcttgggctggtcacatgatgagttctgtggaaggtgcatcagatgggtccctgccctgagctgacccaggtccctcctctcccagtccgcctctccagggaccgatctaccctgcaggtgctgcacccagccacacagacctgggcctctgcctgtttcgacaacttcacggaagctctggccaagacagcctgtgggcagatgggctatgacaggtacccagcccaaggctctggaggctgttatctcactcctccttctctctctcctccttcctttccccctttcctcctttccatccctccttcctccctctttcctaaaagtacagatattagggtcagacagaatggcttggaagccctttcccttataagcagcatgaacttaaatctgcttagcctcggtttgttcaagagctaaggggggagttccccttggggctcagcgggttaagaatcccacaagtagccaagagttaatgaggaaaacatgacaacctcataggatggttataatatactagggagagtctctcgcccctccccaccccacccccacccacagttccagcccctttgcaatccttcccttttctttcctcctcaatcctcactcattcttggctttgcaagacaccgtccttgccccagaatacaaaagtcttatttacttcttcaaagtcccattggttaaaatttagttcaagtgaatctgatatcattggggcttcttcagcagttgagctctctcagcaaatttctaaagcaattgtaaatctttggtggatgaaaaagtatataattataagaagggtaattagcattacaaattgatattcatttgatcttggcaaaagccaaaaaggtagttattattctattttagaaggaggttcagagaaggtaagtaatttgcttaggtagtagctgggagcaagagggctggaattcatgagcaaggccttctaattcctgggtatcagaagttttagaagcgtctctgtttctaggttgggtaattctctcttctctggaggtttcatctataaggggttgggggggagacagttgcccactcagagcctaactgaagaggatggaaaaatacccatatattcagttgcagaaggataatctcttcctggcaggccccaaacttcaggattctaaactggacccggagagtaggcagaaatgatcaattctaggcagacatcatccatccatcacagaacccttcccatcaatccctaaagcaaggtcccttttcaataagtgttcggaacagccattattgatccatcagacctatttgccccttctgctctctggttatcaggcttctgcagctctctgccgtccaccggggtgcaattcccagtaccctctggagaggacagcctgcccccctccattttccaggagagctgggaaggcatgccctgcccctgccagctcccctcccccagcagccaccccagcccagcctcctccccacagccggccctgcctccatactgcagagcagccctgcagcgccagctggacccagcctagcaggctgcagcttggttacgaaaccagggtgtctccgggggttcttaaggtaaaaggctcctggaatctctgtccttctggagttctttaaggcaccaagccacctcgctcttgagtctgcaagacgagagctggtcagaactactcatcaggatgtaactctgaacggacactaactcccctgggaccgaccatttcccacatagtccttccgtctgggtccctgatttcctggaaaggaggcccttcttgtcccaggaccacaaaaccctagatggttcaaggtgggaaggactctagagatgcatctgatcattttatagaggggaaaactgaggcttagagaaacccagagacagaatgtgaccttccaagggtacacaccaagctaatatcagggccgggacaagaaccttggtctcaattgagggcacgcagcacaaaatccagcactgctagactcccatcctccaggtgccgtgcctggatcatccaggaagtaccagattgccggctggggaggtgagaatgtgccaggcagctggtgcctcagggtgacccaagtcctgcacacagagcttgtactagcatggctggatgggggaggacaggagaatggccaagaggcagattcagtacaggaacaatcagaaagatgggattaatcagggaagccttcctggaagagggaagacatgcccaaggtttgaaggaagacaaacgtggattattggagagagagagggagagtggtcaaaatgagagaaatgcttgtctagatgcatgtgaagtttgatcttggcctgtcctatctccaaaaggaccataaatgtagctcaccaccaaattcagtgcagttgccttgtttggagaaccaaatatccacagccagttcactccaaggtgagggggaagctaagctgcctcctttgctgccttgtcccctcatcaggaaccagaagaaaccgctgggacatttggaatagggacagagctgggagttacgaaaagccatctgtaacatttctccagagctctgagtttgggaccaggcttccctgcccaagaaagcaaggccacatccacactatgcccaggactcagccccgggatgccaagggatgtcatctagcaaggcagacaccaccctcactaaccaagaccatgtccatgccccagccgtgccccacccggcctccactgccacccacgctcttgggactgggtgccactcccaccaacgtgttcttaactcctgtcccacctcactcctctctcccccccctcctcctctcgctccccctcaatctctctcgatctctctctcacaacacgagcagcaaacccaccttcagagctgtgaggattggcccagaccaagatctggatgttgtgaaaatcacagagaatgaccaggagcttcgggtgcaaaactcaagcgggtaagtgagaggacacgcctggcccacagaggtgcacacgtgggccctgctatgagggctgcggccagcccatgctggacagacacagcaagggagacataagccaaacaaggggaagaactggccaggcagactgtcatccaccaagagctagagcatcctatttctaagaggttttctgagtagactctttgtgttcatacaagaggcagccggacaaagcatgctgtccacatgtcggtggcctttcggtcaaaaaggaaaagcacatttgtttcctacagccttatattccctataaagcccaacaggcaagattccaaaggccactttgtccttcctgccactcacccaggccagagtaagccctcccacatttccagctctccagaggacaaaccgcatggcctttgtcaatggcctgtccccatgcctaccacctcgcagctgagcctggcacctaaaggaaaactgtcaccctgcagcagagtgactcagagagaatgcaggtttcttagcattcagggaggctgccgagggctgcttcttcaccaaagagtaagtcttgaaaaggacaggactgagagcaaaatgatcgtttgcctggaatttcatcgaaaacgcattcctcttctgcgtggagagcccacagtgcaacagacactgtcccagcctctgctctcttctgtctggggctgccaagagcccaggctgggctgtgactggggcagggccaagcacatagcagcggggatggaatcgcatcgaattaaatcacactgaactgggtggtagcgcaatgcctgtaacagcccccagcatgcaggttccatatttcccgggtactgggaggggacctgaatcaatggtgcgaggggaactggggtccctgcctcccagccaagggacaagcagaaaccacaggccgcctctgtctcccttccagcccctgtctctcaggctccctggtttcgctgcactgccttggtgagtacactccatctccaagggtttgggtcctgggccagcaataagcagggaggaagaccttccccctcagtccctaaatctctcagattcctggtgtcactctgttttgtcccaactttcctcccttggccttgagcttgtaggtcaaaactccttcaaggggttgatgttaagaagtccctcccagagttcccgtcgtggcgcagtggttaacgaatccgactaggaaccatgaagttgcgggttcggtccctgcccttgctcagtgggttaacgatcaggcgttgccgtgagctgtgatgtaggttgcagacacggctccagtcccgagttgctgtggctctggtgtaggccggcggctacagctctgatttgacccctagcctgggaacctccatatgccgcaggagcggcccaagaaatagcaaaaagacaaaaaaaaaaaaaaaaaaaaaaaaagtccctcccactgtccacacactgccccacaaccaaggtggggctccaggagggagggtaggaaagccagaaagggcaagactgggcccccaccccctcccacctcgggtcacaatcagctcatctatccacaatctgatcacccacatcctgcttagcaaagatgcatcaaacagttacaatgtgtcagcaccagagtttcctgggggcttagcagttaaggatccagtgttgtcactgccatgtcacaggttcaatccctggcccaggaacttccacatgctgtgggcatggccaaaaaaatatatgaaataagaatgtgttaggactgccaatgatgagtagaagatcccagacaagtaagagaattggtccctgtcttcaggaactagtggtgaagcaaagacatgcttcagagagagggacacagggggtcaggaatcctgggcatcagtcccactggcttccagtgtgaccttggataattcagtccaaatgccccagaggatcctgggagaccccctggctctgagaggcgatcagaaggaaaaacacacactgtggccccactcattccaccagcctggtgggagaaccgagcagagcctggcaggatggccagaaaccgggcaacctgagagaaggaaggtcatcgtggctccctctgcagttgccctgttgggataagactcttagtcattatcatgtgacccccgcaatacctagtgagtgggggctgacactggtggctggtcagtgaatgaatggtcacatcacctgatgtgctgtgctggacggcaccgaagacacaaagaggcttaagccccagaccctcccttgaggagccttcgggctccctgagaagacaagaccaaaccacaagggcatggaaggcaggccaacgcccaggcagcctgtggaaatgacgagagagggaaatctccacggactgggcggcccggaggtgggggccaggatgaacatctcagaatgaactgcaggctctgggaggggagggaccacgtctgcctctccgggctgtccacccgtctcctcccagaaccccagagacacagtaggcgcacaactcaaatctgctgagtgatcagtaattttgataaacaagagaggagacagcatctccgggacagggccttcctggagcaaaaatagaaagattttttagggagctgcctgcagatcgatttggctggacagcaactttatataaggacagcaggtggctgccaggagggagagtttcgactctaccatatagacaaagggggtgcggaggggtgtgagtgggggagggaggtgacaggtagaaagtgaatgtgatggtgtttgaaaagctcctgccaggcatggtcccagagagggtggtagcctttcccccttaaaaccaccactgtggccgccactgacaggctcggagaggctgagaacagagccgggagggggaactggagatgtttatacacttgcaagtcagatgtaggtgccaggagacggggatgtggaagagggcacaggatgtcggattgggagactctctggatggggggaagggacttggagaaaggtgaattcgggatgggaagatccatatctggaactgtcttcccactctccaggcccaaagcagcctgtgggcgtctgtttactgctacgcagcgggagcagaattctgtgtccctgttgagtttgaaggccatgaattcccagcacaacggacatgcctatgttcacaacataaactcttaaatgtgtccaaaactagaagaagaaaaaaaaaaaaaaaaaggagcgggagattgggggagagccggagaccctcagtctctcctggaataattcatcattgccctggtcgctcccctccctgggtcccagtcagaagggccctgcgcaggatgcatctggggctctaaggggaggcagccccagcccccccagagcctgacccctgctttctccctccttcccaccgagcccagcctgcggggagagcctgaaggcccctcgggtggtgggcggggagaaggcctctgtggattcctggccctggcaggtcagcatccagtacaacaaacagcacatctgtggagggagcatcctggacccccactggatcctcacggcagcccactgcttctggtaaggcccaccggggaggaggtctcgggggtcaagtccgtggaagggcctgagttgttttctgatgccctctcccctcccccaccccaaagaccaggcaccagggtgtggccaccccagtgggccagtggatgtgatgtcctttgttaggccttgggggtctaagcagagggtgctggtcaccaagcctgacgtctccccttcgaggtaggaagccatcggtacccagtgtcctcagtccacctccatgtccttcactaaacaagagctcttcccatcgcctggcatcctgagaaccctctccctctgtgccagtcttctacgaagtggttctggtccagggtccctgccaccctcccattatacccagaaacaataacaacaaccaaacatagagactctcttacaggccagcagagaaaagcctgcccaaacctctggggaaggaccactccagggcctcttcgttttagtcactcattcagcaattatgagcacctgctaggtaccaagcactgtattacctgcccacctgcattaaactccatctctggcttagaggagcgcacagtgtagacaagggatttgtaactgtttttgagactttgacaaaagctataaatatttccctggaaaaccatccctgcacacccagctgtgtccaatgctgggaggaagcagtatggaatggaaccaaccacaacgaaagcaccacgggaggaacatggccaagaaaaagccttggaggagagagtgtcaggacccggagctcaggccttgggaacagccccttccccaggccaccagttagaagtttccttgggcaggtttcaccagggaaaaggatcagattgccctttagaatctgacctgaggtcgggcattcctcagactgcccttctccaccagccccacccccaccatcctcacctccaaaggcagggccatctggcaccccaagccccagggccctgcgcacccagcctcctcatcttgcctcagacctgaaccccactgtctctgcccccaggaagcatctcgatgtgcccaactggaaggtgagggccggctcagacaaactgggcaacttcccatccctgcctgtggccaagatcttcatcactgagcccaacagcacgtcccccaaagagaaagacattgcccttgtgaagctgcagctcccgctcacgttctccggtgagagacggcctccctgccgtcagggagggctcaggccctgggcatatccacttttcaagtcctcagtttgccccctgtttccagcaaatatgtcagcctcacatcactgaaggcaggaagcccctcaagctgtcagctgaccccccatcctgaggtctgttttccccacgtggagcccctcgggtgagaatcacagatcacccattcctggagtttgtgagggacctgaaagatggtctgacccatcctccacccgaccaatggcaagagcctgctcagtctccctgcccaggggtcctggggcccctttgccctcctcagggggcagggaaatcttccgttagggcagcccgcagaatctctgtacagctctgctcagtaactcatgcttatcttgggttgtacttggatcagcatgtctcatggtgggttgcataggacatgggttccttggagaaaagagaaagtttgggaaagatttttatactacagctcttttcttggagcgcattagtatatcaatgatatgagaggtcttttaaaaattttgaacagttcgttgtgttcaagtcatgatttgataaacttatctgagcacagacactttttcttgtgccgcttctattaacaacccctggaacgtgtgctatagagagcttagctgccaggctctcctcaacctcgggtcccctcttcctctctgctcctgataggcacagtcaggcccatctgcctgcccttctctgacgaggaactcactccaggcaccccagtctgggtcatcggctggggctttacggaacctaatggaggtaagtcccaggctcaggaccacagcactgagtgagcagctcccagaggtgatggcaaggaggaccgtccttcagaaaaacccatcctggaggtgcaagcacccaggtgccaggcaacccggagaattttcctgggctgggggcagagggcaagaatgggagtgtgagtgtgttttcaccctccagggaagatgtctgatcatctgctgcaggcgtccgtccaggtcattaaccgcactcggtgcaacgcggaggatgcgtaccagggggaagtcacggagaagatgctgtgtgcaggcctcctggagggcggtgtggacacctgccaggtagggcctccaaggatcgtgagggaattacagagagagggctgtctgccctgaagagatgagagagttcaggggccaaatgccccacaggaagtcttcttgcatcacactccagtgtgtgatggagggaggaagggagaggaaaggaaggaaggacactagtcagataaagatctaagaatggatggatgatggatgcctggatggatagatagatagacagacagacagacagacagacagatagatgcaggaacatttcacacataaacttgatggttcccccccacacacacaccttgctcttacttccatcatctgcagcagtgtgagcccacaaaggggaggaatcccaggtctgttttctccataacagtcctaagctgctgaactaaactagagaaaacccatctcccacaggccgatccctggtgcactacccctcacgccatccccagtggctaaacattgaaaatattctcacacacacaccccccccaggatctcctgaaccatttccaagaaaaccacaaaccctactcccacccccacccccatctgccgtcatcaaattcctttacctaggaggcatctgctgatgacttgatctctgatgtcaggtaaatgctggatttgggggacagtatcttccctcagggagctcacggtagtgagggtggggttgcaaacaaaacaaaacaaaacaaacaggcagttccgctatagtgttattaggttctgagggcgggagccccaggggctgtaattgttggtaagaaggacacgcaaacacgcacgctcttcactgtctgacgtctttttttttttaatttggttgtttacttgtctcctctcccccaccgtactttctctctcactcactctccctctctgtctctccccccactcctgccccatttgattctaagctttttttttttttttaattgattcccgcttgtctagaacagaggctggcatgatacaggccctcctgaaatgggacttacggtacatttgggacctattaggttaagcaaagacaagccggcttccttagtgcaggaccctgtacagccctcaccgctagcgtgagctgtggatttctggcagagggctaactgcatgcccacaacctacttaaccgcaggcagaaccctttggacccagaagggaaggatgttccaggcggcagggaagagcataagcagaggaatgagggtagagcagcagagagtgacggagagcaaggaacggatgggcctgtgcagggaggggagggaagaaaagcacccccctccaccacacttctgcccaccctccccagcagcccctgctctggcctcccacagggtgacagtggtgggcctctgatgtatcactctggccggtggcaagcagtaggcatcgtgagctggggctatggctgtggggggccgagtacccctggagtatacaccaaggtcacagcctacctcgactggatctacaatgttcggaaggtgagcctgtttgccctccctgctgtgccctccctctgttctgctcccctgagggtaccaaagcatccttaggttttatttaactggctctgagaaacccctttccatcccaaataatcttccctcccagcaagtgataacctgggattccaccattaagagctgaaattccttaaatcagagattcacgttaaatgcaaataccttctggcatggaacacccccagctttaccacgctctcttctctggcactttcctggagtttccccatggctccgcaggttaaggatctggcattgtcactgctgtggctcaggtcactactggcatacccattcgatccgcggcccaggaatttccacatgctgcaggcagggccggaaaaaaaggacacgttccagagaacatattctgataagaaggaagatagaaaggcttacgaggttttctggcagcaaagagacagcaaagagcttcagaaaaggaagattaggagttcccgtcgtggcgcagtggttaacgaatccgactaggaaccatgaggttgcgggttcggtccctgcccttgctcagtgggttaacgatccagcgttgccgtgagctgtggtgtaggtcgcagacgcggctcggatcccgcgttgctgtggctctggcgtaggccggtggctacagctctgattcaacccctagcctgggaacctccatacgccgcgggagcggcccaagaaatagcaacaacagcaacaacaacaacaacagacaaaaagacaaaagacaaaaaaaaaaaaaagaaaaggaagattaattttacacgccaccattttgtcccatttcaagtcatctgatttctttgttggtctctttgattctagtctgatccatgatgctgctgcccacctgcagtgctggaaactgcttccctcctgccctgcccacctggggatccaccagaagtcaggcacagagcaagagcccccttgggcccatctctctgcaatctcggcattccttggcacagcaaaggacctccactcctgtaagagaccccacagcccacaggcacccaggagaaagtcagcagccccagctcagataccctgacgcttccagcatcccaaggaaggagagacacagcccacaagccagcctccaggccaaggtcttggaagacattgctaggccaaaaaggaaatttctactgaagaaaagaatggacatagatgctcttctcagagcccagatcaccacgagctagagtggagggaggaaagggtgtgtgccagccctctccttccctacccatccccaagcccacccaaacgaggaagcatttagaatgtaaaatgcactgtcctgctgtttgcactatcatttcttactattgttattactgctacctctatggccactgtttattatactggtgggtgagctccttgaggctgcatggtgagagctgagctcaaactactgacgcatgatctgtactttggcctgggtaggaaaaggtcttggggagatgattgacactcaaagcagaagaagaggaaaaatggagaccagatctgatgggtagaagcaacagtgaccagccaactggtaaagccagcatcagtcagtaagtctcctttgcacacacgtctcatcccactgtccttctctccttcctccctgccgtgatataccctcaacttgggaggaagagaatttggaccttcaaggctgacattacatcctggctccccaagtggctagaaggaggtattgcacctctaagtcacaacctggaaagccctggctgtggtttttatctttcgctttttaaaaaagtcatttatggggttccttagtagcccagtggttaagtattcagcattgtcactgctgtggcttgggttcaatccctggcctggaaacttccacatgcagccaaaattaaataaataaaaaccaaaaataaaaacaaataaacaggagttcccgtcgtggcgcagtggttaacgaatccgactaggaaccatgaggttgcaggttcgatccctgccgttgctcagtgggttaacgatccggtgttgcggtgagctgtggtgtaggtcacagacacggctcgaatcccgagttgctgtggttctggcataggctggcagctacggctccgattagacccctaggctgggaacctccatgtgccgcgggagtggcccaagaaatggcaaaaaaaaataaaaataaaaataaaataaacaaaatatttatcat

SEQ ID NO: 442 - protein, 435 aa, Sus scrofa
MDPASDQPLNSLDVAPQRKPRTPLQTFKKVGIPIIAALLGLAAIVVVAILIKVILDKYYFLCWQPLHFIPREQLCDGQQDCASGEDEQLCVKKFPDGPPVAVRLSRDRSTLQVLHPATQTWASACFDNFTEALAKTACGQMGYDSKPTFRAVRIGPDQDLDVVKITENDQELRVQNSSGPCLSGSLVSLHCLACGESLKAPRVVGGEKASVDSWPWQVSIQYNKQHICGGSILDPHWILTAAHCFWKHLDVPNWKVRAGSDKLGNFPSLPVAKIFITEPNSTSPKEKDIALVKLQLPLTFSGTVRPICLPFSDEELTPGTPVWVIGWGFTEPNGGKMSDHLLQASVQVINRTRCNAEDAYQGEVTEKMLCAGLLEGGVDTCQGDSGGPLMYHSGRWQAVGIVSWGYGCGGPSTPGVYTKVTAYLDWIYNVRKELW

SEQ ID NO: 443 - protein, 397 aa, Sus scrofa
MDPASDQPLNSLVKVILDKYYFLCWQPLHFIPREQLCDGQQDCASGEDEQLCVKKFPDGPPVAVRLSRDRSTLQVLHPATQTWASACFDNFTEALAKTACGQMGYDSKPTFRAVRIGPDQDLDVVKITENDQELRVQNSSGPCLSGSLVSLHCLACGESLKAPRVVGGEKASVDSWPWQVSIQYNKQHICGGSILDPHWILTAAHCFWKHLDVPNWKVRAGSDKLGNFPSLPVAKIFITEPNSTSPKEKDIALVKLQLPLTFSGTVRPICLPFSDEELTPGTPVWVIGWGFTEPNGGKMSDHLLQASVQVINRTRCNAEDAYQGEVTEKMLCAGLLEGGVDTCQGDSGGPLMYHSGRWQAVGIVSWGYGCGGPSTPGVYTKVTAYLDWIYNVRKELW

SEQ ID NO: 444 - protein, 435 aa, Sus scrofa
MDPASDQPLNSLDVAPQRKPRTPLQTFKKVGIPIIAALLGLAAIVVVAILIKVILDKYYFLCWQPLHFIPREQLCDGQQDCASGEDEQLCVKKFPDGPPVAVRLSRDRSTLQVLHPATQTWASACFDNFTEALAKTACGQMGYDSKPTFRAVRIGPDQDLDVVKITENDQELRVQNSSGPCLSGSLVSLHCLACGESLKAPRVVGGEKASVDSWPWQVSIQYNKQHICGGSILDPHWILTAAHCFWKHLDVPNWKVRAGSDKLGNFPSLPVAKIFITEPNSTSPKEKDIALVKLQLPLTFSGTVRPICLPFSDEELTPGTPVWVIGWGFTEPNGGKMSDHLLQASVQVINRTRCNAEDAYQGEVTEKMLCAGLLEGGVDTCQGDSGGPLMYHSGRWQAVGIVSWGYGCGGPSTPGVYTKVTAYLDWIYNVRKSDP

SEQ ID NO: 445 - DNA, 200 bp, Sus scrofa
  source 1..200: mol_type = other DNA
aagagcgaaggcagacccggaactcgggttagagagagaaacacaaaaaggtcctacttgaagagcacatcgtcctggatcggggtttgggctgcctgctacgccccatagggcctgtgggccccggggggctgcggggggtagacactttcaggttggtatccgtggttttcatagtaaggcccgacacctggccgcga

SEQ ID NO: 446 - DNA, 200 bp, Sus scrofa
  source 1..200: mol_type = other DNA
ctgcgcacccagcctcctcatcttgcctcagacctgaaccccactgtctctgcccccaggaagcatctcgatgtgcccaactggaaggtgagggccggctaagagaaagacattgcccttgtgaagctgcagctcccgctcacgttctccggtgagagacggcctccctgccgtcagggagggctcaggccctgggcata